(19) United States
(12) Patent Application Publication
(10) Pub. No.: US 2004/0253634 A1
(43) Pub. Date: Dec. 16, 2004

(54) Novel microarrays and methods of use thereof

(76) Inventor: Denong Wang, New York, NY (US)

Correspondence Address: John P White, Cooper & Dunham, 1185 Avenue of the Americas, New York, NY, 10036, US

(21) Appl. No.: 10/474,449

(22) PCT Filed: Apr. 10, 2002
(86) PCT No.: PCT/US02/11612

Related U.S. Application Data
(60) Provisional application No. 60/282,926, filed on Apr. 10, 2001.

(51) Int. Cl. G01N 33/53; C12M 1/34; G01N 33/543

(52) U.S. Cl. 435/7.1; 435/287.2; 436/518

(57) ABSTRACT

This invention provides novel nitrocellulose-based or llydrogel-based microarrays and methods of making and using them (1) to detect the presence of one or more agents in a sample, (2) to determine the amount of one or more agents in a sample, (3) to determine whether a subject is afflicted with a disorder, and (4) to determine whether an agent known to specifically bind to a first compound also specifically binds to a second compound. This invention also provides kits which comprise the instant micoarrays. This invention further provides antibodies capable of specifically binding to a glycomer present both on the surface of a mammalian macrophage or intestinal epithelial cell, and on a bacterial cell. Finally, this invention provides diagnostic methods using the instant antibodies.

This invention is a continuation-in-part and claims the benefit of U.S. Provisional Application No. 60/282,926, filed Apr. 10, 2001, the contents of which are hereby incorporated by reference into this application.

Throughout this application, various references are cited. Disclosure of these references in their entirety is hereby incorporated by reference into this application to more fully describe the state of the art to which this invention pertains.

The invention described herein was made with Government support under grant number AI45326 from the National Institutes of Health. Accordingly, the United States Government has certain rights in this invention.

### BACKGROUND OF THE INVENTION

Genomics

The Human Genome Project is rapidly approaching its end: the complete mapping and sequencing of the human genome, and the identification of all genes therein. Emerging from this effort is a new generation of biotechnologies, collectively known as “functional genomics”. These technologies, including DNA chips (1) and cDNA microarrays (2,3), make use of the sequence information and genetic materials provided by the human genome project, combine advanced laser and fluorescence sensor technology, and take advantage of computer-aidedlarge-scale data management systems. Differing from classical molecular biology methods which focus on a specific gene or its product, these new approaches monitor the expression of genes on a genome-wide scale, and identify their characteristic overall patterns. The scope of biological investigation has therefore been expanded from the study of a single gene or protein to the study of numerous genes and/or proteins simultaneously.

Proteomics

Proteins are the final gene products, acting as fundamental elements of living organisms. However, the amount of mRNA expression does not always indicate the level of its encoded protein in a cell. The protein molecule has its own life span and kinetics of metabolism. There are specialized cellular machineries, such as the ubiquitin-dependent and -independent pathways of protein degradation, allowing rapid turnover of a protein when its function is no longer required. The fate of a newly synthesized protein is also significantly influenced by post-translational modifications, such as phosphorylation, glycosylation, acetylation or myristylation, at specific amino acid residues. Such molecular modifications are frequently regulated differentially and/or developmentally, establishing a specific function, or playing a structural role, for a given protein. Thus, it has been generally accepted that a better understanding of the genome's function will not be possible without protein analysis. Developing technologies for a genome-wide analysis of protein expression and post-translational modification represents a major challenge to the scientific community (4,5).

Glycomics Carbohydrate-containing macromolecules are the secondary products of genes. Their synthesis requires multiple enzymatic reactions and many steps of intracellular trafficking, transportation and modification. Multiple genes contribute to the synthesis of cellular elements containing complex carbohydrates. “Glycomics”, a new scientific discipline, has emerged to create a comprehensive understanding of the structure, function, synthesis and genetic regulation of cellular carbohydrate molecules.

Carbohydrates are abundant on cell surfaces, existing as either membrane-bound glycoconjugates or secreted substances. These molecules play fundamental structural and protective roles. They are also abundant intracellularly, and serve as an active and dynamic energy reservoir.

Recent studies further demonstrated that many important signaling and regulatory processes are mediated by the interaction of carbohydrate-ligands and their receptors (6,7). Abnormal expression of carbohydrate moieties may occur in cells that are undergoing malignant transformation. These moieties may therefore serve as molecular targets for tumor diagnosis or therapy.

The carbohydrate molecules of microorganisms are important in establishing the biological relationships of microbes and their hosts (7-9). These relationships especially include the host recognition of microorganisms and the induction of an immune response by a microbial antigen. The carbohydrate moieties of microbial antigens frequently serve as the key structures for immune recognition (10). Identifying such determinants is of fundamental importance for understanding the molecular mechanisms of host recognition and immune responses.

Existing Technologies

Technologies suitable for monitoring protein expression on a genome-wide scale and for characterizing a wide range of ligand-receptor interactions such as protein-protein protein reactions, carbohydrate-protein reactions and the interaction of synthetic small molecules and cellular components have yet to be developed. Current methods for specifically detecting and quantifying a protein or a microbial polysaccharide include antigen/antibody based-immunoassays. These assays include (a) classical direct immunoassays, such as immunodiffusion, immunoelectrophoresis, agglutination and immunoprecipitation assays, and (b) recently developed methods such as immunofluorescence, radioimmunoassay (RIA), enzyme-immunoassay (EIA) and western blot assays. These approaches exploit the specificity of antigen-antibody interactions. However, they are designed for analyzing only one agent at a time, and are therefore limited as to the number of molecules that can be analyzed in a single assay.

In sum, a single technology useful for the simultaneous study of numerous molecules, be they protein, carbohydrate or combinations thereof, is sorely needed to advance both proteomics and glycomics.

### SUMMARY OF THE INVENTION

This invention provides four microarrays and two articles useful for making same. The first microarray comprises a nitrocellulose or Hydrogel support having affixed to its surface at discrete loci a plurality of compounds, wherein (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

The second microarray comprises a plurality of nitrocellulose or Hydrogel supports, each support having one or a plurality of compounds affixed to its surface at a single discrete locus or a plurality of compounds affixed to its surface at discrete loci, wherein (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

The first article comprises a nitrocellulose or Hydrogel support having dextran affixed to its surface at discrete loci. In one embodiment the dextran is α(1,6)dextran.

The third microarray comprises the first article, wherein at least one compound is affixed to the dextran at each discrete locus, the composition of compounds at each discrete locus differing from the composition of compounds at at least one other discrete locus.

The second article comprises a plurality of nitrocellulose or Hydrogel supports, each support having dextran affixed to its surface at one or more discrete loci. In one embodiment the dextran is α(1,6)dextran.

The fourth microarray comprises the second article, wherein at least one compound is affixed to the dextran at each discrete locus, the composition of compounds at each discrete locus differing from the composition of compounds at at least one other discrete locus.

This invention provides three methods for detecting the presence of agents in a sample. The first method is a method of detecting in a sample the presence of one or more agents which specifically bind to one or more known glycomers, which method comprises: (a) contacting the sample with the first or second microarray, wherein each known glycomer is affixed at at least one discrete locus and wherein the contacting is performed under conditions which would permit an agent, if present in the sample, to specifically bind to its corresponding glycomer in the microarray; and (b) determining whether any known glycomer in the microarray has an agent specifically bound thereto, thereby detecting the presence of the one or more agents in the sample.

The second method is a method of detecting in a sample the presence of one or more agents which specifically bind to one or more known insoluble proteins, which method comprises: (a) contacting the sample with the first or second microarray, wherein each known insoluble protein is affixed at at least one discrete locus and wherein the contacting is performed under conditions which would permit an agent, if present in the sample, to specifically bind to its corresponding insoluble protein in the microarray; and (b) determining whether any known insoluble protein in the microarray has an agent specifically bound thereto, thereby detecting the presence of the one or more agents in the sample.

The third method is a method of detecting in a sample the presence of one or more agents which specifically bind to one or more known antibodies or lectins, which method comprises: (a) contacting the sample with the first or second microarray, wherein each known antibody or lectin is affixed at at least one discrete locus and wherein the contacting is performed under conditions which would permit an agent, if present in the sample, to specifically bind to its corresponding antibody or lectin in the microarray; and (b) determining whether any known antibody or lectin in the microarray has an agent specifically bound thereto, thereby detecting the presence of the one or more agents in the sample.

This invention further provides three quantitative methods. The first method is a method of determining the amount of one or more agents in a sample, each of which specifically binds to one or more known glycomers, which method comprises: (a) contacting the sample with the first or second microarray, wherein each known glycomer is affixed at at least one discrete locus, and wherein the contacting is performed under conditions which would permit an agent, if present in the sample, to specifically bind to its corresponding glycomer in the microarray; (b) for each known glycomer in the microarray, determining the amount of agent specifically bound thereto; and (c) comparing the amounts so determined to a known standard, thereby determining the amount of the one or more agents in the sample.

The second method is a method of determining the amount of one or more agents in a sample, each of which specifically binds to one or more known insoluble proteins, which method comprises: (a) contacting the sample with the first or second microarray, wherein each known insoluble protein is affixed at at least one discrete locus, and wherein the contacting is performed under conditions which would permit an agent, if present in the sample, to specifically bind to its corresponding insoluble protein in the microarray; (b) for each known insoluble protein in the microarray, determining the amount of agent specifically bound thereto; and (c) comparing the amounts so determined to a known standard, thereby determining the amount of the one or more agents in the sample.

The third method is a method of determining the amount of one or more agents in a sample, each of which specifically binds to one or more known antibodies or lectins, which method comprises: (a) contacting the sample with the first or second microarray, wherein each known antibody or lectin is affixed at at least one discrete locus, and wherein the contacting is performed under conditions which would permit an agent, if present in the sample, to specifically bind to its corresponding antibody or lectin in the microarray; (b) for each known antibody or lectin in the microarray, determining the amount of agent specifically bound thereto; and (c) comparing the amounts so determined to a known standard, thereby determining the amount of the one or more agents in the sample.

This invention further provides three diagnostic methods. The first method is a method of determining whether a subject is afflicted with a disorder characterized by the presence or absence in an afflicted subject of an agent which specifically binds to a known glycomer, which method comprises: (a) contacting a suitable sample from the subject with the first or second microarray, wherein the known glycomer is affixed at at least one discrete locus and wherein the contacting is performed under conditions which would permit the agent, if present in the sample, to specifically bind to the known glycomer in the microarray; and (b) determining whether the known glycomer in the microarray has the agent specifically bound thereto, thereby determining whether the subject is afflicted with the disorder.

The second method is a method of determining whether a subject is afflicted with a disorder characterized by the presence or absence in an afflicted subject of an agent which specifically binds to a known insoluble protein, which method comprises: (a) contacting a suitable sample from the subject with the first or second microarray, wherein the known insoluble protein is affixed at at least one discrete locus and wherein the contacting is performed under conditions which would permit the agent, if present in the sample, to specifically bind to the known insoluble protein in the microarray; and (b) determining whether the known insoluble protein in the microarray has the agent specifically bound thereto, thereby determining whether the subject is afflicted with the disorder.

The third method is a method of determining whether a subject is afflicted with a disorder characterized by the presence or absence in an afflicted subject of an agent which specifically binds to a known antibody or lectin, which method comprises: (a) contacting a suitable sample from the subject with the first or second microarray, wherein the known antibody or lectin is affixed at at least one discrete locus and wherein the contacting is performed under conditions which would permit the agent, if present in the sample, to specifically bind to the known antibody or lectin in the microarray; and (b) determining whether the known antibody or lectin in the microarray has the agent specifically bound thereto, thereby determining whether the subject is afflicted with the disorder.

This invention further provides a method of determining whether an antibody known to specifically bind to a first glycomer also specifically binds to a second glycomer, which method comprises: (a) contacting the antibody with the first or second microarray, wherein a plurality of glycomers, other than the first glycomer, are affixed at discrete loci in the microarray, and wherein the contacting is performed under conditions which would permit the antibody to specifically bind to the first glycomer if it were present in the microarray; and (b) determining whether any of the glycomers in the microarray, other than the first glycomer, has the antibody specifically bound thereto, thereby determining whether the antibody also specifically binds to a second glycomer.

This invention further provides a method of determining whether an antibody known to specifically bind to a first insoluble protein also specifically binds to a second insoluble protein, which method comprises: (a) contacting the antibody with the first or second microarray, wherein a plurality of insoluble proteins, other than the first insoluble protein, are affixed at discrete loci in the microarray, and wherein the contacting is performed under conditions which would permit the antibody to specifically bind to the first insoluble protein if it were present in the microarray; and (b) determining whether any of the insoluble proteins in the microarray, other than the first insoluble protein, has the antibody specifically bound thereto, thereby determining whether the antibody also specifically binds to a second insoluble protein.

This invention further provides a method of making a microarray comprising a nitrocellulose or Hydrogel support having affixed to its surface at discrete loci a plurality of compounds, which method comprises contacting the nitrocellulose or Hydrogel support with the compounds under suitable conditions, whereby (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

This invention further provides a method of making a microarray comprising a plurality of nitrocellulose or Hydrogel supports, each support having one or a plurality of compounds affixed to its surface at a single discrete locus or a plurality of compounds affixed to its surface at discrete loci, which method comprises contacting the nitrocellulose or Hydrogel supports with the compounds under suitable conditions, whereby (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

This invention further provides a method of making the first article comprising contacting a nitrocellulose or Hydrogel support with dextran at discrete loci under suitable conditions.

This invention further provides a method of making the second article comprising contacting a plurality of nitrocellulose or Hydrogel supports with dextran, whereby each support has dextran affixed to its surface at one or more discrete loci.

This invention further provides six kits. The first kit comprises one of the instant microarrays and instructions for use. The second kit comprises one of the instant microarrays and a desiccant. The third kit comprises one of the instant microarrays immersed in an aqueous solution.

The fourth kit is a kit for practicing the first diagnostic method, which comprises: (a) a microarray comprising a nitrocellulose or Hydrogel support having affixed to its surface at discrete loci a plurality of compounds, wherein (i) at at least one discrete locus is affixed the glycomer to which the agent present or absent in an afflicted subject specifically binds, and (ii) the composition of compounds at each discrete locus differs from the composition of compounds at at least one other discrete locus; and (b) instructions for use.

The fifth kit is a kit for practicing the second diagnostic method, which comprises: (a) a microarray comprising a nitrocellulose or Hydrogel support having affixed to its surface at discrete loci a plurality of compounds, wherein (i) at at least one discrete locus is affixed the insoluble protein to which the agent present or absent in an afflicted subject specifically binds, and (ii) the composition of compounds at each discrete locus differs from the composition of compounds at at least one other discrete locus; and (b) instructions for use.

The sixth kit is a kit for practicing the third diagnostic method, which comprises: (a) a microarray comprising a nitrocellulose or Hydrogel support having affixed to its surface at discrete loci a plurality of compounds, wherein (i) at at least one discrete locus is affixed the antibody or lectin to which the agent present or absent in an afflicted subject specifically binds, and (ii) the composition of compounds at each discrete locus differs from the composition of compounds at at least one other discrete locus; and (b) instructions for use.

This invention further provides a first antibody capable of specifically binding to a glycomer present on the surface of a mammalian macrophage, which glycomer, or structural mimic thereof, is also endogenous to, and present on the surface of, a bacterial cell.

This invention further provides a second antibody capable of specifically binding to a glycomer present on the surface of a mammalian intestinal epithelial cell, which glycomer, or structural mimic thereof, is also endogenous to, and present on the surface of, a bacterial cell.

This invention further provides a method of determining whether a subject is afflicted with a disorder characterized by the presence of a glycomer on the surface of macrophages in an afflicted subject, which glycomer, or structural mimic thereof, is also endogenous to, and present on the surface of, a bacterial cell, comprising: (a) contacting a sample of the subject's macrophages with the first antibody; and (b) determining whether the antibody specifically binds to the macrophages in the sample, such binding indicatingthat the subject is afflicted with the disorder.

Finally, this invention provides a method of determining whether a subject is afflicted with a disorder characterized by the presence of a glycomer on the surface of intestinal epithelial cells in an afflicted subject, which glycomer, or structural mimic thereof, is also endogenous to, and present on the surface of, a bacterial cell, comprising: (a) contacting a sample of the subject's intestinal epithelial cells with the second antibody; and (b) determining whether the antibody specifically binds to the intestinal epithelial cells in the sample, such binding indicating that the subject is afflicted with the disorder.

### BRIEF DESCRIPTION OF THE FIGURES

FIG. 1

This Figure shows a carbohydrate microarray and its application in characterizing the epitope-binding specificity of monoclonal antibodies (“mAb”). Dextran preparations of defined structural characteristics, including N279, LD7, B1299S and B1355S, were immobilized on a nitrocellulose-coated micro-glass slide in serial dilutions and stained with anti-α(1,6)dextran antibodies. These antibodies were either a groove-type antibody, i.e., 4.3. F1, or a cavity-type antibody, i.e., 16.4.12E, and were conjugated with fluorescence. Their distinct epitope-binding specificities were visualized by scanning the carbohydrate microarray using a GMS 418 microarray scanner.

FIG. 2

This Figure shows an antigen-based microarray and its application in studying the cross-reactivity of monoclonal antibodies. Forty-nine distinct antigen preparations, including microbial polysaccharides, blood group substances and other glycoconjugates, were arrayed on slides and incubated with fluorescence-labeled mAbs. FIG. 2A: anti-DEX 4.3. F1; FIG. 2B: anti-DEX 16.4.12E. Intensity values of cross-reacting spots were compared with those of specific binding to α(1,6)dextran N279. N279 was applied as a series of 1:5 dilutions of a 100 μg/ml solution (a). Other antigens were applied as 500 μg/ml. Identical antigens are arrayed in FIG. 2A and FIG. 2B.

FIG. 3

This Figure shows the detection of a cell population in the small intestine of an adult mouse by a groove-type anti-α(1,6)dextran antibody 4.3. F1 (IgG3), which showed cross-reactivity to a chondroitin sulfate B preparation.

The cryostat sections of small intestine were stained either by mAb 4.3. F1 (IgG3) or by an IgG3 isotype control mAb obtained from BD PharMingen. The two mAbs were fluorescent conjugates. Sections were co-stained with DAPI for the cell nucleus to visualize the overall tissue structure. The 4.3. F1positive cells were seen in the lamina propria of the small intestine. FIGS. 3A-3D: mAb 4.3. F1; FIGS. 3E-3H: isotype control.

FIG. 4

This Figure shows that groove-type and cavity-type anti-α(1,6)dextran monoclonal antibodies recognize distinct cellular markers: a groove-type mAb 45.21.1 (IgA) identifies a cell population in the lamina propria of the small intestine (FIGS. 4C and 4F), and a cavity-type mAb 16.4.12E (IgA) stains the epithelial cells in the crypts of the small intestine (FIGS. 4B and 4E). An IgA isotype control mAb purchased from BD PharMingen was applied as a background control (FIGS. 4A and 4D).

FIG. 5

This Figure shows the recognition of a cell population in the human small intestinal tissue using anti-α(1,6)dextran antibodies. The intestinal section of a normal (FIGS. 5A and 5B) and of a celiac individual (FIGS. 5C and 5D) were stained with the fluorescence-conjugate of mAb 16.4.12E (FIGS. 5B and 5D) and co-stained with DAPI to reveal the intestinal structures (FIGS. 5A and 5C).

FIG. 6

This Figure shows the immobilization of polysaccharides on a nitrocellulose-coated glass slide. Panel A: Image of the carbohydrate microarray (microarrays of dextrans and inulin) spots before and after washing. Panel B: Quantitative illustration of the relation of fluorescence intensity and the concentration of printed carbohydrate microarrays before and after washing. Fluorescent conjugates of dextrans or inulin were dissolved in saline (0.9% NaCl) and spotted at an initial concentration of 10 mg/ml and then diluted in serial dilutions of 1:5. The microarray slides were scanned before and after washing. The data of six repeats of the same experiment is statistically analyzed and presented. Legend:

[symbol]

2000k;

[symbol]

70k;

[symbol]

20k;

[symbol]

Inulin.

FIG. 7

This Figure shows the immunological characterization of surface-immobilized dextran molecules. Panel A: Microarray binding curves of a groove-type anti-Dex 4.3F1 (IgG3/Kappa) and a cavity-type anti-dextran 16.4.12E (IgA/Kappa) to dextran molecules of distinct structure. Dextran molecules were printed with an initial concentration of 0.1 mg/ml and diluted by a 1:5 series titration. The printed arrays were washed to remove unbound antigens and then stained with biotinylated anti-dextran, either 4.3F1 or 16.4.12E, at a concentration of 1 μg/ml, and then stained with Cy3-streptavidin at 1:500 dilutions. The readout of the experiment (i.e., fluorescent intensity of the microspot) reflects the amounts of antigen immobilized and epitopes displayed for antibody recognition. The cavity-type mAb 16.4.12E bound to N279 and B12995, but not LD7. By contrast, the groove-type mAb 4.3F1 bound to the dextran preparations N279 and LD7, but bound poorly to B1299S. Panel B: ELISA binding curve of anti-Dex 4.3F1 and 16.4.12E. Dextran preparations were coated on an ELISA plate at an initial concentration of 10 μg/ml and then diluted by a 1:5 series titration in 0.02 M borate-buffered saline, pH 8.0. The antigen-coated plates were incubated with biotinylated anti-dextrans at a concentration of 1 μg/ml. The bound antibodies were revealed with an alkaline phosphatase (AP)-streptavidin conjugate and AP substrate.

Legend:

[symbol]

N279;

[symbol]

LD7;

[symbol]

B-1299S.

Left Panel: 4.3F1 (Groove-type); Right Panel: 16.4.12 (Cavity-Type).

FIG. 8

Bacilluus anthracis exposes and releases a number of antigens of distinct structural characteristics to trigger and induce a comprehensive picture of a host response. Left: Schematic of the life cycle of Bacillus anthracis. Dormant spores present in vitro are highly resistant to adverse environmental conditions. In a suitable environment, spores establish vegetative growth. In an early infection, these infective particles are ingested by the phagocytic cells and accumulate in the local lymphoid tissue. Some may survive from the phagocytes and initiate their germination and vegetative growth. The vegetative form of the bacteria is square-ended and capsulated. In the late infection, they multiply rapidly, express their virulence factors to kill the host and develop to the stage of resporulation in vivo. Antigens & toxins: Vegetative bacillus releases multiple factors, such as toxins, protein factors, and soluble polysaccharides (1-3,4 of the Third Series of Experiments). The protein fractions, such as the protective antigen, named PA, can provide strong protection to the immunized animals. It is now well understood that PA is an integrated component of the lethal toxin of Bacilluus anthracis. It binds to a specific cellular receptor and forms toxic, cell bound complexes with edema factor (EF) and lethal factor (LF) (1-3 of the Third Series of Experiments). Neutralization antibodies to PA or a polyvalent factor that inhibits the formation of the complex may protect animals from the lethal attack by the toxin (5 of the Third Series of Experiments). A considerable amount of polysaccharides are also present in the culture media of the growing bacteria. Its sugar compositions are similar (if not identical) to the cell wall Gal-NAG polysaccharide. Right: An outline of the human immune system. The native immunity forms the first line of a host anti-infection response. These include macrophages, natural killer cells (NK), pre-existing “natural antibody” of IgM isotype and perhaps a specific B cell lineage, the B-1 cells, TCR γδ T cells, and other cells. In the anthrax infection, the phagocytic cells may play multiple roles in the host-microbe interaction. These may include both protective and pathogenic effects (see below for details). The acquired immune system includes B cells (the bone marrow derived B cells, or B-2 cells) and T cells (the thymus derived TCR αβ T cells). B cells mount a specific antibody response to a microbial antigen, either a T-independent antigen, such as an anthrax polysaccharide, or a T-dependent antigen, for example the protective antigen (PA) of B. anthracis; specific T cells can be activated by a TD protein antigen to regulate a B cell responses, either positively (T helper, Th1 and Th2) or negatively. There is also activation of specific cytotoxic T cells (Tc), which can kill the cells that express a foreign antigen. Many host cells, including immune cells and non-immune cell types, may produce cytokines or other inflammation factors to assist a host anti-infection response.

FIG. 9

This Figure shows a simple and efficient procedure for producing a carbohydrate microarray. Microspotting: Carbohydrate antigens were printed using Cartesian Technologies' PIXSYS 5500C (Irvine, Calif.) with STEALTH 3 pins. Supporting substrate: FAST Slides (Industrial partner A, Schleicher & Schuell, Keene, N.H.). The printed carbohydrate microarrays were air dried and stored at room temperature without desiccant before application. Immuno-staining: Immediately before use, the microarrays were rinsed with phosphate-buffered saline (PBS). The staining procedure utilized is essentially identical to regular immunoflourescent staining of tissue sections. Microarray-scanning: A ScanArray 5000 Standard Biochip Scanning System and its QuantArray software (Packard Biochip Technologies, Inc.) were applied for scanning and data capturing.

FIG. 10

This Figure shows a schematic of the 8-chamber sub-arrays.

FIG. 11

This Figure shows probing of the repertoires of human serum antibodies using Antigen Chip 4000. Left: HIV negative normal serum. Riaht: Serum of an HIV-1 infected individual. For each microarry analysis, 10 ml of serum were applied on an antigen chip at 1:10 dilutions. Anti-human antibodies with distinct fluorescent tags were applied to recognize and quantify the bound human IgG, IgM and IgA. In this Figure, human IgG was stained in Red/Cy5 and human IgM in Green/Cy3. The two images of contrasting colors were overlaid. IgA human antibodies were detected on the same chip with an anti-human IgA^{FITC} (data was not shown).

FIG. 12

This Figure shows the scanning of human antibodies specific for a large panel of HIV proteins using a protein-based microarray biochip. Serum specimens of four normal individuals and six AIDS patients were characterized by a protein biochip that displays a large panel of HIV-1 proteins. Each preparation was printed four times on the same biochip. For each assay, 10 μl of serum were applied at 1:10 dilutions on a single chip. Human IgG that was captured by the immobilized antigens was recognized and quantified by a Cy3-labeled second antibody. Data of each group, normal and HIV-infected individuals, were statistically analyzed. Results were presented as the mean value of the ratio of fluorescent intensity over the background of given microspots (Histogram). Their standard division was also shown. Significant variations that were observed in the HIV-1 infected group may reflect the diversity of the HIV-1 specific antibody responses, as well as the level of antigenic cross-reactivities of HIV-1 proteins that were expressed by different clades or strains of HIV-1 virus.

FIG. 13

This Figure shows a schematic of a hypothetical structural and immunological relationships of the type II backbone structure of blood group substances, type XIV pneumococcal polysaccharide and the cell wall Gal-NAG polysaccharide.

FIG. 14

This Figure shows a carbohydrate microarray characterization of human and murine antibodies. Forty-eight distinct antigen preparations were arrayed on slides at antigen concentrations of 0.5 mg/ml and 0.02 mg/ml. They were incubated with combined human serum specimens at a concentration equivalent to 1:100 dilutions of each specimen or with binotinylated mouse monoclonal antibodies at 1 mg/ml. The human IgM captured by microarrays was visualized using an anti-human IgM-AP conjugate and the color developed using Vector Red. The human IgG anti-carbohydrates were detected using a biotinylated anti-human IgG. A Cy3-Streptoavidin conjugate was then applied to visualize the human IgG or murine monoclonal antibodies bound on microarray. The readout of the experiment, i.e., fluorescent intensity of the microspot, reflects the amounts of antigen immobilized and epitopes displayed for antibody recognition. Data of four repeats of microarray staining are summarized in Table 2.

FIG. 15

This Figure shows the prediction of protein structure using TMHMM version 2.0 (55 of the Third Series of Experiments): PX01-54 of B. anthracis encodes a S-layer protein, a novel molecular target for anthrax diagnosis and vaccination.

FIG. 16

This Figure shows biochip detection of human antibody reactivities to either anthrax polysaccharide or Pneumococcus type XIV polysaccharide in mixed human serum specimen which confirms that these antigen preparations are applicable for producing diagnostic microarrays.

### DETAILED DESCRIPTION OF THE INVENTION

### Definitions

As used in this application, except as otherwise expressly provided herein, each of the following terms shall have the meaning set forth below. “Affixed” shall mean attached by any means. In one embodiment, affixed shall mean attached by a covalent bond. In another embodiment, affixed shall mean attached non-covalently.

“Agent” shall mean any chemical entity, including, without limitation, a glycomer, a protein, an antibody, a lectin, a nucleic acid, a small molecule, and any combination thereof.

“Antibody” shall mean (a) an immunoglobulin molecule comprising two heavy chains and two light chains and which recognizes an antigen; (b) polyclonal and monoclonal immunoglobulin molecules; and (c) monovalent and divalent fragments thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human or nonhuman. Nonhuman antibodies may be humanized by recombinant methods to reduce their immunogenicity in man. “Aqueous solution” shall mean any solution in which water is a solvent. Examples of aqueous solutions include water and water-based buffer solutions.

“Complex carbohydrate” shall mean a carbohydrate polymer comprising more than two types of saccharide monomer units. Examples of complex carbohydrates include blood group substances such as Lewis X and Lewis Y.

“Composition of compounds” at a discrete locus shall mean the identity of the one or more compounds at that locus. For example, if locus 1 has compounds A and B, and locus 2 has compounds A and C, then the composition of compounds at locus I differs from that at locus 2.

“Compound” shall mean any molecule. Compounds include, but are not limited to, proteins, nucleic acids, glycomers, lipids and small molecules.

“Dextran” shall mean a branched polymer of glucose consisting mainly of α (1,6)-glycosidic linkages.

“Discrete locus” shall mean a point, region or area for the affixation of a compound which does not overlap with another such point, region or area, and which may further be separated from another such point, region or area by physical space.

“Glycomer” shall mean any carbohydrate-containing moiety. Glycomers include, without limitation, (a) complex carbohydrates, (b) polysaccharides, and (c) glycoconjugates. “Glycoconjugates” include, without limitation, glycoproteins and glycolipids.

“Insoluble protein” shall mean any protein which does not solubilize in aqueous solution. Examples of insoluble proteins include trans-membrane proteins. “Lectin” shall mean a protein that is capable of agglutinating erythrocytes, binding sugars, and/or stimulating mitosis. Examples of lectins include concavalin A.

“Microarray” shall mean (a) a solid support having one or more compounds affixed to its surface at discrete loci, or (b) a plurality of solid supports, each support having one or a plurality of compounds affixed to its surface at discrete loci. The instant microarrays can contain all possible permutations of compounds within the parameters of this invention. For example, the instant microarray can be an all-glycomer microarray, an all-insoluble protein microarray, an all-antibody microarray, a disease-specific microarray, a species-specific microarray, or a tissue-specific microarray.

“Nitrocellulose or Hydrogel support” shall mean any solid support having nitrocellulose or Hydrogel affixed to its surface. Nitrocellulose or Hydrogel supports include, without limitation, nitrocellulose-coated or Hydrogel-coated chips (e.g. silicone chips), slides (e.g. glass slides), filters, plates and beads.

“Polysaccharide” shall mean a carbohydrate polymer comprising either one or two types of saccharide monomer units. Examples of polysaccharides include bacterial cell surface carbohydrates.

“Sample”, when used in connection with the instant methods, includes, but is not limited to, any body tissue, skin lesion, blood, serum, plasma, cerebrospinal fluid, lymphocyte, urine, exudate, or supernatant from a cell culture.

“Specifically bind” shall mean the binding of a first entity to a second entity based on complementarity between the three-dimensional structures of each. In one embodiment, specific binding occurs with a K_{D} of less than 10⁻⁵. In another embodiment, specific binding occurs with a K_{D} of less than 10⁻⁸. In a further embodiment, specific binding occurs with a K_{D} of less than 10⁻¹¹.

“Subject” shall mean any organism including, without limitation, a mouse, a rat, a dog, a guinea pig, a ferret, a rabbit and a primate. In the preferred embodiment, the subject is a human being.

### Embodiments of the Invention

This invention provides four microarrays and two articles useful for making same. The first microarray comprises a nitrocellulose or Hydrogel support having affixed to its surface at discrete loci a plurality of compounds, wherein (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

The second microarray comprises a plurality of nitrocellulose or Hydrogel supports, each support having one or a plurality of compounds affixed to its surface at a single discrete locus or a plurality of compounds affixed to its surface at discrete loci, wherein. (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

The first article comprises a nitrocellulose or Hydrogel support having dextran affixed to its surface at discrete loci. In one embodiment the dextran is α(1,6)dextran.

The third microarray comprises the first article, wherein at least one compound is affixed to the dextran at each discrete locus, the composition of compounds at each discrete locus differing from the composition of compounds at at least one other discrete locus.

The second article comprises a plurality of nitrocellulose or Hydrogel supports, each support having dextran affixed to its surface at one or more discrete loci. In one embodiment the dextran is α(1,6)dextran.

The fourth microarray comprises the second article, wherein at least one compound is affixed to the dextran at each discrete locus, the composition of compounds at each discrete locus differing from the composition of compounds at at least one other discrete locus.

In one embodiment of the first and third microarrays, the nitrocellulose or Hydrogel support is selected from the group consisting of a chip, a slide, a filter, and a plate. In one embodiment of the second and fourth microarrays, the nitrocellulose or Hydrogel support is selected from the group consisting of a chip, a slide, a filter, a plate, and a bead.

In one embodiment of the above microarrays, the number of discrete loci is at least 100. In another embodiment, the number of discrete loci is at least 1000. In a further embodiment, the number of discrete loci is at least 10,000. In a further embodiment, the number of discrete loci is at least 50,000.

In one embodiment of the first and second microarrays, a glycomer is affixed at at least one locus. In another embodiment, an insoluble protein is affixed at at least one locus. In another embodiment, a lectin is affixed at at least one locus. In a further embodiment, an antibody is affixed at at least one locus. In a further embodiment, the microarray has affixed to its surface two or more compounds selected from the group consisting of a glycomer, an insoluble protein, a lectin and an antibody. In a further embodiment, the microarray has further affixed to its surface a compound selected from the group consisting of a soluble protein, a nucleic acid and a small molecule.

In one embodiment of the third and fourth microarrays, a glycomer is affixed to the dextran at at least one locus. In another embodiment, an insoluble protein is affixed to the dextran at at least one locus. In another embodiment, a lectin is affixed to the dextran at at least one locus. In a further embodiment, an antibody is affixed to the dextran at at least one locus. In another embodiment, the microarray has affixed to the dextran two or more compounds selected from the group consisting of a glycomer, an insoluble protein, a lectin and an antibody. In another embodiment, the microarray has affixed to its surface a compound selected from the group consisting of a soluble protein, a nucleic acid and a small molecule.

In one embodiment of the instant microarrays, at each locus is affixed only one compound. In another embodiment, at at least one locus is affixed a plurality of compounds.

This invention provides three methods for detecting the presence of agents in a sample. The first method is a method of detecting in a sample the presence of one or more agents which specifically bind to one or more known glycomers, which method comprises: (a) contacting the sample with the first or second microarray, wherein each known glycomer is affixed at at least one discrete locus and wherein the contacting is performed under conditions which would permit an agent, if present in the sample, to specifically bind to its corresponding glycomer in the microarray; and (b) determining whether any known glycomer in the microarray has an agent specifically bound thereto, thereby detecting the presence of the one or more agents in the sample.

The second method is a method of detecting in a sample the presence of one or more agents which specifically bind to one or more known insoluble proteins, which method comprises: (a) contacting the sample with the first or second microarray, wherein each known insoluble protein is affixed at at least one discrete locus and wherein the contacting is performed under conditions which would permit an agent, if present in the sample, to specifically bind to its corresponding insoluble protein in the microarray; and (b) determining whether any known insoluble protein in the microarray has an agent specifically bound thereto, thereby detecting the presence of the one or more agents in the sample.

The third method is a method of, detecting in a sample the presence of one or more agents which specifically bind to one or more known antibodies or lectins, which method comprises: (a) contacting the sample with the first or second microarray, wherein each known antibody or lectin is affixed at at least one discrete locus and wherein the contacting is performed under conditions which would permit an agent, if present in the sample, to specifically bind to its corresponding antibody or lectin in the microarray; and (b) determining whether any known antibody or lectin in the microarray has an agent specifically bound thereto, thereby detecting the presence of the one or more agents in the sample.

In one embodiment of the above methods, the agent is an antibody which correlates with a disease. In a further embodiment of the first method, the agent is an antibody which correlates with an inflammatory disease. In additional embodiments of the above methods, the agent is an antibody which correlates with an infection or the presence of a tumor.

In one embodiment of the instant methods, the method comprises detecting the presence of a plurality of agents in the sample, each of which binds to either a plurality of glycomers, a plurality of insoluble proteins, or a plurality of lectins or antibodies, as applicable. In another embodiment of the instant methods, the method comprises determining the amount of a plurality of agents in the sample, each of which binds to either one glycomer, one insoluble protein or one lectin or antibody, as applicable.

“Determining” whether an agent is bound to a compound in a microarray can be performed according to methods well known in the art. Such methods include, but are not limited to, fluorescence, radioimmunoassay, and immunolabeling detection.

In the instant methods of detection, several embodiments are provided which include, without limitation, the following: (a) one agent in a sample binds to one compound on the instant microarray; (b) one agent in a sample is detected that binds to more than one compound on the microarray; (c) the collective presence of a plurality of agents in a sample is detected, wherein each such agent binds to one or more compounds on the microarray; and (d) each of a plurality of agents in a sample is individually detected, wherein each such agent binds to one or more compounds on the microarray.

This invention further provides three quantitative methods. The first method is a method of determining the amount of one or more agents in a sample, each of which specifically binds to one or more known glycomers, which method comprises: (a) contacting the sample with the first or second microarray, wherein each known glycomer is affixed at at least one discrete locus, and wherein the contacting is performed under conditions which would permit an agent, if present in the sample, to specifically bind to its corresponding glycomer in the microarray; (b) for each known glycomer in the microarray, determining the amount of agent specifically bound thereto; and (c) comparing the amounts so determined to a known standard, thereby determining the amount of the one or more agents in the sample.

The second method is a method of determining the amount of one or more agents in a sample, each of which specifically binds to one or more known insoluble proteins, which method comprises: (a) contacting the sample with the first or second microarray, wherein each known insoluble protein is affixed at at least one discrete locus, and wherein the contacting is performed under conditions which would permit an agent, if present in the sample, to specifically bind to its corresponding insoluble protein in the microarray; (b) for each known insoluble protein in the microarray, determining the amount of agent specifically bound thereto; and (c) comparing the amounts so determined to a known standard, thereby determining the amount of the one or more agents in the sample.

The third method is a method of determining the amount of one or more agents in a sample, each of which specifically binds to one or more known antibodies or lectins, which method comprises: (a) contacting the sample with the first or second microarray, wherein each known antibody or lectin is affixed at at least one discrete locus, and wherein the contacting is performed under conditions which would permit an agent, if present in the sample, to specifically bind to its corresponding antibody or lectin in the microarray; (b) for each known antibody or lectin in the microarray, determining the amount of agent specifically bound thereto; and (c) comparing the amounts so determined to a known standard, thereby determining the amount of the one or more agents in the sample.

In one embodiment of the instant quantitative methods, the agent is an antibody which correlates with a disease. In a further embodiment of the first method, the agent is an antibody which correlates with an inflammatory disease. In additional embodiments of the above methods, the agent is an antibody which correlates with an infection or the presence of a tumor.

In one embodiment of the instant quantitative methods, the method comprises determining the amount of a plurality of agents in the sample, each of which binds to either a plurality of glycomers, a plurality of insoluble proteins, or a plurality of lectins or antibodies, as applicable. In another embodiment of the instant quantitative methods, the method comprises determining the amount of a plurality of agents in the sample, each of which binds to either one glycomer, one insoluble protein or one lectin or antibody, as applicable.

“Determining” the amount of an agent which is bound to a compound in a microarray can be performed according to well known methods in the art. The “known standards” useful for the instant quantitative methods include, for example, correlations between known concentrations of agents in a control sample and their corresponding values as determined using the instant microarray.

In the instant quantitative methods, several embodiments are provided which include, without limitation, the following: (a) one agent in a sample binds to one compound on the instant microarray; (b) one agent in a sample is quantitated that binds to more than one compound on the microarray; (c) the collective amount of a plurality of agents in a sample are quantitated, wherein each such agent binds to one or more compounds on the microarray; and (d) each of a plurality of agents in a sample is individually quantitated, wherein such agent binds to one or more compounds on the microarray.

This invention further provides three diagnostic methods. The first method is a method of determining whether a subject is afflicted with a disorder characterized by the presence or absence in an afflicted subject of an agent which specifically binds to a known glycomer, which method comprises: (a) contacting a suitable sample from the subject with the first or second microarray, wherein the known glycomer is affixed at at least one discrete locus and wherein the contacting is performed under conditions which would permit the agent, if present in the sample, to specifically bind to the known glycomer in the microarray; and (b) determining whether the known glycomer in the microarray has the agent specifically bound thereto, thereby determining whether the subject is afflicted with the disorder.

The second method is a method of determining whether a subject is afflicted with a disorder characterized by the presence or absence in an afflicted subject of an agent which specifically binds to a known insoluble protein, which method comprises: (a) contacting a suitable sample from the subject with the first or second microarray, wherein the known insoluble protein is affixed at at least one discrete locus and wherein the contacting is performed under conditions which would permit the agent, if present in the sample, to specifically bind to the known insoluble protein in the microarray; and (b) determining whether the known insoluble protein in the microarray has the agent specifically bound thereto, thereby determining whether the subject is afflicted with the disorder.

The third method is a method of determining whether a subject is afflicted with a disorder characterized by the presence or absence in an afflicted subject of an agent which specifically binds to a known antibody or lectin, which method comprises: (a) contacting a suitable sample from the subject with the first or second microarray, wherein the known antibody or lectin is affixed at at least one discrete locus and wherein the contacting is performed under conditions which would permit the agent, if present in the sample, to specifically bind to the known antibody or lectin in the microarray; and (b) determining whether the known antibody or lectin in the microarray has the agent specifically bound thereto, thereby determining whether the subject is afflicted with the disorder.

In one embodiment of the instant diagnostic methods, the subject is human. In one embodiment of the first method, the disorder is an inflammatory disorder. In another embodiment of the first method, the inflammatory disorder is celiac disease. In one embodiment of the third method, the disorder is HIV-1 infection.

The following are specific examples of the instant diagnostic methods. In the first example, a subject's serum is analyzed for the presence of HIV-1 gp120 and IgG-anti-HIV-1 gp120, the presence of both indicating active HIV-1 infection. In the second example, a subject's serum is analyzed for the presence of either HIV-1 gp120 and IgG-anti-HIV-1 gp120, the absence of the

HIV-1 gp120 and the presence of IgG-anti-HIV-1 gp120 antibody indicating HIV-1 infection or immunization. In the third example, a subject's serum is analyzed for the presence of HIV-1 gp120 and IgG-anti-HIV-1 gp120, the absence of both indicating that the subject is neither HIV-1 infected nor immunized. In the fourth example, a subject's serum is analyzed for the presence of IgA-anti-gliadin and IgA-anti-TGt, the presence of both indicating that the subject is afflicted with celiac disease. Finally, in the fifth example, a subject's serum is analyzed for the presence of IgA-anti-gliadin, the presence of this antibody indicating the possibility that the subject is afflicted with celiac disease.

This invention further provides a method of determining whether an antibody known to specifically bind to a first glycomer also specifically binds to a second glycomer, which method comprises: (a) contacting the antibody with the first or second microarray, wherein a plurality of glycomers, other than the first glycomer, are affixed at discrete loci in the microarray, and wherein the contacting is performed under conditions which would permit the antibody to specifically bind to the first glycomer if it were present in the microarray; and (b) determining whether any of the glycomers in the microarray, other than the first glycomer, has the antibody specifically bound thereto, thereby determining whether the antibody also specifically binds to a second glycomer.

This invention further provides a method of determining whether an antibody known to specifically bind to a first insoluble protein also specifically binds to a second insoluble protein, which method comprises: (a) contacting the antibody with the first or second microarray, wherein a plurality of insoluble proteins, other than the first insoluble protein, are affixed at discrete loci in the microarray, and wherein the contacting is performed under conditions which would permit the antibody to specifically bind to the first insoluble protein if it were present in the microarray; and (b) determining whether any of the insoluble proteins in the microarray, other than the first insoluble protein, has the antibody specifically bound thereto, thereby determining whether the antibody also specifically binds to a second insoluble protein.

This invention further provides a method of making a microarray comprising a nitrocellulose or Hydrogel support having affixed to its surface at discrete loci a plurality of compounds, which method comprises contacting the nitrocellulose or Hydrogel support with the compounds under suitable conditions, whereby (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

This invention further provides a method of making a microarray comprising a plurality of nitrocellulose or Hydrogel supports, each support having one or a plurality of compounds affixed to its surface at a single discrete locus or a plurality of compounds affixed to its surface at discrete loci, which method comprises contacting the nitrocellulose or Hydrogel supports with the compounds under suitable conditions, whereby (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

This invention further provides a method of making the first article comprising contacting a nitrocellulose or Hydrogel support with dextran at discrete loci under suitable conditions.

In one embodiment of this method, the method further comprises the step of affixing at least one compound to the dextran at each discrete locus, whereby the composition of compounds at each discrete locus differs from the composition of compounds at at least one other discrete locus.

This invention further provides a method of making the second article comprising contacting a plurality of nitrocellulose or Hydrogel supports with dextran, whereby each support has dextran affixed to its surface at one or more discrete loci.

In one embodiment of this method, the method further comprises the step of affixing at least one compound to the dextran at each discrete locus, whereby the composition of compounds at each discrete locus differs from the composition of compounds at at least one other discrete locus.

This invention further provides six kits. The first kit comprises one of the instant microarrays and instructions for use. The second kit comprises one of the instant microarrays and a desiccant. The third kit comprises one of the instant microarrays immersed in an aqueous solution.

The fourth kit is a kit for practicing the first diagnostic method, which comprises: (a) a microarray comprising a nitrocellulose or Hydrogel support having affixed to its surface at discrete loci a plurality of compounds, wherein (i) at at least one discrete locus is affixed the glycomer to which the agent present or absent in an afflicted subject specifically binds, and (ii) the composition of compounds at each discrete locus differs from the composition of compounds at at least one other discrete locus; and (b) instructions for use.

The fifth kit is a kit for practicing the second diagnostic method, which comprises: (a) a microarray comprising a nitrocellulose or Hydrogel support having affixed to its surface at discrete loci a plurality of compounds, wherein (i) at at least one discrete locus is affixed the insoluble protein to which the agent present or absent in an afflicted subject specifically binds, and (ii) the composition of compounds at each discrete locus differs from the composition of compounds at at least one other discrete locus; and (b) instructions for use.

The sixth kit is a kit for practicing the third diagnostic method, which comprises: (a) a microarray comprising a nitrocellulose or Hydrogel support having affixed to its surface at discrete loci a plurality of compounds, wherein (i) at at least one discrete locus is affixed the antibody or lectin to which theagent present or absent in an afflicted subject specifically binds, and (ii) the composition of compounds at each discrete locus differs from the composition of compounds at at least one other discrete locus; and (b) instructions for use.

This invention further provides a first antibody capable of specifically binding to a glycomer present on the surface of a mammalian macrophage, which glycomer, or structural mimic thereof, is also endogenous to, and present on the surface of, a bacterial cell. In one embodiment, the antibody is a groove-type antibody. In another embodiment, the antibody is designated 4.3. F1 (ATCC Accession No. PTA-3259). In a further embodiment, the antibody is designated 45.21.1 (ATCC Accession No. PTA-3260).

This invention further provides a second antibody capable of specifically binding to a glycomer present on the surface of a mammalian intestinal epithelial cell, which glycomer, or structural mimic thereof, is also endogenous to, and present on the surface of, a bacterial cell. In one embodiment, the antibody is a cavity-type antibody. In another embodiment, the antibody is designated 16.4.12E (ATCC Accession No. PTA-3261).

This invention further provides a method of determining whether a subject is afflicted with a disorder characterized by the presence of a glycomer on the surface of macrophages in an afflicted subject, which glycomer, or structural mimic thereof, is also endogenous to, and present on the surface of, a bacterial cell, comprising: (a) contacting a sample of the subject's macrophages with the first antibody; and (b) determining whether the antibody specifically binds to the macrophages in the sample, such binding indicating that the subject is afflicted with the disorder. In one embodiment the subject is human. In another embodiment, the disorder is an immune disorder or an inflammatory disorder.

Finally, this invention provides a method of determining whether a subject is afflicted with a disorder characterized by the presence of a glycomer on the surface of intestinal epithelial cells in an afflicted subject, which glycomer, or structural mimic thereof, is also endogenous to, and present on the surface of, a bacterial cell, comprising: (a) contacting a sample of the subject's intestinal epithelial cells with the second antibody; and (b) determining whether the antibody specifically binds to the intestinal epithelial cells in the sample, such binding indicating that the subject is afflicted with the disorder. In one embodiment the subject is human. In another embodiment, the disorder is an immune disorder or an inflammatory disorder. In a further embodiment, the disorder is celiac disease.

This invention will be better understood from the Experimental Details that follow. However, one skilled in the art will readily appreciate that the specific methods and results discussed are merely illustrative of the invention as described more fully in the claims which follow thereafter.

### EXPERIMENTAL DETAILS

### First Series of Experiments

This invention provides novel antigen- and antibody-based microarrays for monitoring and quantifying a broad spectrum of biological molecules and their molecular interactions. A microarray technique is used to spot thousands of antigens and/or antibodies on a solid surface. This strategy can be applied to any molecular target, including naturally occurring proteins, carbohydrates, lipids and nucleic acids, as well as synthetic compounds. The instant microarray is useful for monitoring the expression of specific antibodies and other cellular factors in body fluids, and is therefore useful for disease diagnosis and basic immunological investigation. When a large repertoire of distinct monoclonal antibodies are arrayed, an antibody library microarray is produced. Application of these microarrays for global analysis of gene expression at the translational and post-translational levels is envisioned. The Elvin A. Kabat Collection of antigens and antibodies at Columbia University is useful in practicing this technology.

I. Materials, Methods and Results

(A) Method for Antigen/Antibody Immobilization

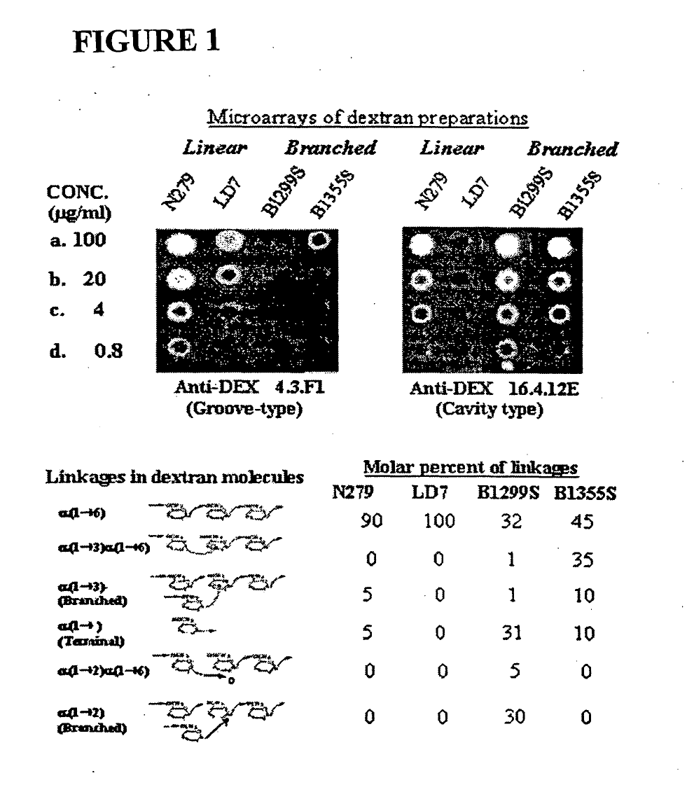

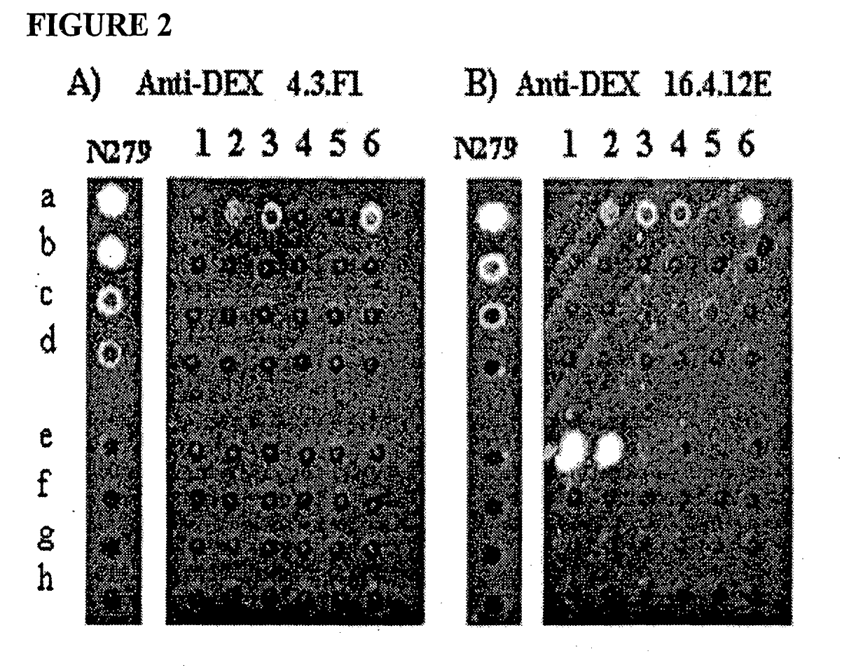

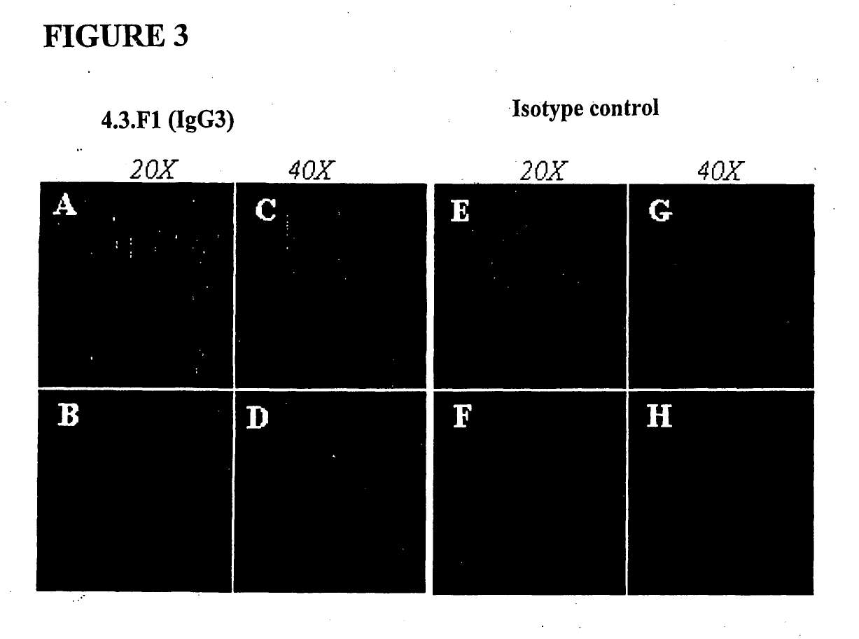

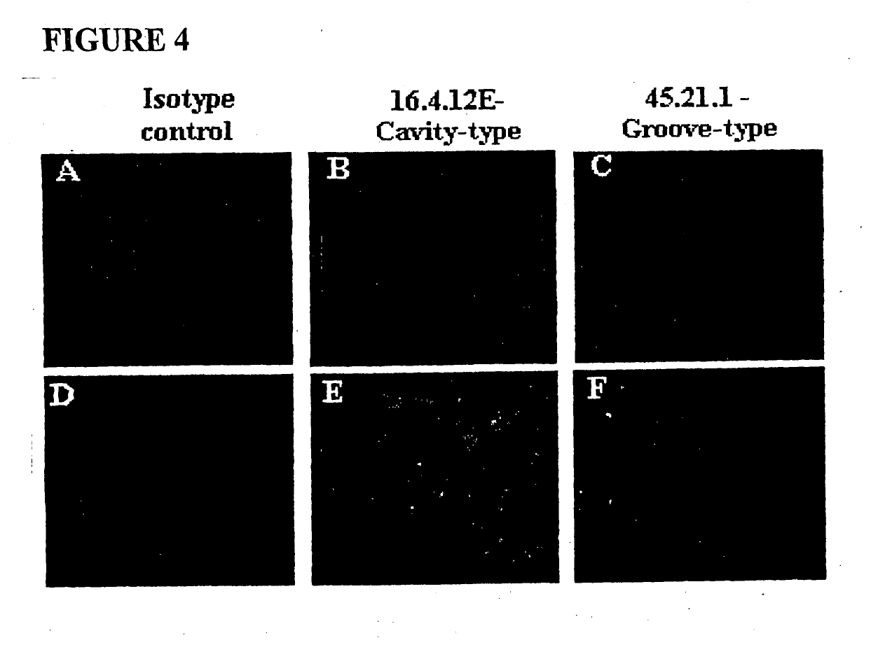

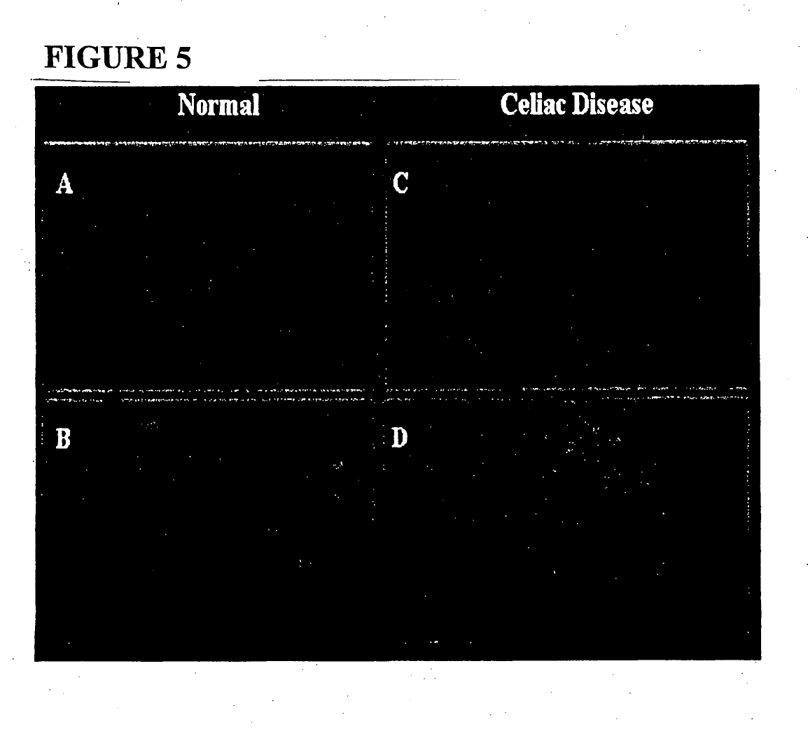

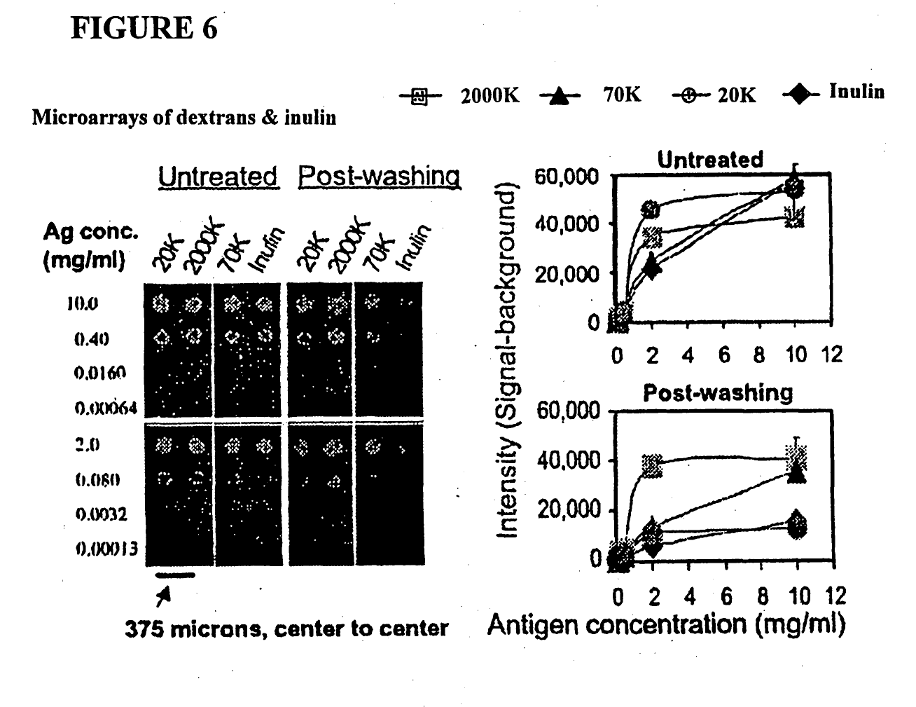

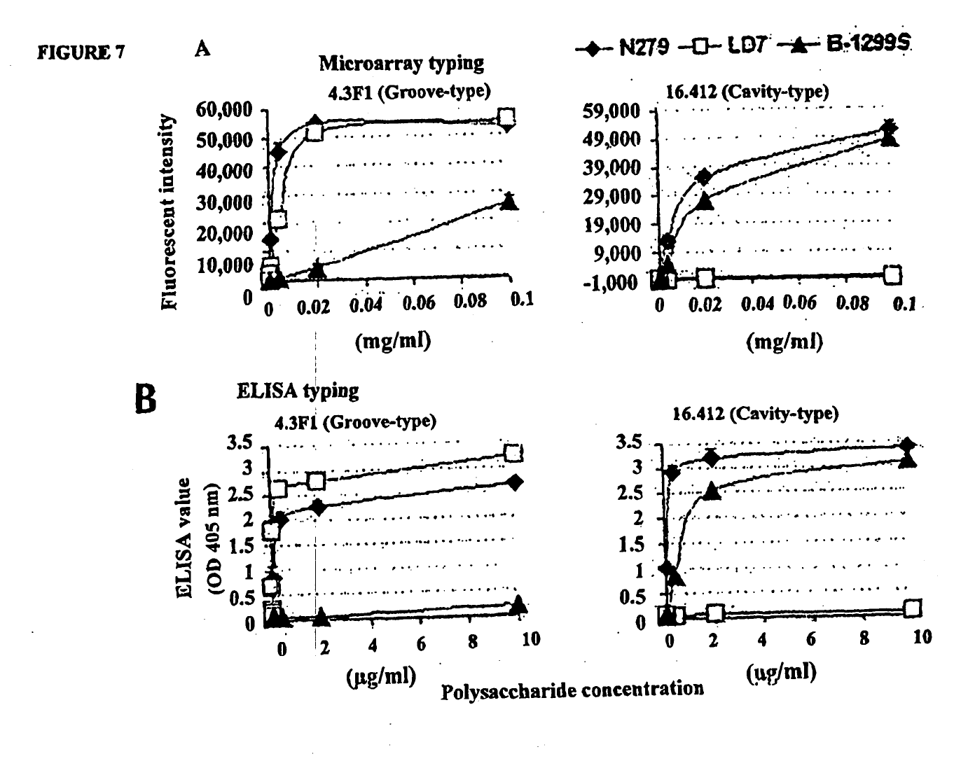

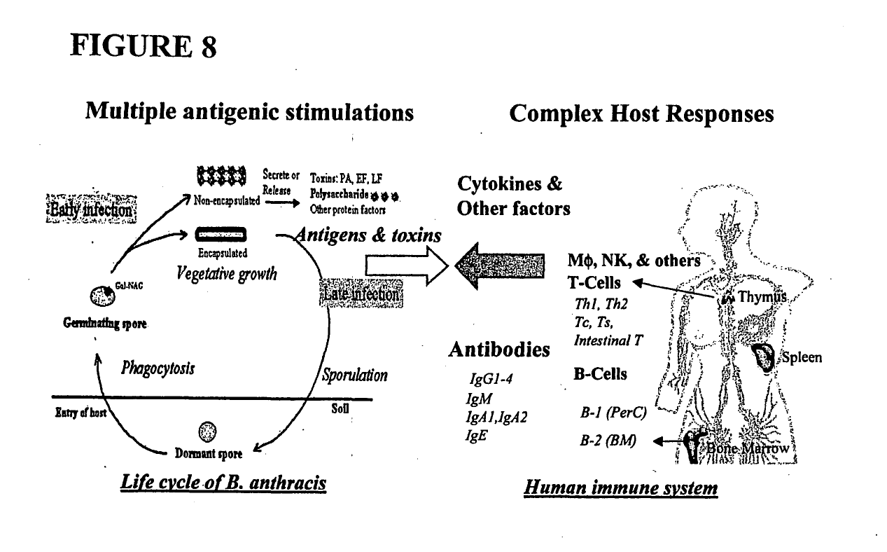

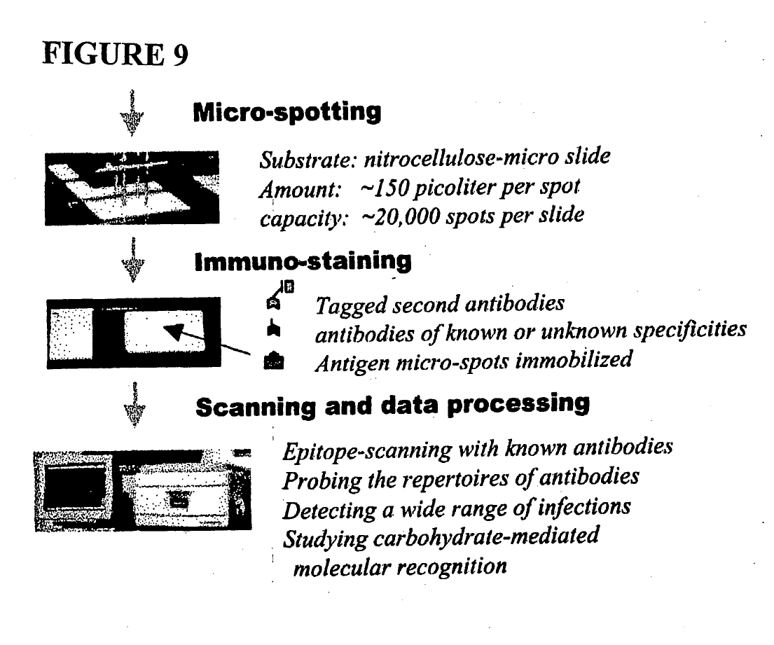

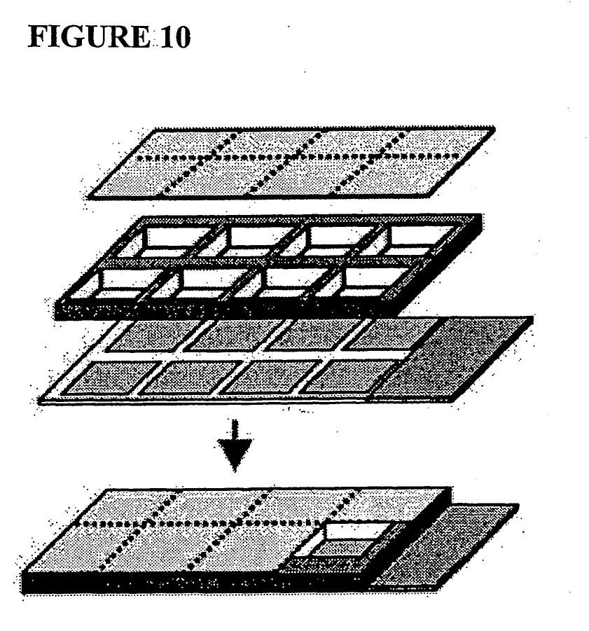

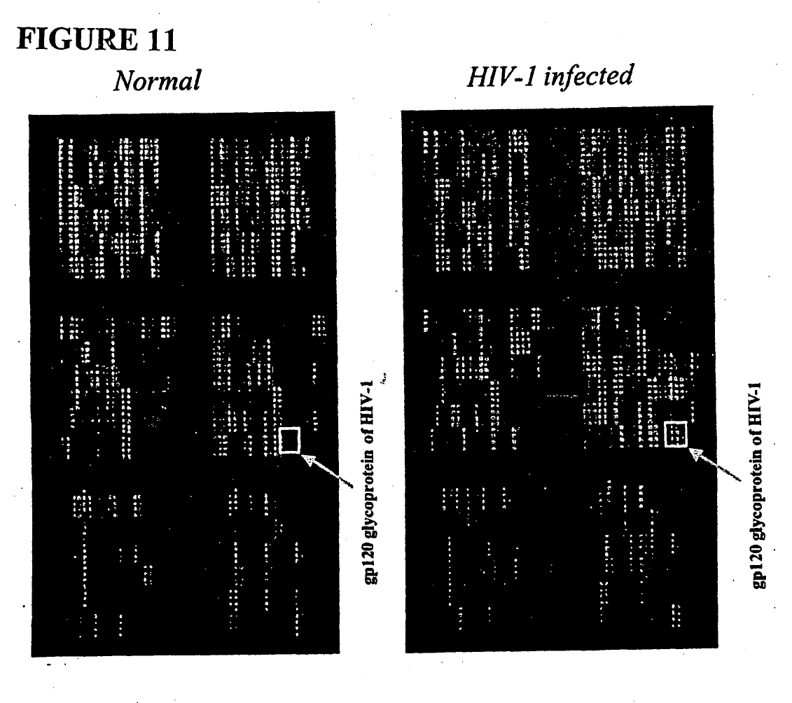

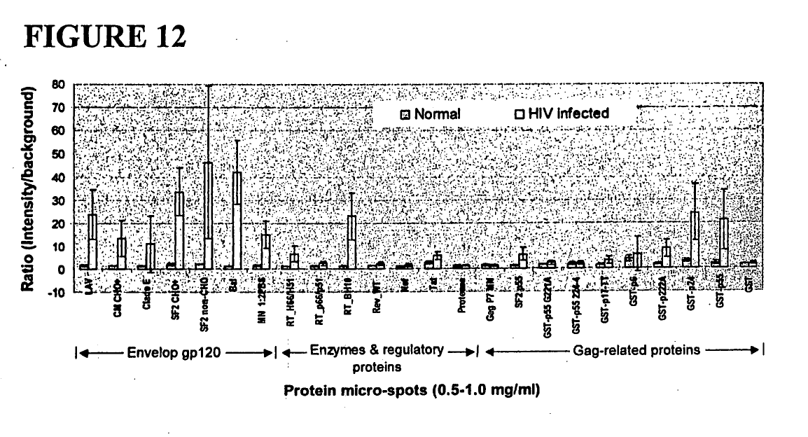

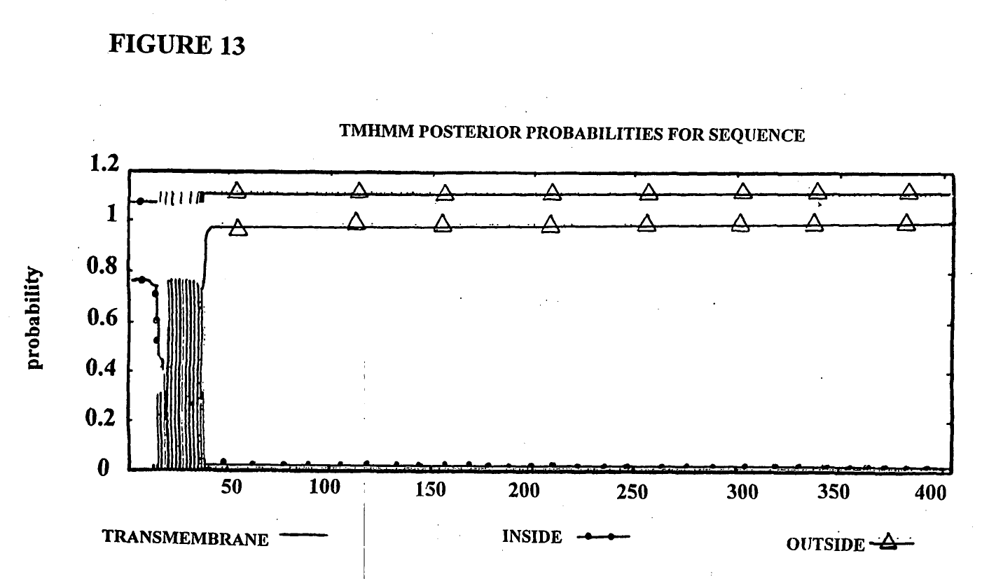

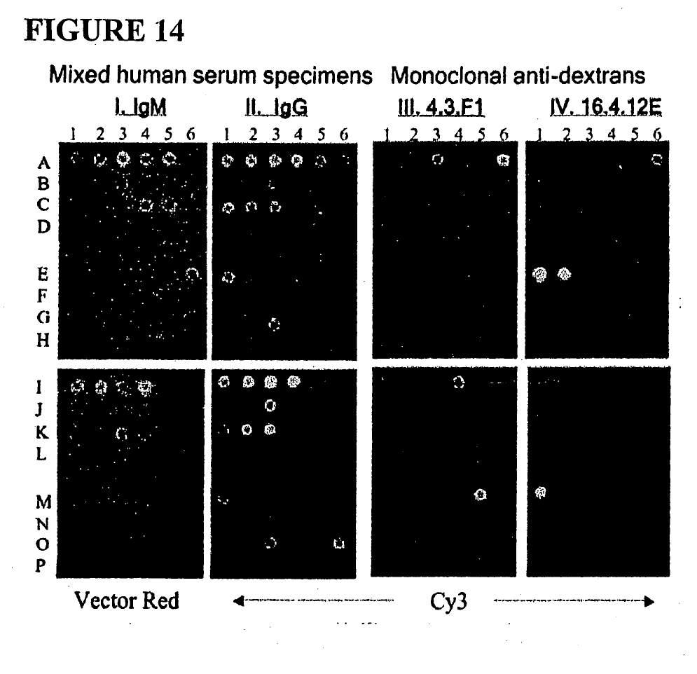

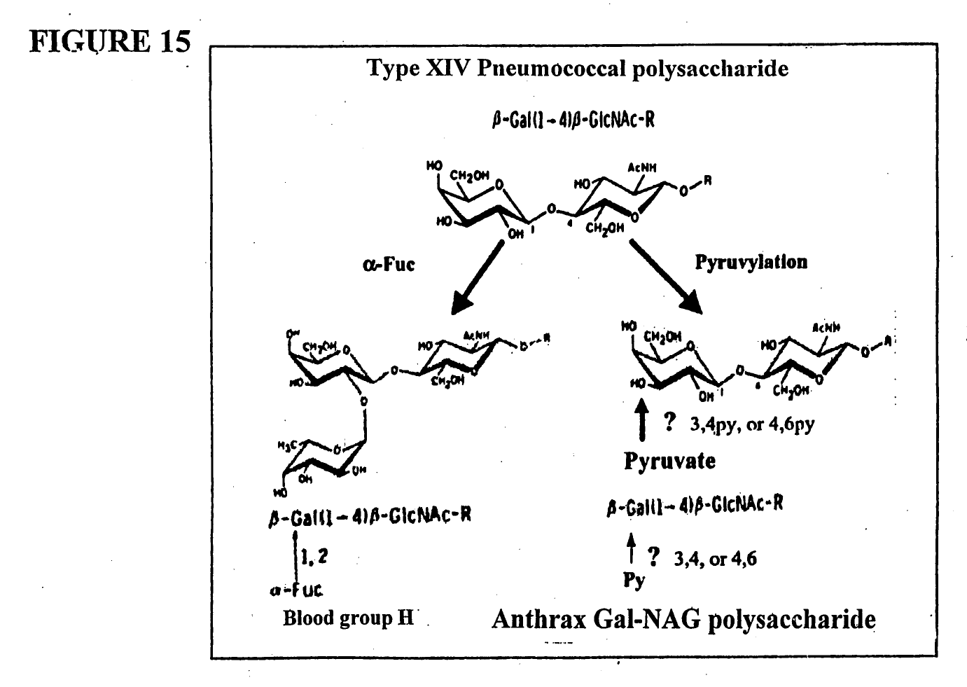

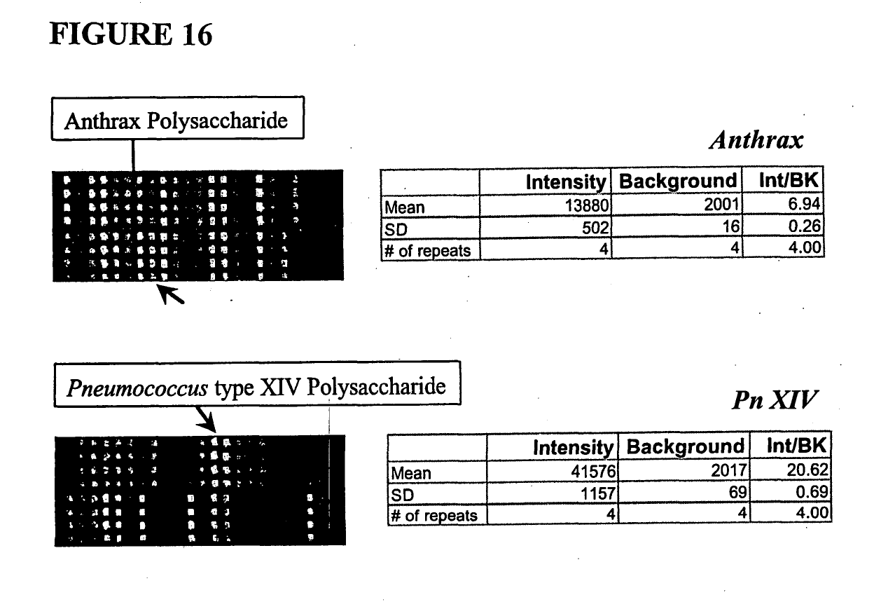

It is experimentally demonstrated here that nitrocellulose-coating can serve as a suitable matrix for immobilizing polysaccharides, glycoprotein, glycolipid, protein and antibodies on a glass surface without chemical conjugation. A set of commercially available glass slides, including those coated with nitrocellulose (ONCYTE Film-Slides, Grace Bio-Labs, Inc., Bend, Oreg.), poly-L-lysine (POLY-PREP™, Sigma), aminoalkylsilane (SILANE-PREPT™, Sigma) and regular micro-glass slides, were compared for their capacity to immobilize macromolecules of distinct structural properties. In the initial experiments, fluorescence-conjugated dextran molecules were applied. An extended panel of antigen preparations, including polysaccharide, glycoprotein, glycolipid, protein and antibody, were then investigated. Examples of these investigations are shown in FIGS. 1 and 2.

(B) Printing and Long-Term Storage of Antigen/Antibody Microarrays

A high-precision robot designed to produce CDNA microarrays (GMS 417 Arrayer, Genetic Microsystems, Inc., Woburn MA) was used to spot carbohydrate antigens onto a glass slide pre-coated with nitrocellulose polymer (ONCYTE Film-Slides, Grace Bio-Labs, Inc., Bend, Oreg.). Spots of antigens were printed with spot sizes at approximately 200 micron and 400 micron intervals, center-to-center. They were air dried and stored at room temperature before use. Conditions for long-term storage of the printed antigen/antibody microarrays were compared. The results show that (1) the air-dried carbohydrate microarrays can be stored at room temperature for at least one year without significant inactivation of their immunological activities; and (2) the antibody microarrays can be stored in an aqueous solution at 4° C. for at least one year without significant decrease in their antigen-binding activity, as illustrated using antibodies with anti-carbohydrate specificity.

(C) Proper Macromolecules for Coupling Smaller Bioreactive Molecules and Displaying them on a Solvent-Accessible Surface

Dextran preparations, especially α(1,6)dextrans, can serve as carrier molecules to conjugate other biologically active molecules. Such dextran-containing conjugates can be immobilized on a nitrocellulose-surface without further chemical conjugation. This method was demonstrated by immobilizing the fluorescence-α(1,6)dextran conjugates of different molecular weights, ranging from 35 kD to 2000 kD, on a nitrocellulose-coated glass slide. The fluorescence group is accessible to the anti-fluorescence antibodies in solution, thereby demonstrating specific binding. Methods for producing dextran-conjugates for surface immobilization follow.

(1) Mild Sodium Periodate Oxidization of Dextran to Create Highly Reactive Aldehyde Functional Groups (CHO)

α(1,6)dextran, preparation N279 (B512), was dissolved in 0.01M NaAcetate buffer, pH 5.5, at 10 mg/ml and warmed in a 37° C. water bath for 30 minutes. NaIO₄ was then added to the final concentration of 1×10⁻² M. The solution was mixed well and left to stand at room temperature for one hour in the dark. The preparation was dialyzed against 0.02M BBS (Borate buffered saline) pH 8.0, 4° C., overnight.

(2) Mild Oxidization of Carbohydrate Structure of IgG to Create CHO Group for Surface Immobilization

The above protocol was also applied to produce active CHO groups in carbohydrate molecules that exist naturally in the C region of IgG molecules. Such CHO-activated IgG is then covalently linked to the amino group of an amino-dextran molecule that was immobilized on a nitrocellulose-coated glass slide. Preparations of amino-dextrans are commercially available (Molecular Probes, Eugene). This method of IgG immobilization uses a carbohydrate structure attached in the Fc region of IgG, which is away from the antibody combining sites of antibody molecule, and thus preserves the antibodies' binding activity.

(3) Coupling of Biotin-LC-Hydrazide to the Oxidized Dextran

The above oxidized dextran was diluted 10-fold in 0.1 M NaAcetate, pH 5.5. A ⅓ volume of 5 mM Biotin-LC-Hydrazide was added dropwise. The mixture was shaken at room temperature for one hour. The reaction was terminated by addition of 0.5 ml of 1 M Tris HC1, pH 7.5, and the mixture was then dialyzed against Tris buffer (0.1 M Tris pH 7.5, 0.1 M NaCl, 2.0 mM MgCl2). The biotinylated dextrans are then ready to be immobilized on nitrocellulose-coated glass slides so that their biotin-groups are accessible to other molecules in solution.

(4) Surface Immobilization of Biotinylated Molecules

Standard methods were applied to couple NHS-Biotin (BRL #5533LA) to target molecules, i.e., either protein or polysaccharide. The biotinylated molecule was incubated with avidin at a proper molar ratio depending on the molecular weight of the target and its molar ratio with biotin. The molecules were then spotted on a surface that was precoated with biotin-dextran and blocked with BSA or gelatin. This strategy allows a flexible arrangement of antigen or antibody microarrays for a desired purpose, and avoids non-specific binding of target molecules on a surface.

(5) Glutaraldehyde-Conjugation to Generate Dextran-Small Molecule Conjugates

Small bioactive molecules containing amine group(s) can be coupled to amino-dextrans (Molecular Probes) by glutaraldehyde. Glutaraldehyde was added to the mixtures of the target molecule and amino-dextran (at proper molar ratios) to a final concentration of 0.2%. They were at room temperature for two hours. The reaction was stopped by addition of 1 M ethanolamine at 6.1 μl/ml. The mixture was at room temperature for an additional two hours and then dialyzed against 1X PBS or other proper solution, overnight. The dextran-small molecule conjugates were then spotted on nitrocellulose-coated slides. This method is suitable for generating microarrays having a large repertoire of small molecules and useful for high throughput drug screening or other biomedical investigations.

(D) Solutions for Preparing Macromolecules of Distinct Physicochemical Properties

1) Solution Storage

Microbial polysaccharides, blood group substances and glycolipids were solubilized and stored in saline at a concentration of about 1 mg/ml at 4° C. A small droplet of chloroform was added to prevent microbes. In this simple way, most solutions can be stored for years. The agents were diluted in saline at required concentrations immediately before spotting.

(2) Protein Antigens

Soluble protein preparations were prepared at relatively high concentrations in 1X PBS (mg/ml) with addition of 20% glycerol and frozen at −80° C. They were diluted in 1X PBS before use and stored at 4° C. for a short period of time (a few days). Antibody preparations were generally stored at 4° C. in 1X PBS except in special cases. Some E. Coli-expressed protein antigens are water-insoluble. The preparations were purified in a denatured condition and stored them in the same solution at 4° C. The freezing process is avoided for these proteins. In most cases these preparations can be immobilized on a nitrocellulose matrix without special treatment. This method has been applied successfully in applicant's laboratory for hybridoma screening.

(E) Staining and Scanning of Antigen/Antibody Microarrays

(1) General Application Protocol

Immediately before use, the printed antigen/antibody microarrays were rinsed with 1X PBS with 0.05% Tween 20 and then blocked by incubating the slides in 1% BSA in PBS containing 0.05% NaN₃ at 37° C. for 30 minutes. The microarrays were then incubated at room temperature with fluorescent-antibody conjugate at proper titration in 1% BSA PBS containing 0.05% NaN₃ and 0.05% Tween 20. Slides were rinsed with 1X PBS with 0.05% Tween 20 five times, air-dried at room temperature and then scanned for fluorescent signals. A ScanArray 5000 Standard Biochip Scanning System (GSI Lumonics, Inc. and Packard BioChip Technologies, Inc.) which is equipped with multiple lasers, emission filters, ScanArray Acquisition Software and QuantArray Microarray Analysis Software, was used to scan the stained antigen microarray, quantify spot-associated fluorescent-signals and analyze data.

(2) Special Application Protocols

(a) Method for Improving Signal Detection

A technical problem that has limited the application of the nitrocellulose-coated glass slide is its association with “white color” and non-specific fluorescent signals upon scanning. This problem is solved using the method that follows. (a) After staining a microarray glass slide, allow it to air-dry for a few minutes (at this stage, the nitrocellulose-coated region is white in color). (b) Soak the slide in 100% ethanol for 1-2 minutes until the white color of the nitrocellulose-coated region disappears and the entire slide becomes transparent. (c) Quickly spin the slide to remove extra ethanol. (d) Scan the slide when it is completely transparent. After storage for a few days or longer, depending on the humidity level in the air, the slide may turn back to the white color. One may repeat the above process to make the slide transparent again if necessary.

(b) Staining Antigen/Antibody Microarrays Using Non-Fluorescent Dyes

The antigen/antibody microarray can be stained with antibodies, antigens or other reactors that are conjugated with non-fluorescent dyes. The commonly used alkaline phosphatase (AP) and peroxidase are useful alternatives. One may practice this method as follows. (a) Stain an antigen microarray with a human serum sample at the proper dilution as described above. (b) After washing, stain the slide with an AP-conjugated anti-human IgG antibody at the proper dilution. (c) Wash the slide and develop the color by adding AP substrate BCIP alone or BCIP plus NBT. (d) Stop the reaction by adding a Tris-EDTA solution (20 mM Tris, pH 7.5, 5 mM EDTA). (e) Rinse the slide with distilled water and then scan it with a non-flourescent slide scanner, or observe the color reaction under a regular microscope.

(F) Sensitivity of Antigen Microarrays

Sensitivity is one of the critical parameters that determine the diagnostic value of antigen microarrays. In preliminary experiments (FIG. 1), dextran preparations were arrayed on nitrocellulose-coated glass slides in concentrations ranging from 100 μg/ml to 3.3 ng/ml. They were stained with fluorescence-labeled anti-dextran mAbs, either 4.3. F1 or 16.4.12E, at a concentration of 1 μg/ml. In both cases, the captured fluorescent signals (intensities) detected were positively correlated to the antigen concentrations. For example, 4.3. F1 was only detectable when the concentrations of N279, LD7 and B1299S are higher than 0.4, 20 or 100 μg/ml, respectively (spots of antigens with concentrations lower than 0.4 μg/ml were not shown in FIG. 1). Signal saturation was not observed even at the highest concentration of 100 μg/ml, showing the technical potential for improving the sensitivity of antigen microarrays.

(G) Epitope-Specific Antigen Microarray

Glycoconjugate technology was used to produce an “epitope-specific antigen microarray”. A naturally occurring antigen may be composed of multiple antigenic determinants. Frequently, one or a few of them serve as predominant antigenic determinants for host recognition. Antibodies specific for some carbohydrate epitopes of a microbial polysaccharide can be more protective than those reacting with others. There are also cross-reactive antigenic determinants that may be shared among strains or even species of microorganisms. Such cross-reactivities may cause difficulty in typing corresponding infectious agents. Thus, it is useful to define the fine specificities of antibodies elicited by an infection or by vaccination.

To produce an epitope-specific microarray, preparations of glycoproteins displaying α(1,6)-linked glucoses, i.e., isomaltotriose-coupled BSA (IM3-BSA) or its KLH-conjugate (IM3-KLH), were applied in a microarray experiment. These conjugates have the terminal non-reducing end epitope of α(1,6)dextran in common but differ in their protein carriers. As expected, mAb 16.4.12E (cavity-type), but not mAb 4.3. F1 (groove-type), bind to the microspots of IM3-protein conjugates (FIGS. 1E and 2E). Thus, glycoproteins can be immobilized on a nitrocellulose-coated glass slide and their antigenic determinants remain accessible to antibodies in solution.

Neoglycolipids, i.e., glycolipid conjugates, were used to produce epitope-specific microarrays, wherein stearylamine-isomaltosyl oligosaccharide conjugates, ST-IM3, ST-IM5 and ST-IM7, were applied (data not shown). Such glycolipid conjugates are homogeneic, since each lipid molecule can only be coupled by a single oligosaccharide. Unlike a glycoprotein conjugate, the sugar chain can be conjugated on to multiple sites of a protein molecule, generating a heterogenic population of antigenic determinants. Both sugar epitope and the amino acid residues adjacent to it may be structurally involved in forming these antigenic determinants.

(H) Antibody Microarrays

Experiments were performed to develop antibody-based microarrays. A panel of anti-dextran mAbs were immobilized at a concentration of 0.5 mg/ml on a set of glass slides. These included (a) a nitrocellulose-coated glass slide (ONCYTE Film-Slides, Grace Bio-Labs, Inc., Bend, Oreg.); (b) a poly-lysine treated slide (POLY-PREP™, Sigma); (c) a silane-treated glass slide (SILANE-PREP™, Sigma); and (d) an un-treated, pre-cleaned glass slide. These slides were then reacted with fluorescence-tagged dextran preparations of distinct structures. Only the nitrocellulose-slides showed spots of specific fluorescent signals. Thus, anti-dextran mAbs arrayed on the nitrocellulose-glass slide retained their antigen binding specificities. As described above, the antibody micrdarrays can be stored at room temperature in an air-dried condition for a few months and maintain their antibody-binding activity. The antibodies investigated include monoclonal antibodies of different isotypes (IgM, IgG and IgA).

II. Application of Antigen-Antibody Biochip Technology for Basic Research and Clinical Investigation

(A) Mapping Antibody Combining Sites by Applying Antigen/Antibody Microarrays

A well-established antigen-antibody system, i.e., dextrans and anti-dextran monoclonal antibodies (mAbs) [10, 11], was used for these investigations. A panel of purified dextran preparations of different linkage compositions and of different ratios of terminal/internal epitopes [10] were immobilized on nitrocellulose-coated glass slides and then incubated with monoclonal antibodies of defined specificities, either the cavity-type or the groove-type anti-dextrans [12]. The former is specific for the terminal non-reducing end structure of α(1,6)dextran; the latter recognizes the internal linear chain of the polysaccharide. When a cavity-type mAb, 16.4.12E, was applied on the glass slide, it bound to the immobilized α(1,6)dextran preparations having branches, but not those with only internal linear chain structures. By contrast, a groove-type mAb, 4.3. F1, bound to the dextran preparations dominated by linear chain structures, but not to the heavily branched α(1,6)dextrans (FIG. 1). Thus, the use of polysaccharides of defined structure in microarrays is an important strategy for studying antibody specificities and for characterizing immune responses to microbial infections.

As described above, methods to produce epitope-specific microarrays were also established, allowing characterization of the fine specificity of antibodies. Considering the presence of both the internal chain epitopes and the terminal non-reducing end epitopes in many polysaccharides, application of both polysaccharide macromolecules and their oligosaccharide conjugates on the instant microarrays will significantly enhance the power of the system, including its sensitivity, specificity and the detecting repertoires of antigenic determinants.

(B) Investigation of the Specificity and Cross-Reactivity of Antibodies/Receptors

An advantage of the instant microarray is that it provides a high throughput strategy to characterize the specificity and cross-reactivity of an antibody or a lectin molecule. FIG. 2 shows an example of this approach. A collection of about 50 carbohydrate-containing antigens, including microbial polysaccharides, blood group substances and other glyco-conjugates, were arrayed on the nitrocellulose-coated slide. They were then stained with anti-dextran mAbs, 4.3. F1, or 16.4.12E. As shown in FIG. 2, certain cross-reactive signals were detected for a few antigen preparations, being spots 2a, 3a and 6a for mAb 4.3. F1, and 2a, 3a, 4a, 6a, 1e, and 2e for 16.4.12E. Antigens arrayed at these locations are: 2a. Klebsiella polysaccharide type 11; 3a. Klebsiella type 13; 4a. Klebsiella type 21; 6a. Chondroitin sulfate B polysaccharide; 1e. IM3-BSA and 2e. IM3-KLH.

As discussed above, the binding of 16.4.12E (cavity-type), but not 4.3. F1 (groove-type), to IM3-BSA (1e) and IM3-KLH (2e) reflects the epitope-specific binding activities of these two monoclonal antibodies. Their binding to other antigens is, however, unexpected. The fluorescent intensities of cross-reactivities are much weaker than the binding to α(1,6)dextran N279. They were detected at a high antigen concentration (500 μg/ml), corresponding to or lower than the signals of specific bindings at much lower antigen concentrations (0.8 to 4 μg/ml). On an ELISA plate, such weak cross-reactivities are not detectable (data not shown). The cross-reactivities to CS-B polysaccharide (FIG. 2A and FIG. 2B), is interesting since CS-B is not a microbial antigen and was prepared from the intestinal mucosa of porcine. Further investigation of such reactivity led to the discovery of Dex-IdX as a cell type-specific marker in mouse and human (FIGS. 3-5).

(C) Clinical Application of Antigen Microarrays

The antigen-based microarray can be applied for detection and characterization of a wide range of microbial infections. During infection, whether viral, bacterial, fungal or parasitic, the host usually responds with the formation of antibodies which can be detected by a modified version of any of the methods used for antigen detection. The formation of antibodies and their time course depends on the antigenic stimulation provided by the infection. Recognition of these patterns provides evidence of recent or past infection. Microarrays of a large panel of antigens allow detection of many specificities in a single assay and thus allow a rapid diagnosis of infections. The diagnostic power of the instant microarrays will only increase as more microbial antigens and/or their antigenic determinants are characterized and applied.

To demonstrate this principle, a small scale antigen microarray with about 50 antigens was produced (FIG. 2) and tested with human sera from normal individuals and celiac patients. These specimens were diluted at 1:20 in 1% BSA-PBS with 0.025% Tween 20 and applied to the microarray. The bound human antibodies were visualized by application of a second antibody specific for human IgG or IgA that was conjugated with a fluorescent molecule (an anti-human IgG^{Cy3} and an anti-human IgA^{Cy5}. The positive staining of the microspots of these arrayed antigens by the serum specimens ranged from 6% (three of fifty spots were detected) to 12% (six of fifty spots were detected). The antigens detected were mainly microbial polysaccharides, including Klebsiella polysaccharide type 7, 13, 14, 21 and 33, Dudmans Rhizobium Trifelli TA1 and Levan.

The majority of the positive staining was of human IgG antibodies, although IgAs were also detected. This investigation has demonstrated that the instant microarray has the sensitivity to detect specific antibodies in human serum. Given the current capacity of microspotting technology in the art, about twenty thousand antigens can be arrayed on a single glass slide. It is expected, therefore, that this microarray is capable of characterizing a wide range of microbial infections using very limited samples in a single experiment.

III. Discussion

The instant microarrays are significantly different from known cDNA microarray and oligo-chip technologies, which target only nucleic acids. Applicant has employed high throughput microarray technology to develop a novel strategy for detecting, quantifying and characterizing proteins, carbohydrates and other biological molecules, and useful in the new areas of post-genomic research, namely proteomics and glycomics.

Differing from current immunoassays which detect specific molecules one-by-one, this technology is designed to detect and quantify a large repertoire of distinct biological molecules in a single assay. Combining a high throughput microarray technique and a sensitive confocal fluorescent scanning method, this technology is useful for detecting thousands of distinct molecules using a small amount of biological specimen, such as a drop of blood or other bodily fluid. This technology can be extended to the genome-wide scanning of protein expression and post-translational modification.

References

1. Ramsay, G., DNA chips: state-of-the art. Nature Biotechnology, 1998. 16(1): p. 40-4.

2. Brown, P. O. and D. Botstein, Exploring the new world of the genome with DNA microarrays. Nature Genetics, 1999. 21(1 Suppl): p. 33-7.

3. DeRisi, J. L., V. R. Iyer, and P. O. Brown, Exploring the metabolic and genetic control of gene expression on a genomic scale. Science, 1997. 278(5338): p. 680-6.

4. Lueking, A., et al., Protein microarrays for gene expression and antibody screening. Analytical Biochemistry, 1999. 270(1): p. 103-11.

5. Ge, H., UPA, a universal protein array system for quantitative detection of protein-protein, protein-DNA, protein-RNA and protein-ligand interactions. Nucleic Acids Research, 2000. 28(2): p. e3.

6. Varki, A., Biological roles of oligosaccharides: all of the theories are correct. Glycobiology, 1993. 3(2): p. 97-130.

7. Feizi, T., Carbohydrate recognition systems in innate immunity. Advances in Experimental Medicine & Biology, 1998. 435: p. 51-4.

8. Feizi, T. and R. W. Loveless, Carbohydrate recognition by Mycoplasma pneumoniae and pathologic consequences. American Journal of Respiratory & Critical Care Medicine, 1996. 154(4 Pt 2): p. S133-6.

9. Zareba, T. W., et al., Binding of extracellular matrix proteins by enterococci. Curr Microbiol, 1997. 34(1): p. 6-11.

10. Wang, D. and E. A. Kabat, Carbohydrate Antigens (Polysaccharides), in Structure of Antigens, M. H. V. V. Regenmortal, Editor. 1996, CRC Press: Boca Raton New York London Tokyo. p. 247-276.

11. Wang, D., et al., The repertoire of antibodies to a single antigenic determinant. Molecular Immunology, 1991. 28(12): p. 1387-97.

12. Cisar, J., et al., Binding properties of immunoglobulin combining sites specific for terminal or nonterminal antigenic determinants in dextran. J. Exp. Med., 1975. 142: p. 435-459.

13. Matsuda, T. and E. A. Kabat, Variable region cDNA sequences and antigen binding specificity of mouse monoclonal antibodies to isomaltosyl oligosaccharides coupled to proteins T-dependent analogues of α(1,6)dextran. J. Immunol., 1989. 142: p. 863-870.

14. Chen, H. T., S. D. Makover, and E. A. Kabat, Immunochemical studies on monoclonal antibodies to stearyl-isomaltotetraose from C58/J and a C57BL/10 nude mouse. Mol. Immunol., 1987. 24: p. 333-338.

15. Wang, D., et al., Two families of monoclonal antibodies to α(1,6)dextran, V_{H}19.1.2 and V_{H}9.14.7, show distinct patterns of Jₖ and J_{H} minigene usage and amino acid substitutions in CDR3. J. Immunol., 1990. 145: p. 3002-3010.

### Second Series of Experiments

I. Materials and Methods

A. Carbohydrate Antigens and Antibodies

Carbohydrate-containing macro-molecules applied in FIG. 7, and anti-α(1,6)dextran mAbs, 16.4.12E (IgA/kappa)(6), 4.3F1 (IgG3/kappa)(5), and 45.21.1 (IgA/kappa)(8), were adopted from the collection of the late Professor Elvin A. Kabat of Columbia University. Purified proteins of 4.3F1, 45.21.1 and 16.4.12E were obtained by a procedure of affinity purification as described(9). The biotinylated or FITC-conjugated anti-dextran antibodies were prepared in our laboratory following standard protocols(10). The FITC-conjugated α(1,6)dextrans of moleuclar weight 20 kDa, 70 kDa, and 2,000 kDa, FITC-inulin, a biotinylated anti-human IgG antibody, an alkaline phosphatase-conjugated anti-human IgM, and streptavidin conjugates, were purchased from Sigma (St. Louis, Mo.). Antibodies for cell type/lineage analysis, including antibodies specific for murine CD11b/MAC1, MAC3, TCR-α, TCR-β, CD3, CD4, CD5, CD8, CD19, B220, Syndecan-1, a mouse IgG3 isotype standard (A12-3), and a streptavidin conjugate of Texas Red, were from BD-PharMingen (San Diego, Calif.). A streptavidin-Cy3 conjugate was purchased from Amersham Pharmacia (Piscataway, N.J.), and a red fluorescence substrate of alkaline phosphatase, Vector Red, from Molecular Probes, Inc. (Burlingame, Calif.).

B. Printing Carbohydrate Microarrays

A high-precision robot designed to produce cDNA microarrays (GMS 417 Arrayer; Genetic Microsystems, Inc., Woburn, Mass.) was utilized to spot carbohydrate antigens onto the glass slides precoated with nitrocellulose polymer (FAST Slides; Schleicher & Schuell, Keene, N.H.). Carbohydrate antigens were dissolved in saline (0.9% NaCl) in concentrations as specified in the Figure legends. They were printed with spot sizes of ^{˜}150 μm and at 375-μm intervals, center to center. The printed carbohydrate microarrays were air-dried and stored at room temperature without desiccant before application.

C. Staining and Scanning of Carbohydrate Microarrays

Immediately before use, the printed carbohydrate microarrays were rinsed with PBS, pH 7.4, with 0.05% (vol/vol) Tween 20 and then blocked by incubating the slides in 1% (wt/vol) BSA in PBS containing 0.05% (wt/vol)NaN₃ at 370° C. for 30 minutes. They were then incubated at room temperature with antibodies at an indicated titration in 1% (wt/vol) BSA in PBS containing 0.05% (wt/vol) NaN₃ and 0.05% (vol/vol) Tween 20. Application of secondary antibodies or streptavidin conjugates is specified in figure legends. The stained slides were rinsed five times with PBS with 0.05%(vol/vol) Tween 20, air-dried at room temperature, and then scanned for fluorescent signals. The stained microarrays were scanned with a ScanArray 5000 Standard Biochip Scanning System (Packard BioChip Technologies, Inc., Billerica, Mass.) and data analyzed using Quant Array version 2.1 software associated with the system.

D. Elisa and in Situ Immunofluorescence

ELISA and immunofluorescence staining were carried out as described (6, 11). The dextranase treatments were performed by a preincubation of tissue sections with dextranase(Sigma) at 0.5 unit/ml in 100 mM potassium PBS, pH 6.0, 37° C. for 60 minutes. This condition allows a complete removal of molecules of FITC-α(1,6)dextran that were specifically trapped by immune cells in the spleen sections of α(1,6)dextran-immunized mice (11).

II. Results and Discussion A model system for establishing carbohydrate microarray technology. The dextrans and anti-dextran antibodies (1, 2) were applied to establish methods for immobilizing carbohydrate polymers on glass slides. Dextrans are polymers composed entirely of glucose, produced mainly by bacteria of the family Lactobacillaceae and of the genera Leuconostoc and Streptococcus. Dextran molecules derived from different strains may, however, differ significantly in their glycosidic linkage compositions. Unlike proteins that are linked solely by a peptide bond, carbohydrates utilize many possible glycosidic linkages so as to diversify their structures extensively. Some dextran preparations are predominantly or solely α(1,6)-linked, forming molecules with dominantly linear chain structures; others are composed of multiple glycosidic linkages, including α(1,6)-, α(1,3)-, α(1,2)-, and others, generating heavily branched molecules(3) (Table 1). Previous immunological studies (2, 4) have demonstrated that such structural characteristics are detectable by antibodies specific for different antigenic determinants or epitopes of dextran molecules. This system is, therefore, suitable for developing methods for immobilization of carbohydrate antigens and for investigating their immunological properties in a surface-immobilized configuration.

We applied the fluorescein isothiocyanate (FITC)-conjugated polysaccharides as probes to investigate whether nitrocellulose-coated glass slides can be used to immobilize microspots of carbohydrate polymers without covalent conjugation. FITC-α(1,6)dextran preparations of different molecular weights and a structurally distinct polysaccharide, inulin, were printed on the glass slides using a microprinting device to produce a carbohydrate microarray (FIG. 6A). Their fluorescent signals were then captured and quantified by a microscanning system that was developed for scanning complementary DNA (CDNA) microarrays. By analyzing the fluorescent intensities retained on the slides after extensive washing, we demonstrated that dextran preparations ranging from 20 kDa to 2,000 kDa and inulin of 3.3 kDa were all stably immobilized on the nitrocellulose-coated slide without chemical conjugation. The efficiency of their immobilization was, however, significantly influenced by the molecular weight. The larger dextran molecules were better retained than the smaller ones (FIG. 6A, B).

To investigate whether immobilized carbohydrate macromolecules preserve their antigenic determinants or epitopes, dextrtan preparations of different linkage compositions(3) and of different rations of terminal to internal epitopes (1, 4) were printed on nitrocellulose-coated glass slides. These preparations include N279, displaying both internal linear and terminal nonreducing end epitopes; B1299S, heavily branched and expressing predominantly terminal epitopes; and LD7, a synthetic dextran composed of 100% α(1,6)-linked internal linear chain structure. The dextran microarrays were incubated with monoclonal antibodies (mAbs) of defined specificities, either a groove-type anti-α(1,6)dextran 4.3F1 (IgG3) (5) or a cavity-type anti-α(1,6)dextran 16.412E (IgA) (6). The former recognizes the internal linear chain of α(1,6) destrans; the latter is specific for the terminal nonreducing end structure of the polysaccharide. As shown in FIG. 7A (left), the groove-type mAb, 4.3F1 (refs 5,7) bound to the dextran preparations with predominantly linear chain structures, N279 and LD7, but bound poorly to the heavily branched α(1,6)dextran, B1299S. By contrast, when the cavity-type mAb 16.4.12E (FIG. 7A, right) was applied, it bound to the immobilized dextran preparations having branches (N279 and B1299S) but not those with only internal linear chain structure (LD7). These patterns of antigen-antibody reactivities are characteristically identical to those recognized by an ELISA binding assay (FIG. 7B) and other classical quantitative immunoassays for either the groove-type (4, 5, 7) or the cavity type (4, 6) of anti-dextran mAbB. We conclude, therefore, that dextran molecules immobilized on a nitrocellulose-coated glass slide have their immunological properties well preserved. Both their nonreducing end structure, recognized by the cavity-type anti-α(1,6)dextrans, and the internal linear chain epitopes, bound by the groove-type anti-α(1,6) dextrans, are displayed on the surface after immobilization and are accessible to antibodies in an aqueous solution.

### References

1. Wang, D. & Kabat, E. A. Carbohydrate antigens (polysaccharides); Structure of Antigens, Vol. 3 (ed. M. H. V. V. Regenmortal) 247-276 (CRC Press, Boca Raton Fla.; 1996).

2. Wang, D. et al., The repertoire of antibodies to a single antigenic determinant. Mol. Immunol. 28, 1387-1397 (1991).

3. Jeanes, A. Immunochemical and related interactions with dextrans reviewed in terms of improved structural information. Mol. Immunol. 23, 999-1028 (1986).

4. Cisar, J., Kabat, E. A., Dörner, M. M. & Liao, J. Binding properties of immunoglobulin combining sites specific for terminal or nonterminal antigenic determinants in dextran. J. Exp. Med. 142, 435-459 (1975).

5. Wang, D. et al., Two families of monoclonal antibodies to α(1,6)dextran, V_{H}19.1.2 and V_{H}9.14.7, show distinct patterns of J_{K} and J_{H} minigene usage and amino acid substitutions in CDR3. J. Immunol. 145, 3002-3010 (1990).

6. Matsuda, T. & Kabat, E. A. Variable region cDNA sequences and antigen binding specificity of mouse monoclonal antibodies to isomaltosyl oligosaccharides coupled to proteins. T-dependent analogues of α(1,6)dextran. J. Immunol. 142, 863-870 (1989).

7. Chen, H. T., Makover, S. D. & Kabat, E. A. Immunochemical studies on monoclonal antibodies to stearyl-isomaltotetraose from C58/J and a C57BL/10 nude mouse. Mol. Immunol. 24, 333-338 (1987).

8. Sharon, J., Kabat, E. A. & Morrison, S. L. Association constants of hybridoma antibodies specific for α(1→6) linked dextran determined by affinity electrophoresis. Mol. Immunol. 19, 389-397 (1982).

9. Lai, E. & Kabat, E. A. Immunochemical studies of conjugates of isomaltosyl oligosaccharides to lipid: productiona nd characterization of mouse hybridoma antibodies specific for stearyl-isomaltosyl oligosaccharides. Mol. Immunol. 22, 1021-1037 (1985).

10. O'Shannessy, D. J., Dobersen, M. J. & Qarles, R. H. A novel procedure for labeling immunoglobulins by conjugation to oligosaccharide moieties. Immunol. Lett. 8, 273-277 (1984).

11. Wang, D., Wells, S. M., Stall, A. M. & Kabat, E. A. reaction of germinal centers in the T-cell-independent response tot he bacterial polysaccharide α(1-6)dextran. Proc. Natl. Acad. Sci. USA 91, 2502-2506 (1994).

### Third Series of Experiments

I. Introduction

A microbial infection may expose and release multiple antigenic substances to a host, eliciting a comprehensive host immune response, including a B cell response, which produces specific antibodies, and a T cell response, resulting in specific T cell activation and cytokine production. There is also an activation of macrophage, dendritic cells and other accessory cells, leading to production of differential profiles of cytokines and inflammation factors. Some microbial substances are lethal to a host upon their immediate release or after interacting with host cells or cellular factors. The interplay of different types of host cells and multiple protein factors and their interaction with the invading pathogen determine the progress and consequence of a microbial infection. For example, in an anthrax infection, the pathogen Bacilluus anthracis may expose and release a number of antigens of distinct structural characteristics to a host, eliciting a comprehensive picture of a host response (FIG. 8).

To better understand the pathogenesis mechanisms of an infectious disease, it is crucially important to monitor the full-spectrum of the multi-parameter host responses and identify the characteristic patterns of the response. Monitoring such a complex host response and host-microbe interaction has long been a challenge to biomedical scientists and clinicians. Many antigen-antibody binding assays are currently in use for clinical diagnosis of infectious and non-infectious diseases. These include the classical direct immunoassays, such as, immunodiffusion, immunoelectrophoresis, agglutination and immunoprecipitation, and recently developed methods, including immunofluorescence, radioimmunoassay (RIA), enzyme-immunoassay (EIA) and western blot. These approaches take advantage of the specificity of antigen-antibody interaction but are designed to operate on a one-by-one basis.

Rapid progress of the genome sequencing projects has led to the development of a generation of high throughput technologies for biological and medical research. These include the nucleic acid-based microarrays (6,7) or DNA chips (8), and the protein-based microarrays (9,10). Our recent efforts have been focusing on the development of a carbohydrate and protein-based microarray technology to extend the scope of biomedical research on carbohydrate-mediated molecular recognition and anti-infection responses (see reference 11 and next section for our most recent progress).

The microspot format of surface displaying biological molecules has the advantage of achieving a highly sensitive and simultaneous detection of multiple binding partners in solution. Since the amount of molecule in the solution phase that is required for saturating the surface immobilized microspots of molecules is considerably small, binding can be achieved with a relatively lower molar concentration of molecules in solution. In brief, it is believed that the smaller microspot is better than the bigger spot in its sensitivity of detection in an assay system (12,13).

(A) Category a Pathogens and Their Genomic Information

High-priority infectious agents that pose current risks to our national security include multiple microbial pathogens known as Category A pathogens, such as Bacillus anthracis (anthrax), Clostridium botulinum (botulism), Yersinia pestis (plague), Variola viruse (smallpox), Francisella tularensis (tularemia) and viral hemorrhagic fevers (Lassa virus, Ebola virus, Marburg virus and Lymphocytic Choriomeningitis virus) An assay system that allows detection and characterization of these pathogens in a single assay using limited clinical specimens is currently unavailable.

A great deal of genomic information is available for numerous pathogens (14-16), providing many opportunities to the field of infectious disease research. The identification of candidates from a large repertoire of genes, including a large panel of genes whose function is unknown, for developing diagnostic protein biochips is, however, a current challenge. As detailed below, we have established a strategy to facilitate the selection and identification of genes that have potential to serve as novel molecular targets for vaccination and diagnosis. Information provided by whole genome sequencing of microbes may also boost progress in the molecular engineering of artificial species of desired characteristics. There is also concern that the release of laboratory amplified genetic material to the environment may facilitate the natural occurrence of recombinant or newlyevolved microbial species or strains. Such emerging microbes could be either beneficial or harmful to public health depending on the nature of the microorganism as well as the way by which they are utilized. Therefore, in designing a high throughput immunoassay one must consider the detection of existing pathogens as well as their mutants or recombinant forms. This goal is achievable if a panel of antigens of a given microbe and/or their specific antibodies are applied to produce a diagnostic biochip to detect multiple molecular targets of the pathogen. Applicants have tested the feasibility of this proposal by printing a panel of HIV-1 proteins on a single chip to monitor the HIV-1 infections by different classes or clades of HIV-1 viruses (see FIGS. 11 and 12).

(B) Antigen-Based Biochip

An antigen biochip is designed to detect and quantify antibodies in body fluids. During infection, whether viral, bacterial, fungal, or parasitic, the host usually responds with formation of antibodies, which can be detected by modification of any of the methods used for antigen detection. The formation of antibodies and their time course depends on the antigenic stimulation provided by the infection. Recognition of these patterns provides us with evidence of recent or past infection.

Establishing a biochip to display a large panel of microbial antigens and the host derived autoantigens will substantially extend the. scope of biomedical research on the biological relationship between host and microorganism, as well as understanding the molecular mechanisms of infectious diseases. Both carbohydrate antigens and protein antigens can be of importance for the design and production of a diagnostic biochip.

Carbohydrate structures of microbial origin, including polysaccharides, glycolipids and glycoproteins, frequently serve as the main antigenic structures to which host cells recognize and mount a response (17). A single microbial antigen may, however, display multiple antigenic determinants, with one or a few of them predominating in a given infection. For example, a relatively simple microbial polysaccharidej α(1,6) dextran N279, displays both the internal linear chain and terminal non-reducing end structures as distinct antigenic determinants. When the polysaccharide was injected to a Balb/c mouse, it elicited a predominant antibody response directed to the internal linear chain epitope of α(1,6)dextran. The internal linear chain epitope is thus defined as a dominant antigenic determinant to the host. The antibodies elicited were solely or dominantly of the groove-type anti-dextran. In this case, the terminal non-reducing end structure is apparently the minor antigenic determinant. A dominant antigenic determinant should, therefore, be placed as a high priority to serve as a target for vaccine design or for developing an immunoassay for diagnosis.

Identifying a minor antigenic determinant is, however, also important since there is the possibility that a dominant antigenic structure is not suitable for vaccination or diagnostic application; whereas, a minor antigenic determinant may serve as a molecular target for these applications. Some microbial antigens may share or mimic host components. This is known as antigenic cross-reactivity. For examples, a(1→8)NeuNAc in the capsule of the group B meningococcus and of E.coli K1 is found on glycoproteins and gangliosides of human tissues, complicating the application of the capsular polysaccharides for vaccination, especially in infants who have expression of polymeric forms of the carbohydrate structure. There are also circumstances that a dominant antigenic structure can be useful for diagnosis but not for vaccination. For example, the Gag p24 protein of HIV-1 elicits a dominant antibody response in most AIDS patients. Detecting anti-Gag antibodies has diagnostic value. These antibodies, however, have no HIV-1 neutralization activity since Gag is not expressed on the surface of the HIV-1 virus. Even for the envelope protein, gp120 of HIV-1, which is surface displayed and accessible to antibody recognition, there are only a few epitopes of the glycoprotein to which antibodies can be effective in virus neutralization (18).

With current immunological advances, converting a minor antigenic determinant into a dominant one is technically achievable. For example, the antigen may be coupled with a carrier molecule, forming a highly antigenic conjugate molecule. As demonstrated with a model antigenic system α(1,6)dextran, an isomaltotriose (IM3), which is derived from the polysaccharide α(1,6)dextran, can be coupled to BSA or KLH to produce a semi-synthetic glycoprotein to display the minor antigenic determinant of the polysaccharide, i.e., the terminal non-reducing end epitopes of α(1,6)dextran. When this glycoconjugate was injected to Balb/c, a dominant antibody response to the terminal antigenic determinant is induced. Immunization with such an artificial semi-synthetic glycoconjugate is now well known as the conjugate vaccine. A notable example is vaccination with the protein-conjugates of Haemophilus influenza type b polysaccharide that resulted in the decline in the incidences of H. influenza meningitis and other infections in infants and children (19,20). The presence of polysaccharides and glycoproteins in Bacilluus anthracis has been recognized for some time (4, 21,22). Whether these carbohydrate structures are suitable targets for anthrax vaccination is, however, an open question.

Recognition of cross-reactive antigenic determinants is frequently shown to be of biological and medical significance. There are a number of documented cases in which microbial antigens mimic the structures of host components, assisting a microbe to escape from a host's immune defense (23,26). Such mimicking microbial antigens may also induce autoimmune disorders and contribute to the pathogenesis of an infectious disease (23,27). Identification and characterization of such antigenic structures may lead to a better understanding of the molecular mechanisms of infectious diseases.

Antigenic structures that are not expressed on the surface of a microorganism also have an important diagnostic value. For example, detection of antibodies that are specific for the surface antigen of hepatitis B (HBsAg) may indicate an early viral infection or a successful vaccination; detecting antibody specificities to multiple viral antigens, such as surface antigen (HBsAg) plus core antigen (HBcAg) or HBsAg plus a relevant e antigen (HBeAg) suggests an active infection or the progression of the disease (28,29). A microbial pathogen, either a virus or a bacterium, may release multiple antigenic substances that trigger host antibody responses. In an early infection, as an initial immune response, the antibodies elicited are mainly those bound to the surface antigens and are predominantly IgM antibodies. With the progression of an infectious disease, for example in an AIDS patient a few months post-serum conversion, a large panel of IgG antibodies with different specificities, including anti-gp120 (envelope protein) and anti-Gag p55 polyproteins of HIV-1, can be detected in the serum of the patient. Thus, applying a combination of surface and non-surface antigens on a biochip for diagnosis can assist in the recognition of the stages or steps of an infection and provide information to predict disease progression and to evaluate the efficacy of a therapeutic agent or strategy.

Development of drugs or therapeutic strategies against microbial infection requires a better understanding of the pathogenic mechanisms of an infectious disease. For example, the anthrax toxin is lethal upon its activation. This process involves multiple steps of molecular and cellular interactions of anthrax proteins, host cells and protein factors (1-3). The protective antigen of anthrax, named PA, is an integrated component of the lethal toxin of Bacilluus anthracis. It binds to a specific cellular receptor and forms toxic, cell bound complexes with edema factor (EF) and lethal factor (LF) (1-3). This understanding leads to the developmentof a polyvalent inhibitor-based therapeutic strategy to protect a host from lethal attack by the toxin (5). Technically, it is of crucial importance to identify the structural moieties or epitopes that play key roles in forming the toxic complex or in the interaction of PA and its macrophage receptor. Generally speaking, the epitopes of a protein or a glycomer that interact with or are recognized by their partners are surface exposed and are, therefore, recognizable by specific antibodies. Identifying such antigen-antibody pairs is of crucial importance. Such pairs could serve as specific probes for the screening of smaller molecules to identify drug candidates that may block the effect of the anthrax toxin. Development of an antigenic structure-based biochip of large capacity and diversity would facilitate efforts to identify such key structural elements and screen for their specific antibodies for drug development.

Thus, for the purpose of diagnosis, vaccination and drug development, it is important to recognize the dominant and minor antigenic determinants for a specific anti-infection antibody response to characterize the specificity and cross-reactivity of an antigenic structure; and to have a full-panel scanning of the repertoires of antibody specificities elicited by an infection. Such investigation has been impossible owing to a lack of a high throughput, multi-parameter assay system. Developing a carbohydrate and protein-based biochip to present a large repertoire of antigenic structures, including those displaying a single antigenic determinant on each microspot, would substantially facilitate these investigations.

(C) Antibody-Based Biochip

In principle, an antigen-based biochip is designed to detect and quantify specific antibodies and therefore, diagnose an infection. This method is not for the detection and quantification of antigens that are released from an infectious agent. Detecting microbial antigens in serum or other body fluids is generally more difficult than detecting antibodies. The former has, however, higher diagnostic value than the latter. It is, therefore, necessary to establish a highly sensitive antibody-based microarray to detect microbial antigens.

The complexity of a host anti-infection response further challenges the development of a multi-parameter immunoassay for characterizing infectious diseases. During an infection, a large panel of antigenic substances of distinct structural characteristic, including protein, polysaccharide, glycolipid, glycoproteins and nucleic acid, may be released to trigger the host immune response. These substances may differ significantly in their immunological properties and, therefore, elicit characteristic patterns of host responses. For example, protein antigens fail to elicit antibody responses in mice lacking a thymus but polysaccharides and other macromolecules with repetitive antigenic determinants can induce unimpaired antibody responses in these mice (30-32). The former are termed T-dependent (TD) antigens and the latter are T-independent (TI) antigens. The differences of TI- and TD-antigenic responses, which are recognizable by in vitro immunoassay, include a series of humoral factors, such as antigen specific antibodies, their Ig-isotypes, cytokines and other inflammation factors.

To better understand infectious disease, the full spectrum of anti-infection responses must be studied. These include specific antibody responses as well as the antigen-non-specific cytokine responses and other host responses. Cytokines are soluble proteins or glycoproteins, which play a critical role in controlling development or differentiation of lymphocytes and in regulating their anti-infection responses. For example, a microbial infection or vaccination may activate certain sub-types of T cells, either T helper 1 (Th1) cells or T helper 2 (Th2) cells. These specialized Th cells can produce unique profiles of cytotokines. Th1 secrete IL-2, IL-3, TNF-α and IFN-γ; Th2 secrete IL-3, IL-4, IL-5, IL-6, IL-9, IL-10 and some TNF-α. For anti-infection responses, induction of IgA-antibodies at mucosal sites is of critical importance. T cells and their cytokines have been shown to play a critical role in various stages of IgA response (33-35), including induction of Ig class switching of IgM to IgA and of terminal differentiation of IgA-committed B cells. Many researchers believe that IgA responses are highly Th-2 dependent (36-39), since there is evidence that Th2 cytokines, such as IL-4, IL-5 and IL-6, are required to induce terminal differentiation of IgA-committed B cells. By contrast, the Th1 cytokine, IFN-γ, antagonizes IL-4 in its IgA induction.

Our current efforts focus on the development of high throughput post-genomic technologies to extend the scope of biomedical research on human infectious diseases and the human immune response. A carbohydrate and protein-based microarray has been prepared, making it possible to display a large collection of antigens on a single biochip for probing the repertoires of antibody specificities and for studying carbohydrate and protein mediated molecular recognition on a large scale. This microarray platform has achieved the sensitivity to detect a broad range of human antibodies with as little as a few microliters of serum specimens and has reached the capacity to include antigenic preparations of most common pathogens. This technology is, therefore, readily applicable for large-scale production of an antigen/antibody microarray. In this invention, we describe the establishment of a detailed procedure for the industrial scale production of diagnostic biochips to enable simultaneous detection and characterization of a wide range of microbial infections, which include all listed Category A biological warfare agents. These include (a) the design and production of a carbohydrate-based microarray composed of microbial polysaccharides, its derivatives and a large panel of carbohydrate-containing macromolecules of distinct sugar structure; (b) a procedure to take advantage of the genomic information available for specific pathogens to design and produce proteomic microarrays to identify novel molecular targets for vaccination, diagnosis and drug development; (c) a method to produce an antibody-based microarray system to monitor microbial antigens in vivo and in vitro and to enable full-panel scanning of cytokines and other inflammation factors with limited amount of clinical specimens; and (d) the design and production of highly sensitive diagnostic biochips, including Diagnostic Biochip A and B for the rapid detection of all the biological warfare agents listed by the CDC as Category A pathogens and Diagnostic Biochip C for simultaneous detection and characterization of a wide range of common infectious diseases, which are caused by about 200 human pathogens.

II. Methods to Produce Large Capacity and High-Density Biochips (˜30,000 Microspots/Microchip)

Compared with DNA- and protein-based microarrays, the carbohydrate microarray technology has certain technical advantages and disadvantages. An obvious advantage is that the purified polysaccharides are generally stable in various conditions, either as dried solid or in aqueous solution, at room temperature or at 4° C. in storage. Unlike protein microarrays, wherein protein-denaturing and/or conformational alteration in microarray printing and storage is a major challenge to the technology, there is no such serious concern for most (if not all) carbohydrate antigens. A disadvantage of the carbohydrate microarray technology is that methods for high throughput production of carbohydrate macromolecules or complex carbohydrates, which is equivalent to the PCR for amplifying DNA or the cloning and expression method for producing proteins, are yet to be developed. Classical methods to obtain pure carbohydrate antigens include (a) isolation and purification from biological materials, such as cells, tissues or biological fluids; (b) chemical synthesis; and (c) enzymatic in vitro synthesis. A Rapid progress in establishing high throughput technology for the in vitro synthesis of carbohydrate macromolecules or complex carbohydrate molecules is most likely unexpected. The availability of purified carbohydrate antigens is, therefore, a potential rate-limiting factor to the carbohydrate microarray industry.

Our optimized methods for printing microarrays allows one: (i) to reduce amount of antigen needed for microarray printing; (ii) to increase the detection sensitivity and biochip capacity; (iii) to reduce the time that is necessary for a microarray printing cycle; (iv) to prevent cross-contaminations in printing microarrays; and (v) to reduce the cost of producing a diagnostic biochip substantially.

(A) Printing Microspots of Smaller Sizes and Hiqh-Densities

The general printing procedure is schematically illustrated in FIG. 9. A high-precision robot designed to produce cDNA microarrays was utilized to spot carbohydrate antigens onto glass slides that were pre-coated with nitrocellulose polymer. In addition, the following were also used:

a) STEALTH 3 pins for printing microspots 150 microns in diameter on nitrocellulose slides. This allows the arraying of 10,368 spots per FAST-slide with spot intervals of 250 micron, center-to-center; and

b) STEALTH 2.5 pins for printing spots at about 100 microns in diameter on the surface. 28,800 spots can be patterned on a single FAST slide. If each antigen preparation is printed as an array of four identical microspots, (See FIG. 12 below), 7,200 antigenic molecules can be included on a single slide. This system has, therefore, enough capacity to include most known human microbialpathogens and tumor-associated antigens.

III. Diagnostic Biochips

(A) Diagnostic Biochip A

This biochip is designed to enable the simultaneous detection of all Category A infectious diseases. In its clinical application, two-step staining is required. A number of carefully selected pairs of antigen and antibody are be printed on slides for each pathogen. The immobilized antigen serves as a probe to detect antibody in solution; the surface displayed antibody is used to capture a specific antigen in the solvent. This assay is, therefore, capable of detection of both antigens and antibodies in a single assay. Since an immobilized antigen may display multiple antigenic determinants, a microspot of antigen may capture antibodies that recognize different antigenic determinants expressed by the antigenic molecule. This makes the biochip system highly sensitive. Its detection specificity is, however, at the level of the antigenic molecule but not at the level of a single antigenic determinant. In case an antigen expresses a cross-reactive antigenic determinant, its detection specificity will be reduced (See the design of diagnostic biochip B for further discussion).

(1) 8-chamber sub-arrays (See FIG. 10):

Each micro-glass slide contains eight well-separated sub-arrays of identical contents. There are 600 microspots per sub-array, with spot sizes of approximately 200 micron at 300 micron intervals, center-to-center. A single slide is, therefore, designed to enable eight detections;

(2) Contains:

(a) Microspots: Each 600-spot sub-array is composed of carbohydrate and protein antigens, as well as antibodies specific to microbial antigens. The immobilized antigens will allow for the detection of human antibodies elicited by an infection; whereas immobilized antibodies are for the detection of microbial antigens;

(b) Repeats and dilutions: Each antigen/antibody will be printed at 0.5-1.0 mg/ml and at one to ten dilution of the initial concentration for the second concentration. Each preparation at a given concentration will be repeated three times; and

(c) Antibody isotype standard curves: Human antibodies of IgG, IgA and IgM isotype of known concentrations will serve as standard curves for antibody detection and normalization.

(3) Assay mechanisms:

(a) Detection of antibodies: use of immobilized antigens to capture antibodies in solution, which are then recognized by the tagged anti-human antibodies. In principle, this is an indirect immunoassay;

(b) Detection of microbial antigens: use of immobilized antibodies to capture antigens in solution and application of the tagged antibodies specific for the corresponding antigens to identify the captured antigens. This is known as a “Sandwich” immunoassay.

(4) Application and staining procedure:

Use of 0.5-1.0 microliters of serum specimens for the detection of a) specific antibodies in body fluids and b) presence of antigens in vivo and in vitro. Two steps of staining and approximately 5 hrs is required to complete the biochip analysis.

(5) Specificity & sensitivity:

a highly sensitive biochip system with specificity at the level of an antigenic molecule.

(B) Diagnostic Biochip B

This biochip is designed to enable the rapid diagnosis of the Category A infectious diseases, which requires only a single step of staining in clinical diagnosis. The time required for a biochip assay is, therefore, substantially shorter. Competitive immunoassay is used for rapid diagnosis. A competitive immunoassay requires an antigen/antibody pair. Either antigen or antibody can be immobilized on the solid surface, which will then interact with an antigen or antibody in solution. The immobilized antigen andtagged antibody in solution forms a specific probe to detect both antigen and antibody in clinical specimens. The free-antigen or antibody competitively inhibits the binding of the tagged antibody to the antigen immobilized on the solid surface. Key characteristics of this “competitive” one-step biochip that differ from Diagnostic Biochip A are summarized below:

(1y) Chip contents:

A panel of carefully selected microbial antigens, whose specific antibodies are available, will be printed on the chip with the method described above.

(2) Assay mechanisms:

The free antigens or antibodies can compete with the labeled antigen or antibody to bind the immobilized antigens or antibodies. Given that antibodies are generally more stable than other protein molecules in solution and are suitable for standardized production, printing antigen on a series of glass slides and using fluorescent-tagged antibodies for staining is preferred. If a clinical specimen in question contains a specific antibody for the target antigenic determinant or the antigen, the binding of the tagged antibody to the antigen will be competitively inhibited.

(3) Application and staining procedure:

One-step staining of the biochip allows detection of specific antibodies in body fluids of antigens in vivo and in vitro. The time required for biochip analysis, is therefore, reduced by 4 hours.

(4) Specificity & sensitivity:

Diagnostic Biochip B is a highly sensitive and specific biochip system. Its specificity is at the level of a single antigenic determinant.

C) Diagnostic Biochip C

Diagnostic Biochip C is composed of a large repertoire of carbohydrate and protein antigens as well as antibodies. This is a largely extended Diagnostic Biochip A. This biochip makes it possible to diagnose and characterize a wide range of microbial infections using a few microliters of serum specimen. This microarray has the printing capacity to include most common pathogens. Practically, it is limited by the availability of specific antigen preparations and their antibodies. 1000-2000 distinct antigens and antibodies can be printed on the chip. This enables a simultaneous detection of about 300 microbes, which include about 50-100 human pathogens. General properties of the master microarray are summarized as follows:

(1) Capacity:

15,000-20,000 microspots per micro-glass slide, with spot sizes approximately 150 micron in diameter and at 200-micron intervals, center-to-center.

(2) Diversity:

1000-1500 distinct antigenic preparations, about 100 antibodies specific for microbial antigens and about 30-50 antibodies for detecting human cytokines and other inflammation factors.

(3) Repeats and dilutions:

Each antigen has four dilutions (0.5 mg/ml to begin with) and each dilution has three repeats.

(4) Antibody standard curves:

Human antibodies of IgG, IgA and IgM isotype of known concentrations are printed on the chip to produce standard curves for normalization across the experiments and for quantitative calculation of the titer of the specific antibodies of a given isotype captured by the carbohydrate antigen.

(D) Diagnostic Biochip D

This biochip is composed of about 4,000 microspots of antigen and antibody preparations. Each antigen or antibody preparation of a given dilution was printed with four repeats in a vertical line of microspots on the biochip. This allows us to visually observe and statistically analyze the reproducibility of microarray printing and staining. The significance and sensitivity of antibody detection for each antigen at a given antigen concentration can also be statistically calculated. Some preparations, for example the gp120 glycoprotein of HIV-1 as highlighted in a square in FIG. 11, were printed from left to right in a series dilution of one to five, beginning at 0.5-1.0 mg/ml and with four dilutions thereafter. These biochips were stained with the normal or HIV-1 infected human serum specimens with the methods described above. Pictures of the ScanArray visualization of the multi-color fluorescent staining of biochips are shown in FIG. 11.

In this experiment, we have demonstrated that: (a) a procedure for microarray printing, antigen/antibody immobilization and antibody staining is precisely reproducible. On the same biochip, the correlation factors of data crossing different microspots of the same preparation is in the range of (0.98) to (1.00); (b) detection of human serum antibodies by this method is highly sensitive. A few microliters of serum specimen allows a full-panel scanning of the repertoires of human antibodies of different Ig-isotypes in a single assay. A large repertoire of antibody specificities were recognized in the normal and HIV infected individuals; (c) the specificity of this system is illustrated by recognizing the epitope-binding specificities of monoclonal anti-dextran antibodies and by specific detection of human serum antibodies for the gp120 glycoproteins and gag p24 of HIV-1 in AIDS patients; (d) a large repertoire of microbial antigens can be patterned on a single micro-glass slide, reaching the capacity to include most common and conditional pathogens; and (e) a biochip-based high throughput technology requires a strong bioinformatic presence to support it. While one may need a day to perform a biochip assay for a given clinical specimen, one may need many days to process the large amount of data produced by the biochip analysis. Developing advanced computer algorithms to facilitate this process is of equal importance with improving the hardware of the biochip technology.

(E) Diagnostic Biochip E

This is a proteomic microarray-based biochip produced with the microarray platform used to produce carbohydrate microarrays. Discovery of a large repertoire of genes in the genome of microorganisms has provided novel targets for vaccination, diagnosis, and drug development against microbial infection. To take advantage of this, HIV-1, whose genome is relatively small and has been completely sequenced, is used herein to establish the protein-based microarray technology. A large panel of purified HIV proteins, including gp120 proteins derived from different clades of HIV-1, Gag p55, p24, P6, P7, P17 and/or their E. coli expressed GST-fusion proteins, Tat, Nef, Integrase, and reverse transcriptase (RT), were printed and immobilized on the chemically modified glass slides. These protein chips were then applied to probe antibodies in normal and AIDS patients. As shown in FIG. 12, most HIV proteins printed on the chi gave positive detection of antibodies in HIV-infected individuals but not in normal controls, showing the sensitivity and specificity of this protein chip.

Therefore, we conclude that: (a) these positively stained HIV proteins were stably immobilized on the biochip and retained their immunological properties; (b) the protein microarray system has reached the sensitivity to probe the repertoires of antibodies in clinical specimens; and, most importantly, (c) it is feasible to introduce a large panel of protein products of genes, including both surface and non-surface proteins, and newly discovered genes, for our biochip production and diagnostic application. This finding is significantly important since it allows us to explore the application of genomic information and genetic material that has been accumulated by the genome projects.

(F) Diagnostic Biochip F

This is an antibody-based microarray and is designed to enable a full-panel scanning of human cytokine. Cytokine detection at the protein level is technically difficult. These proteins are potent biological agents that function at very low concentrations (7). Some cytokine molecules are unstable after secretion or activation (7). A highly sensitive specific assay is required to detect these molecules. A method currently used for cytokine detection, either ELISA-based assays or radioimmunoassays, takes advantage of the Sandwich-antibody assay. Specifically, the first cytokine-specific antibody is immobilized on a solid surface to capture cytokine in solution and then the second anti-cytokine antibody is applied to detect the surface-immobilized cytokine. The second anti-cytokine can be biotinylated, allowing signal amplification with a labeled Streptavidin. These methods are highly sensitive but limited in detecting a cytokine on a one-by-one base.

We have extended our microarray platform to produce the antibody-based biochips. The arrayed antigens allow specific capture of antibodies from body fluids and the antibodies immobilized on the glass chip can be used to detect soluble antigens and host factors, such as cytokines and other inflammatory factors. We immobilized a panel of anti-dextran mAbs at a concentration of 0.5 mg/ml on a set of glass slides. These include nitrocellulose-coated glass slides, polylysine treated slides, silane-treated glass slides and untreated, precleaned glass slides. These slides were then reacted with the fluorescence-tagged dextran preparations of distinct structures. Only the nitrocellulose-slides showed spots of specific fluorescent signals. Thus, anti-dextran mAbs were immobilized on the nitrocellulose-glass slide and retained their antigen binding specificities.

We further addressed whether the nitrocellulose-coated slide can be applied to immobilize antibodies on a chip for long-term storage. To investigate the condition for the preservation of antibody microarrays, we placed these microarrays under different conditions: (a) in an air-dried condition at room temperature; and (b) in a blocking solution, stored at 4° C. After six months, we stained these antibody microarrays with tagged antigens. We found that both group (a) and group (b) slides preserved antigen-capturing activities and specificities. Signals obtained by the two groups differ, however, significantly, with the latter much stronger than the former. These experiments have indicated the potential of the nitrocellulose-based non-chemical immobilization method for antibody microarray production.

An antibody microarray for a full-panel scanning of human cytokines was prepared. Polyclonal and monoclonal antibodies specific for human cytokines are currently available through various resources, providing a strong base for developing an antibody microarray to enable a highly sensitive, high throughput, full-panel cytokine scan.

We have found that the current microarray platform favors detection of IgG antibodies but not IgM antibodies in solution. The former is an immunoglobulin (Ig) monomer and the latter is an Ig pentamer. One theory for this result is that the pore size of FAST slides is too small to allow IgM antibody to enter and bind antigen immobilized in the deeper layer of the 3D nitrocellulose network structure (see ref. 40 for a 3D illustration of the nitrocellulose coating). This finding is of significance since it provides information for the development of an improved surface for producing a diagnostic biochip.

IV. Methods to Search and Identify Novel Carbohydrate Targets for Diagnosis and Vaccination

A large collection of carbohydrate containing macromolecules are printed on the nitrocellulose-coated slides and then the carbohydrate microarrays are stained with antibody preparations or lectins to probe the microspots that display the antigenic determinants in question. The monoclonal and polyclonal antibodies elicited by a microbial antigen or by a pathogen, as well as serum specimens of an infected individual are all useful reagents for these analyses. A positively stained microspot indicates the presence of a target molecule or antigenic determinant. Further characterization of this antigen preparation could lead to the identification of a suitable diagnostic molecule for a given infectious disease.

We have employed B. anthracis as a model pathogen to illustrate our research approach, from the identification of a target molecule to the further characterization of the structure using our carbohydrate microarray technology.

(A) B. Anthracis Related Carbohydrate Antigens as Diagnostic Molecular Targets

Carbohydrate structures present in B. anthracis are potential molecular targets for vaccine development and for diagnostic application. These carbohydrate structures include glycoproteins and polysaccharides. Glycoproteins are expressed by the dormant spore of B. anthracis and are recognizable by specific lectins, such as Glycine max (41). Since the spore surface interacts with the host initially in an anthrax infection, these structures are likely the targets for host recognition and antibody responses. They are therefore important for both vaccination and diagnosis. Polysaccharides are expressed by the germinating spores of B. anthracis, which are detected by monoclonal antibodies raised against the cell wall Gal-NAG polysaccharide of the pathogen (22). The exo- and/or cell wall Gal-NAG polysaccharide, which is present in the culture medium for growing B. anthracis (4), can be isolated from the cell wall of the microbe (22,42). Given that the Gal-NAG polysaccharide is universally present among and specific for strains of Bacilluus anthracis (22), its presence in solution and its structural stability, makes this molecule a potential target for the identification of B. anthracis and for developing an anthrax vaccine.

Early studies were conducted by the late Professors Michael Heidelberger (see ref. 43 page 451 for a description) and Elvin A. Kabat (44-46) to investigate the antigenic cross-reactivities among blood group substances (ABO), Pneumococcus type 14-polysaccharide and the cell wall polysaccharide of B. anthracis. A preparation of the anthrax polysaccharide is available in the KABAT collection at Columbia University. The anthrax polysaccharide was tested on a carbohydrate microarray and confirmed that the preparation has preserved its cross-reactivity to an anti-Pneumococcus type 14-polysaccharide antibody (data not shown).

Following are some important characteristics of the cell wall Gal-NAG polysaccharide: (a) it is 12,000 Da in molecular weight and contains galactose, N-acetylglucosamine, and N-acetylmannosamine in an approximate molar ratio of 3:2:1 (22) or 10:2:1. This slight difference in molar ratio is attributed to differences in the hydrolysis conditions applied (47); (b) it is pyruvylated although the sugar residue and position of pyruvylation are yet to be determined(47); (c) it is most likely B. anthracis specific and expressed by both germinating spores and the vegetative bacillus (22); (d) it shows antigenic determinants which are cross-reactive to Pneumococcus type 14 polysaccharide (43); (e) it has no human blood group substances A, B, or H activity (43); and (f) anti-serum elicited by the anthrax polysaccharide has, however, cross-reactivities with a preparation of hydrolyzed blood group substance A that contains the type II Gal-GlcNAc sequences (Gal β1→4G1cNAc) (see ref. 43, page 452 for a description of the study by Ivanovicx (1940)).

These characteristics lead to the conclusion that: (i) The Gal-NAG polysaccharide expresses potent antigenic determinants to mice and humans since the pyruvylated Gal-NAG structure is not present in the host; (ii) the polysaccharide itself is, however, poorly antigenic in solution since it is a T-independent antigen with relatively low molecular weight (1.2 KDa). Given this consideration, early observations that the polysaccharide was not protective to animals challenged by B. anthracis can be attributed to the nature of poor immunogenicity in its native configuration; (iii) its Gal-NAG core structure is similar to a core structure of blood group type II chain, Gal β1→GlcNAc. Pyruvylation of the backbone structure blocks, however, its blood group substance H reactivity; and (iv) given that the pyruvate group is not seen in Pneumococcus type 14 polysaccharide, the anthrax polysaccharide may contain a non-pyruvylated antigenic determinant mimicking those of the Pneumococcus type 14 polysaccharide. This leads us to conclude, therefore, that the cell wall polysaccharide of B. anthracis contains more than one antigenic determinant.

A carbohydrate microarray was designed to maximize the surface display of its potential antigenic structures using the following method: (a) print the polysaccharide on a microchip to display its antigenic determinants in the native configuration; (b) isolate the pyruvylated and non-pyruvylated fractions of the polysaccharide and apply them on the microarray as described by Mesnage et al. (47). This allows investigation of the potential dominant role of the pyruvylated sugar structure in its antigenicity; and (c) synthesize a panel of oligosaccharide-protein conjugates.

In the chip design, we have also included structures of oligosaccharides that are identical to or derived from those of Pneumococcus type 14 polysaccharide and those reactive to lectin Glycine max (specific for alpha-D-galactose or 2-acetamido-2-deoxy-alpha-D-galactose residues) that recognize a proposed spore glycoprotein of Bacilluus anthracis (41).

V. Methods to Search and Identify Pairs of Antigens and Antibodies for Antigen/antibody Microarrays

Detecting microbial antigens in serum or other body fluids is useful in the diagnosis of an infectious disease but generally it is difficult. It is, therefore, necessary to establish a highly sensitive antibody-based microarray to detect microbial antigens. In addition, such an antibody-based microarray will allow us to monitor a microbial pathogen in the environment as well as in host body fluids. Identification of highly specific antigens and antibodies for each pathogen is critical for the development of a diagnostic biochip.

We have established a simple and effective strategy to screen for these reagents for microarray production. We printed a panel of forty-eight carbohydrate-containing macromolecules of distinct structural characteristics on a slide with the method established above. In previous investigations, these antigens were successfully applied to screen human myeloma and lymphoma proteins for antibodies with anti-carbohydrate activities (48, 52, 53). The carbohydrate microarray was applied to detect human serum antibodies. A total number of twenty serum specimens were randomly collected from normal individuals. As little as one micro-liter of serum specimen from each individual was applied for microarray staining. As shown in FIG. 14, twelve distinct specificities of IgM antibodies (12/48) and thirty-five IgG anti-carbohydrate antibodies (35/48) were identified. Carbohydrate molecules that were positively stained include twenty polysaccharides (20/24), eleven complex carbohydrates of cellular origin (11/19) and four semi-synthetic glycoconjugates (4/5). For anti-carbohydrate antibodies of IgM isotype, twelve distinct specificities were identified. The majority (7/12) were bound to Klebsiella polysaccharides. This is similar to our previous observation that antibodies bound toKlebsiella polysaccharides were most frequently found in the repertoire of human myeloma anti-carbohydrate antibodies (48-52). The repertoire of human IgG anti-carbohydrate antibodies is, however, broader than those of IgM isotype. There are twenty specific for microbial polysaccharides (20/24), including polysaccharides of microbial pathogens, E. coli-K92 and -K100; Pneumococcus type- C, -VIII, -IX, -SIV, -XIV and -27; Group B Meningococcus, H. Influenza Type A, and different types of Klebsiella. Human IgG antibodies specific for glycomers of complex carbohydrate structures (11/19) and those for the carbohydrate moieties of semi-synthetic glycoconjugates (4/5) were also detected.

This antigen microarray was then applied to characterize monoclonal antibodies (FIG.14, panels III and IV) to critically evaluate their antigenic or epitope-binding specificities and cross-reactivities. Antigenic cross-reactivity between microbial polysaccharide α(1,6)dextran and a preparation of Chondroitin sulfate B polysaccharide that was derived from the intestinal mucosa of porcine was demonstrated. This has led to the recognition of a novel cross-reactive molecular marker of microbes and host cell (11). In addition, these experiments demonstrated that semi-synthetic glycoproteins are applicable for the construction of a carbohydrate microarray. This is of critical importance since it enables the application of rationally designed synthetic oligosaccharides for microarray construction and allows a critical examination of the specificity and cross-reactivity of carbohydrate-mediated molecular recognition.

The flexible 8-chamber biochip system, as described above, is sufficient for this type of initial screening of a panel of molecular targets. In this format, all the available antigen preparations of the eight Category-A pathogens can be printed at various dilutions in a single sub-array chamber. A specific antibody, for example, a mouse monoclonal antibody bound to the anthrax polysaccharide, can be applied on the sub-array biochip to react with all the antigens on the chip. It may also be applied in a series of dilutions on different sub-array chambers. This simple experiment allows us to critically evaluate (a) antibody binding specificity and cross-reactivity; (b) the affinity of antigen-antibody interaction; and (c) the quality of the printed antigen and method for immobilizing the antigen.

If an antigen/antibody pair is qualified by the 8-chamber biochip analysis, a further evaluation is conducted using a large-scale biochip, which displays both microbial antigens and those from human and other mammalian species. The highly specific pairs of antigens and antibodies are candidates for the diagnosis of corresponding microbial infections. Those who have cross-reactivities with human tissue antigens may be also useful for printing microarrays. As summarized above, identification of such cross-reactivities may lead to a better understanding of the biological relationship of microbes and their hosts, as well as the pathogenesis mechanisms of an infectious disease.

VI. Methods to Search and Identify Novel Protein Targets for Diagnosis and Vaccination

As described above, we have already established that our microarray platform is useful for printing protein microarrays and have demonstrated the principle and method to apply a large panel of protein antigens to extend our scope of investigation of microbial infections. This investigation has provided insights into how one could incorporate the genomic information and genetic material that has been accumulated by the genome sequencing projects into the development of diagnostic protein biochips. We report here the establishment of a strategy to efficiently utilize the information from the various pathogen genome projects to assist in the identification of novel molecular targets for diagnosis and vaccination. The pX01 plasmid of B. anthracis will serve as a model to illustrate our strategy.

(A) Methods to Search and Identify Candidates for the Protein Microarray

Different categories of proteins can be used to produce the diagnostic protein microarray. For example, (a) both membrane-bound and secretory proteins of a pathogen, which may trigger the initial immune response in an infection, making them ideal targets for early diagnosis and vaccination; (b) non-surface expressed proteins, which could be useful in identifying and characterizing the progressive stages of an infection; and (c) the candidate genes that are unique to a given pathogen, namely the species-specific or stain-specific molecular targets, thereby enabling highly specific detection on the final biochip.

Two categories of bioinformatic software, predictive and comparative, are currently available on-line. A two-step analysis can be used to identify candidate gene products to be included on the microarray. First, the structure, cellular localization, and function of newly discovered gones of the genome sequencing projects is predicted. Then a comparative analysis to predict the specificity and potential cross-reactivity of the candidate genes selected by the above structure-function predictive analysis is performed. A list of “predictive” software tools that will help in extrapolating a protein's function are available on-line. These include (a) TMHMM (http://www.cbs.dtu.dk/services/TMHMM-2.0/) for the prediction of transmembrane helices in proteins; (b) NetOGlyc 2.0 (http://www.cbs.dtu.dk/services/NetOGlyc/) for the prediction of mucin type GalNAc O-glycosylation sites in mammalian proteins; (c) PredictProtein (http://www.embl-heidelberg.de/predict protein/predictprotein.html) for the prediction of secondary structure, solvent accessibility, and transmembrane segments; and (d) 3D-PSSM Web Server V 2.6.0 (htt://www.bmm.icne t.uk/˜3dpssm/) for protein fold recognition.

With the predicted location and structure of the protein now known, either through published gene annotation done upon completion of a genome sequence, or through predictive bioinformatic software, it is equally important to determine whether the gene of interest is unique to the species through a comparative proteomic analysis. This is crucial if we hope to design a biochip that can specifically identify if the patient is suffering from B. anthracis. A gene that is similar in both B. anthracis and B. subtilis would be a poor choice for the biochip, because it would lead to inconclusive results when deployed in the field. Therefore, careful study should be done in order to identify if genes are highly unique to the species, which would in turn result in a biochip with few cross-reactive ambiguous results. One example of a potentially valuable bioinformatic tool to address this problem is called the COGnitor program. This program takes an amino acid sequence and extrapolates its function by comparing the unknown protein's sequence of amino acids to amino acid sequences from other genes of characterized function in a series of genomes (54). Genes that are highly conserved in evolution would be identifiable using the COGnitor program; those that are unique to a given organism could be non-recognizable by a COGnitor search. Proteins that are not recognized by the COGnitor program are probably high priority candidates for diagnostic application since the proteins they encode are most likely species-specific antigens. For the genes that are recognized and classified by the program, additional steps of investigation must be taken to define whether they are suitable targets for a diagnostic use.

(B) A Model to Illustrate our Comparative Genomic Strategy to Identify Unique Genes

The pX01 plasmid of Bacilluus anthracis has been fully sequenced, and 46 of the 142 identified genes have been functionally characterized in the literature (16). This provides us with a prime example of how a complete DNA sequence, with some gene annotation, would be approached using our comprehensive bioinformatic strategy.

First, we use predictive bioinformatic software to determine the location and structural characteristics of the proteins from their amino acid sequences. One example of this is in predicting if a protein has transmembrane regions, as can be determined by TMHMM version 2.0 (55). We have selected the protein Px01-54 as an example. Okinaka et al. have given a description of this protein as a “S-layer precursor/surface layer protein.” This protein was run through the transmembrane predicting software TMHMM version 2.0 (see FIG. 15).

It would seem that this protein contains both a transmembrane region, as well as a large extracellular component. One could hypothesize that, based on this result, the protein is on the surface of the bacteria, and is an ideal candidate to be printed on a microarray because it may play a role in initiating a primary immune response. Therefore, even if Okinaka et al did not characterize the gene, we could use this software to identify it as an ideal surface target that could induce a primary immune response.

With the completion of the predictive stage for function and location of a protein, the next stage of bioinformatic analysis is conducted, namely to determine if the gene of interest is unique to an organism. As described above, the COGnitor program (54) can be used to identify the species-specific genes/proteins for printing the diagnostic protein chips. To test this idea, we have run the amino acid sequences of all the 46 annotated genes of Px01 plasmid of B. anthracis through the COGnitor program. We present the results from this test in Table 2.

Of the 46 genes analyzed, 34 could be recognized and classified according to the COGnitor program. A large number of the COG-classified genes are of category L, namely DNA replication, recombination and repair. Indeed, it is likely that such proteins contain highly conserved domains across many species. At the other extreme, twelve genes were not recognized by the COGnitor program, and were thereby not able to be classified into the COG classifications. These unclassifiable proteins have been listed as “No Cog” in Table 2. This “No Cog” classification indicates that these proteins have no similarity to other proteins currently in the database. Since the COG database includes 74,059 classified protein sequences from the genomes of 43 species of both prokaryotic and eukaryotic organisms (but excludes the human genome), a “No Cog” result suggests that the protein in question is more likely to be species or strain-specific to the pathogen. At the very least, one can claim that it is highly unlikely for a “No Cog” protein to contain a known evolutionarily conserved ancient domain that exists across many species' genomes. Being able to select against genes that exhibit such conserved domains is a welcome tool, for this could help to lower the probability of selecting a protein that may yield ambiguous results on the final biochip due to cross reactivity. Interestingly, the genes of the most unique lethal nature, as well as a series of surface antigens, are not recognized by the COGnitor program because they match no other species' genes. One example of an ideal gene to select has been presented, namely the px01-54 protein of B. anthracis. This protein is expressed on the surface of the organism according to Okinaka et al. (16), spans a membrane according to the TMHMM program, and is not recognized by the COGnitor program, making it more likely to be unique to the species.

In conclusion, combining knowledge from the published literature and bioinformatic software on a gene product's function, location, and ‘species unique’ level will greatly enhance the use of the final biochip because it will allow clear and accurate clinical diagnosis of a patient's immune response to a wide panel of pathogenic agents.

### References

1. Blaustein, R. O., Koehler, T. M., Collier, R. J. & Finkelstein, A. Anthrax toxin: channel-forming activity of protective antigen in planar phospholipid bilayers. Proc Natl Acad Sci U S A 86, 2209-2213. (1989).

2. Thorne, C. B. Bacilluse anthracis. In Bacillus subtilis and Other Gram-positive Bacteria, ed. AL Sonenshein, J A Hoch, R Losick., 113-124 (1993).

3. Escuyer, V. & Collier, R. J. Anthrax protective antigen interacts with a specific receptor on the surface of CHO-K1 cells. Infect Immun 59, 3381-3386. (1991).

4. Strange, R. E. & Belton, F. C. Studies on a protective antigen produced in vitro from bacillus anthracis: Purification and chemisty of the antigen. Bri. J. Exp. Pathol. 35, 153-165 (1953).

5. Mourez, M. et al. Designing a polyvalent inhibitor of anthrax toxin. Nat Biotechnol 19, 958-961. (2001).

6. Brown, P. O. & Botstein, D. Exploring the new world of the genome with DNA microarrays. Nature. Genetics. 21, 33-37 (1999).

7. DeRisi, J. L., Iyer, V. R. & Brown, P. O. Exploring the metabolic and genetic control of gene expression on a genomic scale. Science. 278, 680-686 (1997).

8. Ramsay, G. DNA chips: state-of-the art. Nature. Biotechnology. 16, 40-44 (1998).

9. MacBeath, G. & Schreiber, S. L. Printing proteins as microarrays for high-throughput function determination. Science 299, 1760-1763 (2000).

10. Lueking, A. et al. Protein microarrays for gene expression and antibody screening. Analytical. Biochemistry. 270, 103-111 (1999).

11. Wang, D., Liu, S., Trummer, B. J., Deng, C. & Wang, A. Carbohydrate microarray leading to the recognition of cross-reactive molecular markers of microbes and host cells. Nature Biotechnology 20 (2002).

12. Ekins, R. P. Multi-analyte immunoassay. J Pharm Biomed Anal 7, 155-168 (1989).

13. Stoll, D. et al. Protein microarray technology. Front Biosci 7, C13-32. (2002).

14. Volchkov, V. E. Ebola virus, complete genome. Institute of Virology, Philipps-University (2000).

15. Shchelkunov, S. N., Totmenin,A. V. and Sandakhchiev, L. S. Analysis of the nucleotide sequence of 23.8 kbp from the left terminus of the genome of variola major virus strain India-1967. Virus Res. 40, 169-183 (1996).

16. Okinaka, R. T. et al. Sequence and organization of pX01, the large Bacilluus anthracis plasmid harboring the anthrax toxin genes. J Bacteriol 181, 6509-6515. (1999).

17. Wang, D. & Kabat, E. A. in Structure of Antigens. , Vol. Three. (ed. M. H. V. V. Regenmortal) 247-276 (CRC Press, Boca Raton New York London Tokyo; 1996).

18. Wyatt, R. et al. The antigenic structure of the HIVgp120 envelope glycoprotein. Nature 393, 705-711 (1998).

19. Robbins, J. B. & Schneerson, R. Polysaccharide-protein conjugates: a new generation of vaccines. J. Infct. Dis. 161, 821-832 (1990).

20. Schneerson, R., Barrera, O., Sutton, A. & Robbins, J. B. Preparation, characterization, and immunogenicity of Haemophilus influenzae type b polysaccharide-protein conjugates. J Exp Med 152,361-376. (1980).

21. Gladstone, G. P. & Walton, E. Effect of iron on the bactericidal proteins from rabbit polymorphonuclear leukocytes. Nature 227, 849-851. (1970).

22. Ezzell, J. W., Jr., Abshire, T. G., Little, S. F., Lidgerding, B. C. & Brown, C. Identification of Bacilluus anthracis by using monoclonal antibody to cell wall galactose-N-acetylglucosamine polysaccharide. J Clin Microbiol 28, 223-231. (1990).

23. Finne, J., Leinonen, M. & Makela, P. H. Antigenic similarities between brain components and bacteria causing meningitis. Implications for vaccine development and pathogenesis. Lancet. 2, 355-357 (1983).

24. Finne, J. & Makela, P. H. Cleavage of the polysialosyl units of brain glycoproteins by a bacteriophage endosialidase. Involvement of a long oligosaccharide segment in molecular interactions of polysialic acid. Journal. of Biological. Chemistry. 260, 1265-1270 (1985).

25. Mandrell, R. E. et al. Lipooligosaccharides (LOS) of some Haemophilus species mimic human glycosphingolipids, and some LOS are sialylated. Infection. &. Immunity. 60, 1322-1328 (1992).

26. Mandrell, R. E. Further antigenic similarities of Neisseria gonorrhoeae lipooligosaccharides and human glycosphingolipids. Infection. &. Immunity. 60, 3017-3020 (1992).

27. Feizi, T. & Loveless, R. W. Carbohydrate recognition by Mycoplasma pneumoniae and pathologic consequences. Am. J. Respir. Crit. Care. Med. 154, S133-136. (1996).

28. Burrel, C. J. Serological markers of hepatitis B. Clin Gastroenterol 9, 47-63. (1980).

29. Boxall, E. H., Peterson, S., Diment, J., Graha m, S. E. & Shirley, J. A. A novel assay for hepatitis B e markers. J Med Virol 52, 280-285. (1997).

30. Basten, A. & Howard, J. G. in Contemporary Topics in Immunobiology, Vol. 2. (ed. A. J. S. Davies) 265 (Plenum, New York; 1973).

31. Humphrey, J. H., Parrott, D. M. V. & East, J. Studies on globulin and antibody production in mice thymectomised at birth. Immunology 7, 419-439 (1964).

32. Weissman, I. L., Gutman, G. A., Friedberg, S. H. & Jerabek, L. Lymphoid tissue architecture. III. Germinal centers, T cells, and thymus-dependent vs thymus-independent antigens. Adv. Exp. Med. Biol. 66, 229-237 (1976).

33. Beagley, K. W., Black, C. A., Dunkley, M. L. & McGhee, J. R. in Cytokine Regulation of Humoral Immunity. (ed. C. M. Snapper) 391-408 (John Wiley & Sons Ltd, Chichester, New York, Brisbane,Toronto, Singapore; 1996).

34. McGhee, J. R., Mestecky, J., Elson, C. O. & Kiyono, H. Regulation of IgA synthesis and immune response by T cells and interleukins. [Review]. Journal of Clinical Immunology 9, 175-199 (1989).

35. Mestecky, J. The common mucosal immune system and current strategies for induction of immune responses in external secretions. [Review]. Journal of Clinical Immunology 7, 265-276 (1987).

36. Mongini, P. K. A., Stein, K. E. & Paul, W. E. T-cell regulation of IgG subclass antibody production in response to T-independent antigens. J. Exp. Med. 153, 1-12 (1981).

37. Kagnoff, M. F. & Murray, P. D. T dependent induction of an IgA and IgM anti-polysaccharide response. Advances in Experimental Medicine & Biology, 155-167 (1987).

38. Ivars, F., Nyberg, G., Holmberg, D. & Coutinho, A. Immune response to bacterial dextrans. II. T cell control of antibody isotypes. Journal of Experimental Medicine 158, 1498-1510 (1983).

39. Ehrhardt, R. O. et al. Differential activation requirements of isotype-switched B cells. Eur. J. Immunol. 26, 1926-1934 (1996).

40. Tonkinson, J. L. & Stillman, B. A. Nitrocellulose: a tried and true polymer finds utility as a post-genomic substrate. Front Biosci 7, C1-C12. (2002).

41. Cole, H. B., Ezzell, J. W., Jr., Keller, K. F. & Doyle, R. J. Differentiation of Bacilluus anthracis and other Bacillus species by lectins. J Clin Microbiol 19, 48-53. (1984).

42. Cohen, S. et al. Attenuated nontoxinogenic and nonencapsulated recombinant Bacilluus anthracis spore vaccines protect against anthrax. Infect Immun 68, 4549-4558. (2000).

43. Smith, H. & Zwartouw, H. T. The polysaccharide from Bacilluus anthracis grown in vivo. Biochem J 63, 447-452 (1956).

44. Kabat, E. A., Bezer, A. E., Baer, H. & Knaub, V. Immunochemical studies on blood groups. VII. Chemical changes associated with destruction of blood group activity and enhancement of the type XIV cross-reactivity by partial hydrolysis of hog and human blood group A, B, and O, substances. J. Exp. Med. 88, 43-57 (1948).

45. Kabat, E. A., Howe, C. & Schiffman, G. Immunnochemical studies on blood groups. J. Am. Chem. Soc. 80, 6656 (1958).

46. Kabat, E. A. Immunochemical studies on blood groups. J. Immunol. 82, 358-372 (1958).

47. Mesnage, S. et al. Bacterial SLH domain proteins are non-covale ntly anchored to the cell surface via a conserved mechanism involving wall polysaccharide pyruvylation. Embo J 19, 4473-4484. (2000).

48. Kabat, E. A. et al. Human monoclonal macroglobulins with specificity for Klebsiella K polysaccharides that contain 3,4-pyruvylated-D-galactose and 4,6-pyruvylated-D-galactose. J. Exp. Med. 152, 979-995 (1980).

49. Kabat, E. A. et al. The epitope associated with the binding of the capsular polysaccharide of the group B meningococcus and of Escherichia coli K1 to a human monoclonal macroglobulin, IgM NOV . J. Exp. Med. 168, 699-711 (1988).

50. Kabat, E. A., Liao, J., Sherman, W. H. & Osserman, E. F. Immunochemical characterization of the specificities of two human monoclonal IgM's reacting with chondroitin sulfates. Carbohydr. Res. 130, 289-297 (1984).

51. Kabat, E. A., Liao, J., Shyong, J. & Osserman, E. F. A monoclonal IgM lambda macroglobulin with specificity for lacto-N-tetraose in a patient with bronchogenic carcinoma. J. Immunol. 128, 540-544 (1982).

52. Kabat, E. A. et al. A human monoclonal macroglobulin with specificity for α(2- - - -8)-linked poly-N-acetyl neuraminic acid, the capsular polysaccharide of group B meningococci and Escherichia coli K1, which crossreacts with polynucleotides and with denatured DNA. J. Exp. Med. 164, 642-654 (1986).

53. Nickerson, K. G., Liao, J. & Kabat, E. A. A monoclonal IgM kappa from a blood group B individual with specificity for alpha-galactosyl epitopes on partially hydrolyzed blood group B substance. Carbohydr. Res. 243, 345-357 (1993).

54. Tatusov, R. L., Koonin, E. V. & Lipman, D. J. A genomic perspective on protein families. Science 278, 631-637. (1997).

55. Krogh, A., Larsson, B., von Heijne, G. & Sonnhammer, E. L. Predicting transmembrane protein topology with a hidden Markov model: application to complete genomes. J Mol Biol 305, 567-580. (2001).

### Fourth Series of Experiments

HydroGe™ slides are produced by Packard BioScience for printing protein microarrays. We investigated whether Hydrogel can serve as an alternative substrate for producing carbohydrate microarrays and for producing HIV diagnostic protein microarrays.

I. Materials And Methods

A. Procedures for Printing and Staining of the Hydrogel-Based Biochips

(1) Method for Printing on Hydrogel-Coated Slides

The method for printing carbohydrates and proteins on Hydrogel-coated slides is essentially identical to that for printing on nitrocellulose slides. The only difference is that the pre-treatment of Hydrogel follows the method described by the manufacture as follows: (a) place HydroGel slides in 400° C. incubator for 20 minutes; and (b) remove prior to printing and allow slides to cool to room temperature for 5 minutes.

2) Method for Post-Printing Treatment

The procedures for post-printing treatment were modified from the manufacture's instruction as follows: (a) incubate arrays overnight in a humidified 30° C. chamber; (b) rinse briefly (for 20 seconds) in PBST (PBS +0.05% Tween 20), followed by 3, 30 minute washes with PBST. Rinse for 20 seconds in PBS (pH 7.4); and (c) dry slides by centrifugation at 1600 RPM for 5 minutes in a table-top centrifuge.

(3) Method for Staining

The methods for staining Hydrogel biochips are identical to those for staining nitrocellulose biochips.

II. Results and Discussion

Applicants have shown the following surprising results:

1) Printing proteins and carbohydrate-containing macromolecules on hydrogel can be performed using Cartesian's PIXSYS 5500A Microarryer (CHIPMAKER 4), using a contact arraying procedure.

2) Protein preparations, including antibodies, BSA, Avidin and HIV-1 gp120, RT and gag proteins, can be quantitatively immobilized on the hydrogel, providing fine spots of about 150 microns in diameter and thus making it possible to produce a high density protein microarray.

3) Carbohydrate preparations, including polysaccharides, glycosaminoglycans, glycoproteins, semi-synthetic glycoconjugates and glycolipids, can be quantitatively immobilized on the hydrogel, providing fine spots of about 200 microns in diameter and thus making it possible to produce a high density carbohydrate microarray.

4) The immobilized protein and carbohydrate antigens analyzed have their immunological properties well-preserved at the time the slides were stained using specific antibodies.

5) Use of the Hydrogel-based protein and carbohydrate microarrays is advantageous due to their low fluorescent background. A disadvantage of the Hydrogel substrate is its absorption of relative lower amounts of materials on the biochip. For the same antigen preparations, the fluorescent signals detected on Hydrogel-slides are much lower than those detected on the nitrocellulose slides.

6) When the ratios of fluorescent intensity over background of given microspots are calculated, the results of nitrocellulose-chips and those of Hydrogel-chips are closely correlated in most cases (see Table 4 for a comparison among antigens of distinct structural characteristics).

7) The nitrocellulose-based biochip favors detection of IgG antibodies but not IgM antibodies in solution. The former is an immunoglobulin (Ig) monomer; and the latter is an Ig pentamer. Such bias is not seen in the Hydrogel-based biochids. This finding is of significance since it provides information to improve the nitrocellulose surface for producing a diagnostic biochip.

TABLE 1
Structural properties of dextrans and inulin preparations

| Plain dextrans | | | FITC conjugates | | | |
|---|---|---|---|---|---|---|
| N279 | LD7 | B1299S | Dex-20K | Dex-70K | Dex-2000K | Inulin |

| | | | | | | | |
|---|---|---|---|---|---|---|---|
| Structural characteristics | | | | | | | |
| Molecular weight (kDa) | ˜10,000 | 42 | ˜20,000 | 19.6 | 71.2 | 2,000 | 3.2 |
| Sugar residue | Glucose | Glucose | Glucose | Glucose | Glucose | Glucose | Fructose |
| Conformational | Linear | Linear | Heavily | Linear | Linear | Linear | Linear |
| characteristics | chain | chain | branched | chain | chain | chain | chain |
| | dominant | only | | dominant | dominant | dominant | dominant (?) |
| Molar ratio (FITC:sugar) | 0 | 0 | 0 | 0.01 | 0.005 | 0.008 | 0.007 |
| Proportions of linkages (%) ^{a} | | | | | | | |
| α(1,6) Terminal | 5 | 0 | 31 | 5 | 5 | 5 | 0 |
| nonreducing end group | | | | | | | |
| α(1,6) Backbone | 90 | 100 | 32 | 90 | 90 | 90 | 0 |
| α(1,3)α(1,6) Backbone | 0 | 0 | 1 | 0 | 0 | 0 | 0 |
| α(1,2)α(1,6) Backbone | 0 | 0 | 5 | 0 | 0 | 0 | 0 |
| α(1,3) Branches | 5 | 0 | 1 | 5 | 5 | 5 | 0 |
| α(1,2) Branches | 0 | 0 | 30 | 0 | 0 | 0 | 0 |
| β(2,1) Linkage | 0 | 0 | 0 | 0 | 0 | 0 | 100 (?) |

^{a} Values are estimated from methylation and periodate-oxidation analysis (see ref. 18 for a summary).

TABLE 2
Microarray detection and characterization of human and murine anti-carbohy drate antibodies^{a}

| Antigen enicro- [symbol] pots | | | | I. Human IgM | | | II. Human IgG | | |
|---|---|---|---|---|---|---|---|---|---|
| Antigen name | Class^{c} | ID | Location | Mean | SD | int/Bk [symbol] | Mean | SD | int/Bk |
| Klebsiella type 7 | 1 | 1 | A1 | 19293 | 1786 | 1.12 | 32140 | 3367 | 3.86 |
| Klebsiella type K11 | 1 | 2 | A2 | 19560 | 3349 | 1.13 | 15262 | 7630 | 1.52 |
| Klebsiella type K13 | 1 | 3 | A3 | 39103 | 4354 | 2.17 | 25597 | 719 | 3.20 |
| Klebsiella type K21 | 1 | 4 | A4 | 22847 | 2131 | 1.27 | 29255 | 890 | 3.63 |
| Dudmans Rhizabium TA1 | 1 | 5 | A5 | 31625 | 2768 | 1.77 | 16198 | 693 | 2.06 |
| Chondroitin SO4 “B” | 2 | 6 | A6 | 17009 | 633 | 0.96 | 10830 | 411 | 1.40 |
| Pneumococcus type C | 1 | 7 | C1 | 17014 | 1661 | 0.98 | 25187 | 3499 | 3.02 |
| Pneumococcus type VIII | 1 | 8 | C2 | 17194 | 1407 | 0.98 | 12075 | 3754 | 1.47 |
| PneumococcuS type XIV | 1 | 9 | C3 | 17012 | 1262 | 0.96 | 12292 | 4288 | 1.52 |
| Cow 21(Blood group B) | 3 | 10 | C4 | 19336 | 2180 | 1.08 | 9280 | 838 | 1.15 |
| Bacto-agar 20° C. Ext | 1 | 11 | C5 | 19792 | 2512 | 1.09 | 9682 | 1276 | 1.20 |
| Arabino Galactan (Larch) | 1 | 12 | C6 | 16216 | 453 | 0.90 | 8599 | 474 | 1.08 |
| IM3-BSA^{d} | 4 | 13 | El | 19743 | 1898 | 1.11 | 14322 | 1750 | 1.70 |
| IM3-KLH^{d} | 4 | 14 | E2 | 18077 | 1185 | 1.01 | 10641 | 930 | 1.27 |
| Le [symbol] (N-110% 2x) | 3 | 15 | E3 | 17046 | 728 | 0.95 | 8960 | 605 | 1.08 |
| Beach P1(Blood group B) | 3 | 16 | E4 | 17419 | 525 | 0.97 | 10022 | 423 | 1.21 |
| Tij II | 3 | 17 | E5 | 17946 | 464 | 0.99 | 9400 | 503 | 1.14 |
| (Blood group B & Le^{e})) | | | | | | | | | |
| Ogd | 3 | 18 | E6 | 20682 | 2620 | 1.13 | 8819 | 459 | 1.08 |
| ASOR^{d} | 3 | 19 | G1 | 16555 | 449 | 0.93 | 8933 | 320 | 1.06 |
| LNT-BSA^{d} | 4 | 20 | G2 | 19053 | 1266 | 1.05 | 9010 | 287 | 1.08 |
| Phosphornannan | 1 | 21 | G3 | 17571 | 785 | 0.97 | 16124 | 923 | 1.93 |
| Meningococcus group B | 1 | 22 | G4 | 16747 | 620 | 0.93 | 8839 | 403 | 1.06 |
| H. infl. Type A | 1 | 23 | G5 | 17804 | 656 | 0.98 | 11007 | 208 | 1.33 |
| E. coli K92 | 1 | 24 | G6 | 17353 | 770 | 0.95 | 8785 | 328 | 1.07 |
| Klebsiella type A3 | 1 | 25 | I1 | 27018 | 9910 | 1.40 | 13001 | 6947 | 1.94 |
| Klebsiella type K12 | 1 | 26 | I2 | 32322 | 16450 | 1.69 | 11539 | 5029 | 1.72 |
| Klebsiella type K14 | 1 | 27 | I3 | 23360 | 1283 | 1.22 | 54557 | 2045 | 7.80 |
| Klebsiella type K33 | 1 | 28 | I4 | 54607 | 2574 | 2.60 | 22890 | 1259 | 3.37 |
| Chondroitin SO4 “A” | 2 | 29 | I5 | 18093 | 1252 | 0.99 | 7646 | 730 | 1.16 |
| Chondroitin SO4 “C” | 2 | 30 | I6 | 17699 | 983 | 0.97 | 7547 | 607 | 1.14 |
| Pneumococcus type SIV | 1 | 31 | K1 | 19002 | 662 | 1.00 | 11485 | 819 | 1.68 |
| Pneumococcus type IX | 1 | 32 | K2 | 18932 | 1303 | 1.00 | 10600 | 3827 | 1.56 |
| Pneumococcus type 27 | 1 | 33 | K3 | 23308 | 6222 | 1.24 | 13455 | 7158 | 1.97 |
| Cow 26 (Blood group B) | 3 | 34 | K4 | 19184 | 1743 | 1.03 | 8020 | 885 | 1.19 |
| H [symbol] Pornatie Galactan | 1 | 35 | K5 | 17885 | 722 | 0.97 | 7298 | 550 | 1.11 |
| H [symbol] Nemoralis Galactan | 1 | 36 | K6 | 18135 | 715 | 0.98 | 7412 | 416 | 1.10 |
| IM6-BSA^{d} | 4 | 37 | M1 | 18897 | 184 | 1.01 | 9991 | 784 | 1.41 |
| IM6-KLH^{d} | 4 | 38 | M2 | 17761 | 289 | 0.96 | 7857 | 544 | 1.13 |
| Le [symbol] (N-1 IO⁻₄ NaOH) | 3 | 39 | M3 | 18430 | 926 | 1.00 | 7857 | 626 | 1.11 |
| Cyst 9 (Blood group A) | 3 | 40 | M4 | 18182 | 552 | 0.99 | 9081 | 733 | 1.30 |
| Dextran N-150-N (60K) | 1 | 41 | M5 | 17527 | 717 | 0.96 | 7908 | 671 | 1.17 |
| Hog (Blood Group H) | 3 | 42 | M6 | 17791 | 1015 | 0.98 | 7706 | 506 | 1.13 |
| AGOR^{d} | 3 | 43 | O1 | 18218 | 305 | 0.98 | 7698 | 485 | 1.09 |
| Inulin | 1 | 44 | O2 | 17655 | 361 | 0.95 | 7636 | 326 | 1.08 |
| Levan (B-512E) | 1 | 45 | O3 | 18003 | 318 | 0.97 | 11807 | 2116 | 1.68 |
| Meningococcus group Y | 1 | 46 | O4 | 17278 | 841 | 0.95 | 7749 | 433 | 1.09 |
| E. coli K100 | 1 | 47 | O5 | 17518 | 1219 | 0.97 | 7442 | 506 | 1.11 |
| E. coli L100 | 1 | 48 | O6 | 17852 | 807 | 0.99 | 15823 | 2152 | 2.33 |
| Background (n = 200) | | | | 18267 | 844 | | 7522 | 727 | |
| Total number of positives: | | | | | | 12 | | | 35 |
| Antigen enicro- [symbol] pots | | | | III. Anti-Dex 4.3F1 | | | IV. Anti-Dex 16.4.12E | | |
| Antigen name | Class^{c} | ID | Location | Mean | SD | int/BK | Mean | SD | int/BK |
| Klebsiella type 7 | 1 | 1 | A1 | 9074 | 215 | 1.01 | 11432 | 324 | 1.02 |
| Klebsiella type K11 | 1 | 2 | A2 | 9584 | 837 | 1.06 | 11432 | 262 | 1.03 |
| Klebsiella type K13 | 1 | 3 | A3 | 23003 | 3573 | 2.56 | 12256 | 648 | 1.10 |
| Klebsiella type K21 | 1 | 4 | A4 | 8817 | 203 | 0.98 | 12487 | 367 | 1.12 |
| Dudmans Rhizabium TA1 | 1 | 5 | A5 | 8438 | 448 | 0.93 | 10901 | 226 | 0.98 |
| Chondroitin SO4 “B” | 2 | 6 | A6 | 59264 | 822 | 6.38 | 18063 | 935 | 1.62 |
| Pneumococcus type C | 1 | 7 | C1 | 8813 | 373 | 0.98 | 11256 | 271 | 1.00 |
| Pneumococcus type VIII | 1 | 8 | C2 | 8862 | 362 | 0.99 | 11450 | 512 | 1.02 |
| PneumococcuS type XIV | 1 | 9 | C3 | 8879 | 330 | 0.99 | 11359 | 375 | 1.02 |
| Cow 21(Blood group B) | 3 | 10 | C4 | 9020 | 325 | 1.00 | 11309 | 450 | 1.02 |
| Bacto-agar 20° C. Ext | 1 | 11 | C5 | 9106 | 251 | 1.02 | 11269 | 527 | 1.02 |
| Arabino Galactan (Larch) | 1 | 12 | C6 | 8564 | 433 | 0.97 | 10909 | 357 | 0.99 |
| IM3-BSA^{d} | 4 | 13 | El | 10490 | 258 | 1.17 | 65535 | 0 | 5.34 |
| IM3-KLH^{d} | 4 | 14 | E2 | 8629 | 390 | 0.98 | 62128 | 5069 | 5.42 |
| Le [symbol] (N-110% 2x) | 3 | 15 | E3 | 8742 | 287 | 1.00 | 11026 | 501 | 0.98 |
| Beach P1(Blood group B) | 3 | 16 | E4 | 8638 | 307 | 0.96 | 10928 | 561 | 0.97 |
| Tij II | 3 | 17 | E5 | 8556 | 202 | 0.95 | 11006 | 457 | 0.98 |
| (Blood group B & Le^{e})) | | | | | | | | | |
| Ogd | 3 | 18 | E6 | 8912 | 427 | 1.00 | 11148 | 705 | 0.99 |
| ASOR^{d} | 3 | 19 | G1 | 8627 | 547 | 0.96 | 10923 | 566 | 1.00 |
| LNT-BSA^{d} | 4 | 20 | G2 | 8509 | 497 | 0.96 | 11000 | 316 | 0.99 |
| Phosphornannan | 1 | 21 | G3 | 8585 | 483 | 0.96 | 11224 | 394 | 1.01 |
| Meningococcus group B | 1 | 22 | G4 | 8633 | 285 | 0.96 | 11115 | 623 | 0.99 |
| H. infl. Type A | 1 | 23 | G5 | 8637 | 378 | 0.95 | 11205 | 499 | 1.01 |
| E. coli K92 | 1 | 24 | G6 | 8714 | 396 | 0.97 | 11248 | 190 | 1.01 |
| Klebsiella type A3 | 1 | 25 | I1 | 10278 | 1120 | 1.12 | 11496 | 136 | 1.01 |
| Klebsiella type K12 | 1 | 26 | I2 | 9303 | 293 | 1.01 | 11504 | 226 | 1.01 |
| Klebsiella type K14 | 1 | 27 | I3 | 9687 | 343 | 1.04 | 11752 | 405 | 1.03 |
| Klebsiella type K33 | 1 | 28 | I4 | 16135 | 3620 | 1.70 | 11286 | 424 | 1.00 |
| Chondroitin SO4 “A” | 2 | 29 | I5 | 9443 | 451 | 1.01 | 11322 | 215 | 1.00 |
| Chondroitin SO4 “C” | 2 | 30 | I6 | 11936 | 3057 | 1.35 | 11810 | 396 | 1.05 |
| Pneumococcus type SIV | 1 | 31 | K1 | 9305 | 351 | 1.03 | 11388 | 94 | 1.00 |
| Pneumococcus type IX | 1 | 32 | K2 | 9345 | 697 | 1.02 | 11407 | 227 | 1.01 |
| Pneumococcus type 27 | 1 | 33 | K3 | 9104 | 914 | 1.00 | 11394 | 252 | 1.00 |
| Cow 26 (Blood group B) | 3 | 34 | K4 | 9296 | 916 | 1.01 | 11309 | 205 | 1.00 |
| H [symbol] Pornatie Galactan | 1 | 35 | K5 | 9186 | 917 | 0.99 | 11369 | 268 | 1.01 |
| H [symbol] Nemoralis Galactan | 1 | 36 | K6 | 9179 | 773 | 0.98 | 11234 | 165 | 1.00 |
| IM6-BSA^{d} | 4 | 37 | M1 | 10610 | 423 | 1.13 | 62509 | 2863 | 5.43 |
| IM6-KLH^{d} | 4 | 38 | M2 | 9303 | 250 | 1.00 | 13534 | 460 | 1.19 |
| Le [symbol] (N-1 IO⁻₄ NaOH) | 3 | 39 | M3 | 9223 | 216 | 0.98 | 11293 | 240 | 1.00 |
| Cyst 9 (Blood group A) | 3 | 40 | M4 | 9129 | 333 | 0.98 | 11571 | 689 | 1.04 |
| Dextran N-150-N (60K) | 1 | 41 | M5 | 57385 | 1630 | 6.02 | 12653 | 197 | 1.13 |
| Hog (Blood Group H) | 3 | 42 | M6 | 9604 | 288 | 1.01 | 11526 | 145 | 1.04 |
| AGOR^{d} | 3 | 43 | O1 | 9228 | 384 | 0.99 | 11327 | 360 | 1.00 |
| Inulin | 1 | 44 | O2 | 9274 | 391 | 0.99 | 11264 | 511 | 1.00 |
| Levan (B-512E) | 1 | 45 | O3 | 9493 | 190 | 1.00 | 11423 | 521 | 1.01 |
| Meningococcus group Y | 1 | 46 | O4 | 9158 | 80 | 0.97 | 11454 | 687 | 1.01 |
| E. coli K100 | 1 | 47 | O5 | 9193 | 222 | 0.97 | 11236 | 364 | 1.00 |
| E. coli L100 | 1 | 48 | O6 | 9157 | 163 | 0.96 | 11178 | 270 | 1.00 |
| Background (n = 200) | | | | 9161 | 356 | | 11252 | 365 | |
| Total number of positives: | | | | | | 4 | | | 4 |
| ^{a}Data of tour microerrays were statistically analyzed and the positive results were highlighted in Bold italics. For human a [symbol] antibody staining, a positive score is given if the mean fluore [symbol] intensity value at a micro-spot is significantly higher than the mean background at the identically stained microarray with the same fluorescent cotar. For the staining using mono [symbol] onal antibodies. a positive [symbol] oore is | | | | | | | | | |
| # given. If the mean flu [symbol] cent intensity value of a micro-spot is at least 1.5 [symbol] ds higher than the mean background. | | | | | | | | | |

^{b}Cerbohydrate antigens were classified and indicated in table as the follows:
1 for Poly [symbol] ide; 2 Glycosaminoglycan; 3 for Glycoprotein; and 4 for Semi-synthetic Glycoconjugate.
^{c}int/BK: Ratio of mean fluorescence intensity to mean background.
^{d}AGOR: Agalacto-o [symbol] d;
ASOR: Asi [symbol] o- [symbol] d;
IM: isoma [symbol] e oligossocharide;
KLM, keyhole li [symbol] t h [symbol] in;
LNT: lacto-N- [symbol] e;
OG: Ogunsheye 10% 2X (blood ground I activity).

TABLE 3
COGnitor Analysis of Annotated Genes on the PX01 Plasmid of B. anthraces.

| | | | | | | Cog. Information [symbol] Independently | |
|---|---|---|---|---|---|---|---|
| Chart from [symbol] et al [symbol] of [symbol] Oct. 1999 [symbol] | | | | | | and Added to the Chart | |
| DRF | Start | [symbol] | Btrand | Size( [symbol] ) | Description | Cog Class | Cog Category |
| 137 | 173709 | 173894 | 1 | 61 | [symbol] | R | [symbol] |
| 139 | 174581 | 174871 | 2 | 68 | [symbol] | O | [symbol] |
| 138 | 174800 | 174483 | 2 | 97 | [symbol] | K | [symbol] |
| 108 | 131830 | 132238 | 2 | 99 | [symbol] | K | [symbol] |
| 121 | 153808 | 153837 | 2 | 110 | [symbol] | F | [symbol] |
| 120 | 183818 | 184238 | 1 | 190 | [symbol] | L | [symbol] |
| 116 | 149232 | 148884 | 2 | 150 | Unknown [symbol] | No Cog | No Cog |
| 87 | 101962 | 102444 | 2 | 160 | Unknwon [symbol] | OC | [symbol] |
| 120 | 152064 | 152536 | 1 | 190 | [symbol] | L | [symbol] |
| 115 | 142410 | 142901 | 2 | 193 | [symbol] | L | [symbol] |
| 111 | 138228 | 120843 | 2 | 201 | [symbol] | No Cog | No Cog |
| 127 | 162232 | 162878 | 2 | 214 | [symbol] | L | [symbol] |
| 141 | 175563 | 178307 | 2 | 214 | [symbol] | L | [symbol] |
| 65 | 99638 | 100319 | 1 | 227 | Hypothetical protein (244 aa). [symbol] | R | Producted metal-dependent membrance [symbol] |
| 130 | 165317 | 166030 | | 237 | Hypothetical protein (251 aa). [symbol] | No Cog | No Cog |
| 98 | 118307 | 117131 | 1 | 274 | Pulative [symbol] | L | Putative [symbol] |
| 91 | 109000 | 100612 | 1 | 200 | Hypothetical protein (193 aa). [symbol] | I | [symbol] |
| 94 | 112518 | 113403 | 1 | 196 | [symbol] | M | UOP- [symbol] |
| 10 | 25124 | 20071 | 1 | 315 | [symbol] | L | [symbol] |
| 103 | 123018 | 123971 | 1 | 317 | [symbol] | L | [symbol] |
| 39 | 48912 | 45889 | 2 | 325 | [symbol] | L | [symbol] |
| 112 | 138540 | 138523 | 2 | 327 | Spare germination response [symbol] | No Cog | No Cog |
| 114 | 141002 | 142051 | 2 | 368 | Spare germination response [symbol] | No cog | No Cog |
| 132 | 187948 | 188033 | 1 | 381 | [symbol] | B | [symbol] |
| 138 | 172205 | 17300 | 1 | 364 | Response [symbol] ; | | |
| | | | | | 126230 [symbol] (44%) | | |
| 93 | 111374 | 112474 | 1 | 368 | [symbol] | M | [symbol] |
| 88 | 100777 | 101822 | 2 | 381 | Hypothetical protein (402 aa). [symbol] | R | [symbol] |
| 54 | 87634 | 68818 | 1 | 383 | [symbol] | No Cog | No Cog |
| 45 | 57659 | 58066 | 2 | 435 | [symbol] | D | Cell [symbol] |
| 96 | 113430 | 114701 | 1 | 443 | Pulative NDP- [symbol] | M | [symbol] |
| 23 | 31621 | 33006 | 2 | 461 | Polential ooding region (509 aa); [symbol] | L | [symbol] |
| 81 | 95978 | 97353 | 1 | 461 | [symbol] | M | [symbol] |
| 118 | 150042 | 151459 | 1 | 459 | [symbol] | No Cog | No Cog |
| 59 | 74089 | 75601 | 1 | 477 | [symbol] | N | Type IV [symbol] |
| 35 | 42103 | 43639 | 1 | 476 | [symbol] | L | [symbol] |
| 38 | 43808 | 46262 | 2 | 484 | [symbol] | L | [symbol] |
| 112 | 133480 | 140058 | 2 | 402 | Spare germination response, [symbol] | No Cog | No Cog |
| 7 | 6944 | 8190 | 2 | 546 | [symbol] | L | [symbol] |
| 00 | 108772 | 103730 | 1 | 652 | Hypothetical protein ( [symbol] | L | [symbol] |
| 110 | 133161 | 135455 | 2 | 764 | [symbol] | No Cog | No Cog |
| 122 | 164224 | 160876 | 2 | 866 | [symbol] | O | [symbol] |
| 107 | 127442 | 129871 | 1 | 809 | [symbol] | No Cog | No Cog |
| 142 | 170738 | 179300 | 2 | 887 | [symbol] | L | [symbol] |
| 110 | 143180 | 148110 | 1 | 972 | [symbol] | No Cog | No Cog |
| 79 | 91443 | 95111 | 1 | 1,222 | Hypothetical protein (557 aa). [symbol] | N | [symbol] |
| 13 | 15040 | 19002 | 2 | 1,320 | [symbol] | No Cog | No Cog |

TABLE 4.i
(Exp1121401HIV-ratio)
Nitrocellulose and hydrogel as substrates for carbohydrate and
protein microarrays

| | Ratio of fluorescent Intensity | | | | | |
|---|---|---|---|---|---|---|
| | over background of microspots | | | | | |
| | FAST Slides | | | hydrogel | | |
| Human IgG specific for | Mean | SD | Sum | Mean | SD | Sum |
| 1_Polysaccharides | 5.35 | 8.28 | 6383.02 | 5.17 | 10.16 | 6163.61 |
| (2-150) n = 149 | | | | | | |
| 2_Glycosaminoglycn | 1.74 | 1.08 | 181.25 | 1.81 | 0.63 | 188.30 |
| (GAG) (152-164) n = 13 | | | | | | |
| 3_Glycoprotein | 2.27 | 3.36 | 1836.22 | 2.79 | 4.88 | 2254.38 |
| (186-266) n = 101 | | | | | | |
| 4_Semi-synthatic | | | | | | |
| glycoconjugates | 1.57 | 1.02 | 313.67 | 2.01 | 1.63 | 402.20 |
| (248-292) n = 45 | | | | | | |
| 5_Glycolipides | 1.45 | 0.34 | 139.52 | 1.68 | 0.19 | 161.18 |
| (249-305) n = 58 | | | | | | |
| 6_HIV-1 proteins | 4.32 | 8.40 | 6051.71 | 2.61 | 6.19 | 3659.95 |
| (307-481) n = 175 | | | | | | |
| 7_Other proteins | 3.57 | 5.95 | 2428.71 | 3.44 | 6.47 | 2340.21 |
| (483-567) n = 85 | | | | | | |
| 8_Ig | 4.27 | 8.11 | 2186.80 | 6.94 | 38.21 | 3554.90 |
| (569-632) n = 64 | | | | | | |
| BK_Empty space | 1.24 | 0.11 | 951.36 | 1.61 | 0.44 | 1237.61 |
| selected | | | | | | |
| (634-729) n = 96 | | | | | | |
| BK_Saline spots | 1.44 | 0.54 | 553.80 | 1.86 | 1.88 | 716.07 |
| (test for cross- | | | | | | |
| contamination) | | | | | | |
| (731-778) n = 48 | | | | | | |
| Total (n = 834) | 3.42 | 5.58 | 2633.25 | 3.37 | 9.89 | 2588.30 |

What is claimed is:

1. A microarray comprising a nitrocellulose or Hydrogel support having affixed to its surface at discrete loci a plurality of compounds, wherein (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

2. The microarray of claim 1, wherein the nitrocellulose or Hydrogel support is selected from the group consisting of a chip, a slide, a filter, and a plate.

3. A microarray comprising a plurality of nitrocellulose or Hydrogel or Hydrogel supports, each support having one or a plurality of compounds affixed to its surface at a single discrete locus or a plurality of compounds affixed to its surface at discrete loci, wherein (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

4. The microarray of claim 3, wherein the nitrocellulose or Hydrogel or Hydrogel support is selected from the group consisting of a chip, a slide, a filter, a plate, and a bead.

5. The microarray of claim 1 or 3, wherein the number of discrete loci is at least 100.

6. The microarray of claim 1 or 3, wherein the number of discrete loci is at least 1000.

7. The microarray of claim 1 or 3, wherein the number of discrete loci is at least 10,000.

8. The microarray of claim 1 or 3, wherein a glycomer is affixed at at least one locus.

9. The microarray of claim 1 or 3, wherein an insoluble protein is affixed at at least one locus.

10. The microarray of claim 1 or 3, wherein a lectin is affixed at at leastone locus.

11. The microarray of claim 1 or 3, wherein an antibody is affixed at at least one locus.

12. The microarray of claim 1 or 3, wherein at each locus is affixed only one compound.

13. The microarray of claim 1 or 3, wherein at at least one locus is affixed a plurality of compounds.

14. The microarray of claim 1 or 3, wherein the microarray has affixed to its surface two or more compounds selected from the group consisting of a glycomer, an insoluble protein, a lectin and an antibody.

15. The microarray of claim 1 or 3, wherein the microarray has further affixed to its surface a compound selected from the group consisting of a soluble protein, a nucleic acid and a small molecule.

16. An article comprising a nitrocellulose or Hydrogel support having dextran affixed to its surface at discrete loci.

17. The article of claim 16, wherein the dextran is α(1,6)dextran.

18. A microarray comprising the article of claim 16, wherein at least one compound is affixed to the dextran at each discrete locus, the composition of compounds at each discrete locus differing from the composition of compounds at at least one other discrete locus.

19. The microarray of claim 18, wherein the nitrocellulose or Hydrogel support is selected from the group consisting of a chip, a slide, a filter, and a plate.

20. An article comprising a plurality of nitrocellulose or Hydrogel supports, each support having dextran affixed to its surface at one or more discrete loci.

21. The article of claim 20, wherein the dextran is α(1,6)dextran.

22. A microarray comprising the article of claim 20, wherein at least one compound is affixed to the dextran at each discrete locus, the composition of compounds at each discrete locus differing from the composition of compounds at at least one other discrete locus.

23. The microarray of claim 22, wherein the nitrocellulose or Hydrogel support is selected from the group consisting of a chip, a slide, a filter, a plate, and a bead.

24. The microarray of claim 18 or 22, wherein the number of discrete loci is at least 100.

25. The microarray of claim 18 or 22, wherein the number of discrete loci is at least 1000.

26. The microarray of claim 18 or 22, wherein the number of discrete loci is at least 10,000.

27. The microarray of claim 18 or 22, wherein a glycomer is affixed to the dextran at at least one locus.

28. The microarray of claim 18 or 22, wherein an insoluble protein is affixed to the dextran at at least one locus.

29. The microarray of claim 18 or 22, wherein a lectin is affixed to the dextran at at least one locus.

30. The microarray of claim 18 or 22, wherein an antibody is affixed to the dextran at at least one locus.

31. The microarray of claim 18 or 22, wherein at each locus is affixed only one compound.

32. The microarray of claim 18 or 22, wherein at at least one locus is affixed a plurality of compounds.

33. The microarray of claim 18 or 22, wherein the microarray has affixed to the dextran two or more compounds selected from the group consisting of a glycomer, an insoluble protein, a lectin and an antibody.

34. The microarray of claim 18 or 22, wherein the microarray has affixed to its surface a compound selected from the group consisting of a soluble protein, a nucleic acid and a small molecule.

35. A method of detecting in a sample the presence of one or more agents which specifically bind to one or mote known glycomers, which method comprises:
(a) contacting the sample with the microarray of claim 1 or 3, wherein each known glycomer is affixed at at least one discrete locus and wherein the contacting is performed under conditions which would permit an agent, if present in the sample, to specifically bind to its corresponding glycomer in the microarray; and
(b) determining whether any known glycomer in the microarray has an agent specifically bound thereto,
thereby detecting the presence of the one or more agents in the sample.

36. The method of claim 35, wherein the agent is an antibody which correlates with an inflammatory disease.

37. The method of claim 35, wherein the agent is an antibody which correlates with an infection.

38. The method of claim 35, wherein the agent is an antibody which correlates with the presence of a tumor.

39. The method of claim 35, wherein the method comprises detecting the presence of a plurality of agents in the sample, each of which binds to a plurality of glycomers.

40. The method of claim 35, wherein the method comprises determining the amount of a plurality of agents in the sample, each of which binds to one glycomer.

41. A method of detecting in a sample the presence of one or more agents which specifically bind to one or more known insoluble proteins, which method comprises:
(a) contacting the sample with the microarray of claim 1 or 3, wherein each known insoluble protein is affixed at at least one discrete locus and wherein the contacting is performed under conditions which would permit an agent, if present in the sample, to specifically bind to its corresponding insoluble protein in the microarray; and
(b) determining whether any known insoluble protein in the microarray has an agent specifically bound thereto,
thereby detecting the presence of the one or more agents in the sample.

42. The method of claim 41, wherein the agent is an antibody which correlates with a disease.

43. The method of claim 41, wherein the agent is an antibody which correlates with an infection.

44. The method of claim 41, wherein the agent is an antibody which correlates with the presence of a tumor.

45. The method of claim 41, wherein the method comprises detecting the presence of a plurality of agents in the sample, each of which binds to a plurality of insoluble proteins.

46. The method of claim 41, wherein the method comprises determining the amount of a plurality of agents in the sample, each of which binds to one insoluble protein.

47. A method of detecting in a sample the presence of one or more agents which specifically bind to one or more known antibodies or lectins, which method comprises:
(a) contacting the sample with the microarray of claim 1 or 3, wherein each known antibody or lectin is affixed at at least one discrete locus and wherein the contacting is performed under conditions which would permit an agent, if present in the sample, to specifically bind to its corresponding antibody or lectin in the microarray; and
(b) determining whether any known antibody or lectin in the microarray has an agent specifically bound thereto,
thereby detecting the presence of the one or more agents in the sample.

48. The method of claim 47, wherein the agent is an antibody which correlates with a disease.

49. The method of claim 47, wherein the agent is an antibody which corresponds to an infection.

50. The method of claim 47, wherein the agent is an antibody which correlates with the presence of a tumor.

51. The method of claim 47, wherein the method comprises detecting the presence of a plurality of agents in the sample, each of which binds to a plurality of lectins or antibodies.

52. The method of claim 47, wherein the method comprises determining the amount of a plurality of agents in the sample, each of which binds to one lectin or antibody.

53. A method of determining the amount of one or more agents in a sample, each of which specifically binds to one or more known glycomers, which method comprises:
(a) contacting the sample with the microarray of claim 1 or 3, wherein each known glycomer is affixed at at least one discrete locus, and wherein the contacting is performed under conditions which would permit an agent, if present in the sample, to specifically bind to its corresponding glycomer in the microarray;
(b) for each known glycomer in the microarray, determining the amount of agent specifically bound thereto; and
(c) comparing the amounts so determined to a known standard,
thereby determining the amount of the one or more agents in the sample.

54. The method of claim 53, wherein the agent is an antibody which correlates with an inflammatory disease.

55. The method of claim 53, wherein the agent is an antibody which correlates with an infection.

56. The method of claim 53, wherein the agent is an antibody which correlates with the presence of a tumor.

57. The method of claim 53, wherein the method comprises determining the amount of a plurality of agents in the sample, each of which binds to a plurality of glycomers.

58. The method of claim 53, wherein the method comprises determining the amount of a plurality of agents in the sample, each of which binds to one glycomer.

59. A method of determining the amount of one or more agents in a sample, each of which specifically binds to one or more known insoluble proteins, which method comprises:
(a) contacting the sample with the microarray of claim 1 or 3, wherein each known insoluble protein is affixed at at least one discrete locus, and wherein the contacting is performed under conditions which would permit an agent, if present in the sample, to specifically bind to its corresponding insoluble protein in the microarray;
(b) for each known insoluble protein in the microarray, determining the amount of agent specifically bound thereto; and
(c) comparing the amounts so determined to a known standard,
thereby determining the amount of the one or more agents in the sample.

60. The method of claim 59, wherein the agent is an antibody which correlates with an inflammatory disease.

61. The method of claim 59, wherein the agent is an antibody which correlates with an infection.

62. The method of claim 59, wherein the agent is an antibody which correlates with the presence of a tumor.

63. The method of claim 59, wherein the method comprises determining the amount of a plurality of agents in the sample, each of which binds to a plurality of insoluble proteins.

64. The method of claim 59, wherein the method comprises determining the amount of a plurality of agents in the sample, each of which binds to one insoluble protein.

65. A method of determining the amount of one or more agents in a sample, each of which specifically binds to one or more known antibodies or lectins, which method comprises:
(a) contacting the sample with the microarray of claim 1 or 3, wherein each known antibody or lectin is affixed at at least one discrete locus, and wherein the contacting is performed under conditions which would permit an agent, if present in the sample, to specifically bind to its corresponding antibody or lectin in the microarray;
(b) for each known antibody or lectin in the microarray, determining the amount of agent specifically bound thereto; and
(c) comparing the amounts so determined to a known standard,
thereby determining the amount of the one or more agents in the sample.

66. The method of claim 65, wherein the agent is an antibody which correlates with an inflammatory disease.

67. The method of claim 65, wherein the agent is an antibody which correlates with an infection.

68. The method of claim 65, wherein the agent is an antibody which correlates with the presence of a tumor.

69. The method of claim 65, which method comprises determining the amount of a plurality of agents in the sample, each of which binds to a plurality of lectins or antibodies.

70. The method of claim 65, wherein the method comprises determining the amount of a plurality of agents in the sample, each of which binds to one lectin or antibody.

71. A method of determining whether a subject is afflicted with a disorder characterized by the presence or absence in an afflicted subject of an agent which specifically binds to a known glycomer, which method comprises:
(a) contacting a suitable sample from the subject with the microarray of claim 1 or 3, wherein the known glycomer is affixed at at least one discrete locus and wherein the contacting is performed under conditions which would permit the agent, if present in the sample, to specifically bind to the known glycomer in the microarray; and
(b) determining whether the known glycomer in the microarray has the agent specifically bound thereto,
thereby determining whether the subject is afflicted with the disorder.

72. The method of claim 71, wherein the subject is human.

73. The method of claim 71, wherein the disorder is an inflammatory disorder.

74. The method of claim 73, wherein the inflammatory disorder is celiac disease.

75. A method of determining whether a subject is afflicted with a disorder characterized by the presence or absence in an afflicted subject of an agent which specifically binds to a known insoluble protein, which method comprises:
(a) contacting a suitable sample from the subject with the microarray of claim 1 or 3, wherein the known insoluble protein is affixed at at least one discrete locus and wherein the contacting is performed under conditions which would permit the agent, if present in the sample, to specifically bind to the known insoluble protein in the microarray; and
(b) determining whether the known insoluble protein in the microarray has the agent specifically bound thereto,
thereby determining whether the subject is afflicted with the disorder.

76. The method of claim 75, wherein the subject is human.

77. A method of determining whether a subject is afflicted with a disorder characterized by the presence or absence in an afflicted subject of an agent which specifically binds to a known antibody or lectin, which method comprises:
(a) contacting a suitable sample from the subject with the microarray of claim 1 or 3, wherein the known antibody or lectin is affixed at at least one discrete locus and wherein the contacting is performed under conditions which would permit the agent, if present in the sample, to specifically bind to the known antibody or lectin in the microarray; and
(b) determining whether the known antibody or lectin in the microarray has the agent specifically bound thereto,
thereby determining whether the subject is afflicted with the disorder.

78. The method of claim 77, wherein the subject is human.

79. The method of claim 77, wherein the disorder is HIV-1 infection.

80. A method of determining whether an antibody known to specifically bind to a first glycomer also specifically binds to a second glycomer, which method comprises:
(a) contacting the antibody with the microarray of claim 1 or 3, wherein a plurality of glycomers, other than the first glycomer, are affixed at discrete loci in the microarray, and wherein the contacting is performed under conditions which would permit the antibody to specifically bind to the first glycomer if it were present in the microarray; and
(b) determining whether any of the glycomers in the microarray, other than the first glycomer, has the antibody specifically bound thereto,
thereby determining whether the antibody also specifically binds to a second glycomer.

81. A method of determining whether an antibody known to specifically bind to a first insoluble protein also specifically binds to a second insoluble protein, which method comprises:
(a) contacting the antibody with the microarray of claim 1 or 3, wherein a plurality of insoluble proteins, other than the first insoluble protein, are affixed at discrete loci in the microarray, and wherein the contacting is performed under conditions which would permit the antibody to specifically bind to the first insoluble protein if it were present in the microarray; and
(b) determining whether any of the insoluble proteins in the microarray, other than the first insoluble protein, has the antibody specifically bound thereto,
thereby determining whether the antibody also specifically binds to a second insoluble protein.

82. A method of making a microarray comprising a nitrocellulose or Hydrogel support having affixed to its surface at discrete loci a plurality of compounds, which method comprises contacting the nitrocellulose or Hydrogel support with the compounds under suitable conditions, whereby (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

83. A method of making a microarray comprising a plurality of nitrocellulose or Hydrogel supports, each support having one or a plurality of compounds affixed to its surface at a single discrete locus or a plurality of compounds affixed to its surface at discrete loci, which method comprises contacting the nitrocellulose or Hydrogel supports with the compounds under suitable conditions, whereby (a) at at least one discrete locus is affixed a compound selected from the group consisting of a glycomer, an insoluble protein, a lectin and an antibody, and (b) the composition of compounds at each discrete locus differs from the composition of compounds at at least one other discrete locus.

84. A method of making the article of claim 16 comprising contacting a nitrocellulose or Hydrogel support with dextran at discrete loci under suitable conditions.

85. The method of claim 84, further comprising the step of affixing at least one compound to the dextran at each discrete locus, whereby the composition of compounds at each discrete locus differs from the composition of compounds at at least one other discrete locus.

86. A method of making the article of claim 20 comprising contacting a plurality of nitrocellulose or Hydrogel supports with dextran, whereby each support has dextran affixed to its surface at one or more discrete loci.

87. The method of claim 86, further comprising the step of affixing at least one compound to the dextran at each discrete locus, whereby the composition of compounds at each discrete locus differs from the composition of compounds at at least one other discrete locus.

88. A kit comprising the microarray of claim 1, 3, 18 or 22 and instructions for use.

89. A kit comprising the microarray of claim 1, 3, 18 or 22 and a desiccant.

90. A kit comprising the microarray of claim 1, 3, 18 or 22 immersed in an aqueous solution.

91. A kit for practicing the method of claim 71, which comprises:
(a) a microarray comprising a nitrocellulose or Hydrogel support having affixed to its surface at discrete loci a plurality of compounds, wherein (i) at at least one discrete locus is affixed the glycomer to which the agent present or absent in an afflicted subject specifically binds, and (ii) the composition of compounds at each discrete locus differs from the composition of compounds at at least one other discrete locus; and
(b) instructions for use.

92. A kit for practicing the method of claim 75, which comprises:
(a) a microarray comprising a nitrocellulose or Hydrogel support having affixed to its surface at discrete loci a plurality of compounds, wherein (i) at at least one discrete locus is affixed the insoluble protein to which the agent present or absent in an afflicted subject specifically binds, and (ii) the composition of compounds at each discrete locus differs from the composition of compounds at at least one other discrete locus; and
(b) instructions for use.

93. A kit for practicing the method of claim 77, which comprises:
(a) a microarray comprising a nitrocellulose or Hydrogel support having affixed to its surface at discrete loci a plurality of compounds, wherein (i) at at least one discrete locus is affixed the antibody or lectin to which the agent present or absent in an afflicted subject specifically binds, and (ii) the composition of compounds at each discrete locus differs from the composition of compounds at at least one other discrete locus; and
(b) instructions for use.

94. An antibody capable of specifically binding to a glycomer present on the surface of a mammalian macrophage, which glycomer, or structural mimic thereof, is also endogenous to, and present on the surface of, a bacterial cell.

95. The antibody of claim 94, wherein the antibody is a groove-type antibody.

96. The antibody of claim 94, wherein the antibody is designated 4.3. F1 (ATCC Accession No. PTA-3259).

97. The antibody of claim 94, wherein the antibody is designated 45.21.1 (ATCC Accession No. PTA-3260).

98. An antibody capable of specifically binding to a glycomer present on the surface of a mammalian intestinal epithelial cell, which glycomer, or structural mimic thereof, is also endogenous to, and present on the surface of, a bacterial cell.

99. The antibody of claim 98, wherein the antibody is a cavity-type antibody.

100. The antibody of claim 98, wherein the antibody is designated 16.4.12E (ATCC Accession No. PTA-3261).

101. A method of determining whether a subject is afflicted with a disorder characterized by the presence of a glycomer on the surface of macrophages in an afflicted subject, which glycomer, or structural mimic thereof, is also endogenous to, and present on the surface of, a bacterial cell, comprising:
(a) contacting a sample of the subject's macrophages with the antibody of claim 94; and
(b) determining whether the antibody specifically binds to the macrophages in the sample,
such binding indicating that the subject is afflicted with the disorder.

102. The method of claim 101, wherein the subject is human.

103. The method of claim 101, wherein the disorder is an immune disorder or an inflammatory disorder.

104. A method of determining whether a subject is afflicted with a disorder characterized by the presence of a glycomer on the surface of intestinal epithelial cells in an afflicted subject, which glycomer, or structural mimic thereof, is also endogenous -to, and present on the surface of, a bacterial cell, comprising:
(a) contacting a sample of the subject's intestinal epithelial cells with the antibody of claim 98; and
(b) determining whether the antibody specifically binds to the intestinal epithelial cells in the sample,
such binding indicating that the subject is afflicted with the disorder.

105. The method of claim 104, wherein the subject is human.

106. The method of claim 104, wherein the disorder is an immune disorder or an inflammatory disorder.

107. The method of claim 106, wherein the disorder is celiac disease.
